(19) United States
(12) Patent Application Publication
(10) Pub. No.: US 2018/0296491 A1
(43) Pub. Date: Oct. 18, 2018

(54) NANOPARTICLE THERAPEUTICS FOR TREATING SKIN INFLAMMATORY DISORDERS

(71) Applicant: University of Rochester, Rochester, NY (US)

(72) Inventors: Lisa DeLouise, Rochester, NY (US); Samreen Jatana, Rochester, NY (US); Brian Palmer, Rochester, NY (US)

(21) Appl. No.: 15/569,468

(22) PCT Filed: Apr. 27, 2016
(86) PCT No.: PCT/US16/29567

§ 371 Date: Oct. 26, 2017

Related U.S. Application Data
(60) Provisional application No. 62/221,834, filed on Sep. 22, 2015.
(60) Provisional application No. 62/156,559, filed on May 4, 2015.
(60) Provisional application No. 62/153,279, filed on Apr. 27, 2015.

(51) Int. Cl. A61K 9/51; A61K 9/00; A61P 37/06; A61P 37/08; A61P 17/00; A61K 33/00

(52) U.S. Cl. CPC A61K 9/51; A61K 9/0014; A61K 33/00; A61P 37/08; A61P 17/00; A61P 37/06

(57) ABSTRACT

The present invention provides nanoparticles and methods for treating and preventing skin inflammatory conditions or disorders. The conditions or disorders include allergic contact dermatitis (ACD), irritant contact dermatitis, atopic dermatitis (AD), photoallergic dermatitis, and contact hypersensitivity (CHS), as well as other conditions or disorders associated with the skin.

### CROSS-REFERENCE TO RELATED APPLICATIONS

This application claims priority to U.S. Provisional Patent Application No. 62/153,279 filed Apr. 27, 2015, to U.S. Provisional Patent Application No. 62/156,559 filed May 4, 2015, and to U.S. Provisional Patent Application No. 62/221,834 filed Sep. 22, 2015, the contents of which are incorporated by reference herein in their entirety.

### STATEMENT REGARDING FEDERALLY SPONSORED RESEARCH OR DEVELOPMENT

This invention was made with government support under grant numbers 1RO1ES021492 and ES-07026 awarded by the National Institutes of Health. The government has certain rights in the invention.

### BACKGROUND OF THE INVENTION

Skin is the main route to allergen sensitization and provides innate as well as adaptive immune functions to maintain homeostasis. Skin antigen presenting cells (APCs) generate an immune response following allergen exposure as in the case of Allergic Contact Dermatitis (ACD). APCs sensitize effector CD4+ and CD8+ T cells in the lymph nodes against topical allergens at the point of first contact and a subsequent exposure can cause pruritic rash and/or swelling generated by the antigen-specific T cells.

Common environmental and work place contact allergens include urushiol in poison ivy, nickel in jewelry, various rubbers, resins, acrylics, and adhesives. Contact dermatitis is 1 of the 10 leading occupational illnesses. With the expanding use of engineered NPs (<100 nm) in consumer products and technological applications are increasing toxicity concerns associated with direct skin contact alone or in the presence of a chemical sensitizer. Examples of nano-enabled consumer products include ZnO and TiO₂ particles used to formulate sunscreens, nano-silver (Ag) to make antimicrobial textiles and wound care dressings and carbon nanotubes (CNT) to make high strength sporting equipment (bikes, racquets) (Delouise L A, J Invest Dermatol, 2012, 132:964-975).

All skin inflammatory disorders are routinely treated with steroids and/or potent calcineurin inhibitors. The goal of treatment is to reduce symptoms that include swelling, redness, barrier dysfunction, pruritus (itch), and induration (tissue hardening). Steroidal effects are nonspecific and often ineffective, especially in situations where the contact sensitizer is either unidentified or unavoidable as may be the case for skin contact with sensitizers that are hard to detect and control in the work place and in the environment. Serious adverse side-effects such as skin atrophy and skin barrier dysfunction result from prolonged topical steroid use. Potent calcineurin inhibitors suppress T cell activity but they must be administered under the close supervision of a doctor as patients may develop adverse side-effects from long term use and are at risk for developing cancer.

There is a need in the art for improved methods of treating skin disorders. The present invention meets this need.

### SUMMARY OF THE INVENTION

The present invention provides nanoparticles and methods for treating and preventing skin inflammatory conditions or disorders. The conditions or disorders include allergic contact dermatitis (ACD), irritant contact dermatitis, atopic dermatitis (AD), photoallergic dermatitis, and contact hypersensitivity (CHS), as well as other conditions or disorders associated with the skin.

In one aspect, the present invention relates to a method of treating skin inflammation in a subject in need thereof, the method comprising: administering a therapeutically effective amount of a composition comprising at least one nanoparticle (NP) to a site of skin inflammation of the subject.

In one embodiment, the at least one NP is selected from the group consisting of: silica nanosphere, porous silicon nanoshard, quantum dot, gold nanoparticle, and silver nanoparticle. In one embodiment, the at least one NP is the quantum dot comprising a neutrally charged coating, or a negatively charged coating, or a glutathione coating. In one embodiment, the at least one NP is a quantum dot that is lipophilic, organic, or a cadmium-selenide/zinc sulfide (CdSe/ZnS) quantum dot capped with octadecyl amine ligands (ODA). In one embodiment, the silica nanosphere has a diameter of about 10 nm to about 1200 nm. In one embodiment, the silica nanosphere has a diameter of about 20 nm to about 400 nm. In one embodiment, the porous silicon nanoshard has a porosity between about 20% and about 80%. In one embodiment, the porous silicon nanoshard has a diameter of about 1 nm to about 1000 nm. In one embodiment, the porous silicon nanoshard has a diameter of about 20 nm to about 400 nm. In one embodiment, the composition is administered topically. In one embodiment, the skin inflammation is associated with at least one selected from the group consisting of: chemical irritation, contact dermatitis, and an autoimmune disorder. In one embodiment, the skin inflammation is associated with at least one selected from the group consisting of allergic contact dermatitis (ACD), irritant contact dermatitis, atopic dermatitis (AD), photoallergic dermatitis, and contact hypersensitivity (CHS). In one embodiment, the skin inflammation comprises at least one of: swelling, redness, barrier dysfunction, pruritus (itch), and induration (tissue hardening).

In another aspect, the invention relates to a composition for the treatment of skin inflammation, the composition comprising an effective amount of at least one nanoparticle (NP), wherein the at least one NP suppresses an immune response in skin.

In one embodiment, the at least one NP is selected from the group consisting of: silica nanosphere, porous silicon nanoshard, quantum dot, gold nanoparticle, and silver nanoparticle. In one embodiment, the at least one NP is a quantum dot comprising a neutrally charged coating, a negatively charged coating, or a glutathione coating. In one embodiment, the at least one NP is a quantum dot that is lipophilic, organic, or a cadmium-selenide/zinc sulfide (CdSe/ZnS) quantum dot capped with octadecyl amine ligands (ODA). In one embodiment, the silica nanosphere has a diameter of about 10 nm to about 1200 nm. In one embodiment, the silica nanosphere has a diameter of about 20 nm to about 400 nm. In one embodiment, the porous silicon nanoshard has a porosity between about 20% and about 80%. In one embodiment, the porous silicon nanoshard has a diameter of about 1 nm to about 1000 nm. In one embodiment, the porous silicon nanoshard has a diameter of about 20 nm to about 400 nm. In one embodiment, the at least one NP further comprises a polymer within the NP.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts examples of porous silicon nanoshards formed from electrochemically etched porous silicon thin films.

FIG. 2 depicts a flowchart illustrating an exemplary method of synthesizing porous silicon nanoshards.

FIG. 3A and FIG. 3B depict pictures of silicon wafer and chips pre- and post-treatment. FIG. 2A depicts silicon wafer and chips. FIG. 2B depicts the porous silicon film after etching.

FIG. 4 depicts the etch cell and its components, comprising an O-ring, a platinum electrode, and an aluminum electrode.

FIG. 5 is a schematic depicting the three mass stages prior to etching (m₁), after etching (m₂), and after dissolving (m₃) for the purposes of calculating porosity and film thickness.

FIG. 6 depicts the results of experiments demonstrating the effect of etch rate on current density.

FIG. 7 depicts the results of experiments demonstrating that porosity under gravimetric analysis is a function of current density.

FIG. 8A and FIG. 8B depict SEM images of (FIG. 8A) the surface and (FIG. 8B) cross section of a porous silicon film synthesized from p-type silicon using 17.4 mA/cm² for 1200 seconds.

FIG. 9A and FIG. 9B depict porous silicon nanoshard samples after sonication and centrifugation. FIG. 9A depicts three samples after being subjected to the same 1200 second sonication treatment. FIG. 9B depicts, from left to right, the supernatant after 5000 G, 10000 G, and 15000 G centrifugation for 30 minutes.

FIG. 10 depicts (left) a sample after sonicating for 360 seconds with precipitated large particles, and (right) a sample with clear supernatant after centrifugation containing nanoshards. Dynamic light scattering (DLS) analysis shows the average size of the supernatants is about 150 nm; polydispersity index (PDI) is 0.2.

FIG. 11 depicts TEM images of PSi nanoshards produced from p-type 100 silicon wafers showing little particle aggregation and irregular shape particles having sharp edges ranging in size from 100 nm to 300 nm.

FIG. 12 depicts a UV-Vis measurement of a porous silicon nanoshard sample in pure water. The upper line is the absorbance of the nanoshards scanned from 200 nm to 400 nm. The lower line is the base line of pure water.

FIG. 13 depicts a plot showing porous silicon nanoshard degradation over time in deionized water and 0.5×PBS. The bottom two lines are native nanoshards and the top two lines are thermally oxidized nanoshards. The data shows that oxidation can stabilize the nanoshards against erosion, even in high salt solution.

FIG. 14 depicts a plot showing additional degradation of oxidized nanoshards over time. Results indicate that even after 10 days in 0.5×PBS, the nanoshard concentration has degraded less than 10%.

FIG. 15A and FIG. 15B depict results from DLS and zeta potential measurements on nanoshards produced from 10 μm thick mesoPSi thin films with differing porosity: (FIG. 15A) 69.3% and (FIG. 15B) 33.6%. Solution concentrations ˜3 mg/mL and pH=5.5-6.0. Results show near equivalent nanoshard size ˜200 nm, polydispersity index ˜0.3 and a negative charge, demonstrating that film porosity does not impact nanoshard physiochemical properties.

FIG. 16 depicts a selection of common allergens that may contribute to contact dermatitis.

FIG. 17 depicts the experiment using C57BL/6 hairless mice to test the effect of nanoparticles in the sensitization and challenge phase of the contact hypersensitivity response (CHS). The mice were sensitized on day 0 with 1) 0.05% 1-fluoro-2,4-dinitrobenzene (DNFB) alone, 2) 0.05% DNFB with negatively charged GSH coated QDs, or 3) 0.05% DNFB with positively charged PEI coated QDs, in acetone/olive oil medium (4:1 volume ratio) on the dorsal back. On day 5 both the right and left ears were pre-measured before the challenge. The ears were challenged with 1) 0.2% DNFB alone, 2) 0.2% DNFB along with combinations of differently charged QDs (negatively charged QSH-QDs or positively charged PEI-QDs). In a separate experiment, mice were sensitized to 0.05% DNFB alone and the ears were challenged with 1) 0.2% DNFB alone, 2) 0.2% DNFB along with combinations of differently charged QDs (QSH-QDs negative charge, PEI-QDs are positive charge, mPEG-QDs neutral and lipophilic QDs), or 0.2% DNFB along with combinations of other various nanoparticles that are commercially available (silica NP, gold NP, Ag NP, carbon nanotubes and titanium dioxide), testing the effects of various NPs only in the challenge phase. On day 6, 24 hours after the challenge, the ears were measured and compared to the baseline pre-measurements. During the course of treatment, the right ears always served as the 0.2% DNFB alone treated ear and the left ears were either vehicle control or 0.2% DNFB+NP treated ears. Due to variability in animal reactivity to DNFB, the swelling of DNFB+NP co-treated ears was always compared to the swelling of control DNFB ears within the same mouse.

FIG. 18 depicts the results of experiments demonstrating the effects of altering the sensitization dose on the ear swelling response. Mice were sensitized to 3 different doses—0.5%, 0.05% and 0.025% DNFB in 4:1 acetone:olive oil vehicle. The solution was pipetted on the mouse back (day 0). 5 days later the mice were challenged to 0.2% DNFB (right ear) and vehicle alone (left ear). The ear swelling response was measured 24 and 48 hours after challenge and quantified with respect to the pre-measurement value. No significant differences were observed between the treatment groups at both 24 and 48 hours. Ears exhibited scab formation around 72 hours. N=3, 2 tailed t-Test, paired with unequal variances. Scab formation was observed at the 72 hour time point.

FIG. 19 depicts the gross representation of the mouse skin sensitized to 3 different DNFB doses. Mouse skin (dorsal back) was sensitized to 3 different concentrations of DNFB. 0.5% DNFB causes a chemical burn (eschar) on the skin, which is not ideal for NP studies as the skin barrier is impaired. Titration studies indicated that a sensitization dose of 0.05% DNFB in an acetone/olive oil vehicle was sufficient to elicit the expected magnitude ear swelling response following challenge with 0.2% DNFB in C57BL/6 hairless mice without inducing an eschar that results when the mice are sensitized with the standard 0.5% DNFB dose.

FIG. 20 depicts QD properties quantified using the Malvern Zetasizer at pH 6.7.

FIG. 21 depicts a selection of QD coatings and their chemical structures.

FIG. 22 depicts the concentration of nanoparticles in the co-challenge experiments.

FIG. 23A and FIG. 23B depict the results of mice co-sensitization experiments. (FIG. 23A) Mice were sensitized to either 0.05% DNFB alone or 0.05% DNFB+GSH QD. Various combinations were tested in the challenge phase. The mice exhibited a normal ear swelling response when challenged with 0.2% DNFB alone which was comparable to the normal ear swelling levels measured with the 0.05% DNFB alone (blue bar). Mice did not exhibit any ear swelling response to GSH QD alone indicating that they were not sensitized to GSH QDs in the sensitization phase. When GSH QDs were combined with 0.2% DNFB in the challenge phase, the ear swelling response was inhibited. These mice were sensitized to DNFB, but the combination with the nanoparticle inhibited the ear swelling response in the challenge phase (0.2% DNFB+GSH-QD). (FIG. 23B) Mice were co-sensitized to 0.05% DNFB+PEI QD. They exhibited a normal ear swelling response to both 0.2% DNFB alone and co-challenge with DNFB+PEI QD indicating that the mice were sensitized to DNFB and that PEI QDs do not suppress the inflammation in the challenge phase. *p<0.05, 2-tailed t-Test, paired with unequal variances, N=3.

FIG. 24 depicts the results of experiments testing glutathione alone. Glutathione alone was applied on the co-challenge ear with 0.2% DNFB. The application did not inhibit the swelling response compared to the 0.2% DNFB treated ear.

FIG. 25 depicts the results of co-challenge experiments with methoxy PEG QDs, DHLA QDs, organic (lipophilic) QDs, and PEI QDs. Mice were sensitized to 0.05% DNFB. In the challenge phase, the right ear was exposed to 0.2% DNFB alone and the left ear was co-challenged with 0.2% DNFB+QD combinations. GSH-QDs (negative charge), DHLA-QDs (negative charge), Methoxy PEG-QDs (neutral charge) and organic QDs all inhibited the ear swelling response (p=0.01, p=0.02, p=0.07 and p=0.005 respectively). PEI QDs with a positive charge did not inhibit the ear swelling response (p=0.42). *p<0.05, 2 tailed t-Test, paired with unequal variances, N=3.

FIG. 26 depicts the gross representation of mouse ears 1 month after challenge. Mice were sensitized to 0.05% DNFB. In the challenge phase, the right ear was exposed to 0.2% DNFB and the left ear was co-challenged with 0.2% DNFB+QD combination. Methoxy PEG QDs (neutral charge), DHLA QDs (negative charge), and organic QDs all inhibited the ear swelling response (p=0.07, p=0.02 and p=0.005 respectively). PEI QDs with a positive charge did not inhibit the ear swelling response (p=0.42). These mice were followed for a month after the challenge phase. The right ear treated with 0.2% DNFB forms scabs 72 hours after challenge and the ear falls off after 4 weeks (grooming). The groups in which the QD inhibited the ear swelling response, the left ear is intact after a month. PEI QDs did not inhibit the swelling response so the left ear forms a scab as shown in the picture.

FIG. 27 depicts evidence of GSH QD presence in the mouse ear tissue obtained after co-challenge. GSH QD presence can be observed in the histology section of the ear tissue obtained after the in vivo co-challenge experiment. GSH QDs have penetrated into the cartilage region after the topical exposure (top panel). In comparison, the presence of PEI QDs is negligible in the ear tissue obtained after the PEI QD co-challenge experiment. Scale Bar=10 μm.

FIG. 28A, FIG. 28B, and FIG. 28C depict the results of experiments quantifying ex vivo penetration of quantum dots in mouse skin using confocal microscopy. GSH, PEI, methoxy PEG and organic QDs were applied on mouse skin in an ex vivo set-up in acetone/olive oil medium for a duration of 24 hours. The samples were imaged using confocal microscopy to detect QDs in the intact epidermis from 0 μm (stratum corneum) to 40 μm deep into the epidermis. (FIG. 28A) Side profile view of stacks shows that GSH, methoxy PEG and organic QDs penetrate more uniformly and deeper into the skin as compared to PEI QDs. PEI QDs are present mainly in the stratum corneum and hair follicles (visible in the streaks on the X and Y profiles). (FIG. 28B) Plot shows the penetration of QDs from 0-40 μm into viable epidermis. In the 4 treatment groups QDs are mainly concentrated in the region between 0-25 (FIG. 28C) The bar plot shows the overall QD presence in the treatment groups with organic QDs showing the highest overall penetration and PEI QD the lowest QD presence. *p<0.05, 2 tailed t-Test, unpaired with unequal variances. #p<0.05, 2 tailed t-Test, unpaired with unequal variances (organic QD group significant with respect to all other test groups), N=5.

FIG. 29 depicts top down confocal microscope views of skin sections at 10 μm depth from the stratum corneum. Ex vivo mouse skin exposed to QDs coated with different ligands was imaged using confocal microscopy. Organic-QDs penetrate to a much greater extent compared to all other treatment groups. PEI-QDs penetrate the least through the stratum corneum and were observed to be concentrated in the hair follicle areas. Scale Bar=5 μm.

FIG. 30 depicts the results of DNP assays to detect DNFB-protein adducts in ear tissue. DNP Assay run in triplicate on 0.2% DNFB treated ears (Right, marked as R1, R2, and R3) and GSH+0.2% DNFB treated ears (Left, marked as L1, L2, and L3). All tissue collected 24 hours after challenge. The co-treated ears displayed slight, non-significant reduction in adduct detection intensity as illustrated by the representative Western blot. The implication of this reduction may be due to decreased presence of DNFB-protein adducts, increased adduct clearance, or modification of the antigen decreasing antibody recognition. However, the Western blots indicate that DNFB protein adducts are present in detectable levels in both DNFB treated and DNFB+GSH QD co-treated ears. N=3.

FIG. 31A through FIG. 31D depict the results of co-challenge experiments with different NP types. (FIG. 31A) Mice were sensitized to 0.05% DNFB alone. In the challenge phase, the right ear was exposed to 0.2% DNFB and the left ear was co-challenged 0.2% DNFB+NP combination. AuNP (20 nm), Silica NP (20 nm, 50 nm, 160 nm) and AgNP (20 nm) inhibited the ear swelling response. Aminated silica NP (50 nm, positive charge), carbon nanotubes (CNT) and titanium dioxide (TiO₂) did not inhibit the ear swelling response. Silica NP (400 nm) inhibited the swelling response but inhibition was not significant compared to the control ear. CNTs and TiO₂ exacerbate the ear swelling response significantly over the control. (FIG. 31B) Mice were sensitized with 15% 2-deoxyurushiol in an acetone vehicle. The right ear was challenged with 15% 2-deoxyurushiol alone and the left ear was co-challenged with 15% 2-deoxy urushiol and silica NP 20 nm in an acetone/olive oil vehicle. Silica NPs inhibited the ear swelling response to 15-deoxyurushiol. (FIG. 31C) TiO₂ NPs did not inhibit the ear swelling response in the presence of 2-deoxyurushiol. *p<0.05, 2 tailed t-Test, paired with unequal variances, N=3-4. (FIG. 31D) Silicon nanoshards inhibited the ear swelling response to 0.2% DNFB compared to DNFB alone treated ear. *p<0.05, 2-tailed t-Test, paired, N=4.

FIG. 32 depicts the gross representation of the mouse ear 24 hours after challenge. Mice were sensitized to 0.05% DNFB. In the challenge phase, the right ear was exposed to 0.2% DNFB and the left ear was co-challenged 0.2% DNFB+Silica NP-20 nm. No swelling was observed in the co-challenged ear (Left Ear).

FIG. 33 depicts ear sections stained with H&E (Hematoxylin & Eosin Stain). H&E sections from mouse ears show inhibition of the ear swelling response in the case of GSH QDs and SiNP (20 nm) compared to vehicle ear. There are fewer cell infiltrates observed in these ear sections. A huge swelling response is observed in the 0.2% DNFB, 0.2% DNFB+CNT, 0.2% DNFB+TiO₂ treated ears. A large number of cell infiltrates can be observed in these sections. The gaps observed in the tissue sections where swelling is observed is due to edema.

FIG. 34 depicts the results of experiments applying silica nanoparticles to the challenge ear with a constant surface area. Mice were sensitized to 0.05% DNFB. In the challenge phase, the right ear was exposed to 0.2% DNFB and the left ear was co-challenged with 0.2% DNFB+silica NP combination with a constant surface area. All silica NPs (20, 50, 150, and 400 nm) except 1200 nm silica NP inhibited the ear swelling response in the challenge phase over the control (DNFB alone treated) ear (dotted line). Data is represented as fold change over the control ear. **p<0.001, *p<0.01, 2 tailed t-test paired, error bars denote SEM, N=3.

FIG. 35 depicts the results of experiments applying silica nanoparticles to the challenge ear with a constant particle number. Mice were sensitized to 0.05% DNFB. In the challenge phase, the right ear was exposed to 0.2% DNFB and the left ear was co-challenged with 0.2% DNFB+silica NP combination with a constant particle number. 400 nm silica NPs inhibit the ear swelling response over the control. Data is represented as fold change over the control (DNFB alone treated) ear (dotted line). *p<0.01, 2 tailed t-test paired, error bars denote SEM, N=3.

FIG. 36 depicts the results of experiments applying silica nanoparticles to the challenge ear with a constant mass. Mice were sensitized to 0.05% DNFB. In the challenge phase, the right ear was exposed to 0.2% DNFB and the left ear was co-challenged with 0.2% DNFB+silica NP combination with a constant mass. 20, 50, 160, 400 (not significant), and 1200 nm silica NPs inhibit the ear swelling response over the control. Data is represented as fold change over the control (DNFB alone treated) ear (dotted line). *p<0.01, 2 tailed t-test paired, error bars denote SEM, N=3.

FIG. 37 depicts the results of experiments demonstrating that DNFB adducts are reduced in ear tissue upon co-treatment with 20 nm silica nanospheres. Ear tissue from both ears treated with only 0.2% DNFB (right ear) and ears co-treated with 0.2% DNFB and 0.2 mg/mL 20 nm silica nanospheres (left ear) were homogenized in a protein extraction buffer and analyzed via Western blot. The Western blot was incubated in a primary antibody specific for dinitrophenol-protein adducts, which are identical to the adducts formed by DNFB. An HRP-linked secondary antibody and chemiluminescent substrate were used to detect the protein adducts, and a densitometer was used to quantify the intensity of the bands. A decrease in total intensity was observed in the DNFB and 20 nm silica nanosphere co-treated ear protein, compared to the DNFB treatment alone. This demonstrates that a mechanism for the nanoparticle efficacy in reducing swelling may be partial reduction in the bioavailability of DNFB in the ear. N=3, p=0.045 (ANOVA).

FIG. 38 depicts the results of experiments applying GSH QD on the mouse ear in the challenge phase in a step-wise manner. It was observed that the ear swelling response was inhibited when GSH QDs were applied 1 hour after, 3 hours after, and 1 hour prior to 0.2% DNFB application.

FIG. 39A is a schematic illustrating the events in the sensitization phase of the contact hypersensitivity response (CHS) in mice. Several cytokine/chemokine mediators and immune cells are involved in the generation of antigen-specific memory T cells.

FIG. 39B is a schematic illustrating the events in the challenge or elicitation phase of the contact hypersensitivity response (CHS) in mice. Antigen-specific memory T cells produced during the sensitization phase are recruited to the tissue to generate a full blown inflammatory response triggered by neutrophils and mast cell degranulation.

FIG. 40 depicts the results of experiments demonstrating the ear swelling response is dependent on the SiNP 20 nm/DNFB dosing sequence. SiNPs were applied on the ears either before (1, 2, and 3 hours) or after (1, 2, and 3 hours) DNFB exposure. Ear swelling measurements were made 24 hours after challenge. Pre-treatment with SiNPs 1 and 2 hours before DNFB application and post treatment 1 hour after DNFB application inhibited the ear swelling response. *p<0.05, 2-tailed Student's t-Test, paired (compared to DNFB alone treated ear—dotted line). N=3-4.

FIG. 41 depicts the results of nanoparticle wipe off experiments. Glutathione coated quantum dots (GSH-QDs) and SiNPs were applied on the ears 1 hour before DNFB exposure. The topical NPs were wiped off before DNFB application. Ear swelling measurements were made 24 hours after challenge. In both the groups, pre-treatment with NPs inhibited the ear swelling response. *p<0.05, 2-tailed Student's t-Test, paired. N=3-4.

FIG. 42A through FIG. 42D depict the results of experiments quantifying cytokines and chemokines using the Multiplexed Cytokine Analysis (Luminex). Cytokines and chemokines were quantified in the ear tissue treated with DNFB alone (right ear) and DNFB+20 nm SiNP (left ear) at 2, 12, and 24 hours post-challenge. Concentrations of (FIG. 42A) IL-1β (control=8.7 pg/mL), (FIG. 42B) IL-6 (control=4.68 pg/mL), (FIG. 42C) KC (CXCL1) (control=50.37 pg/mL) and (FIG. 42D) MIP-2 (control=71.26 pg/mL) were all lower in the co-challenged ear at the 12 and 24 hour time point. Results were analyzed using 2-way ANOVA with post-hoc Tukey analysis. *p<0.05, $p<0.05, N=4.

FIG. 43A through FIG. 43D depict the results of experiments quantifying immune cell infiltrates in the ear tissue using IHC and histology. (FIG. 43A) The results of performing Giemsa stain on the ear sections from various treatment groups are depicted (fields of view within each group at 40× magnification). (FIG. 43B) Mast cells were quantified in the ear tissue using Geimsa stain. The number of degranulated mast cells was higher in the 0.2% DNFB, PEI QD (co-challenge with 0.2% DNFB) and CNT (co-challenge with 0.2% DNFB) as compared to intact cells (black bars). The number of degranulated mast cells was lower compared to intact cells in the SiNP 20 nm treatment group. *p<0.05, 2-tailed t-test, paired with unequal variances, N=3; n=10 (FIG. 43C) Neutrophils were quantified using histology and their number was significantly lower in the 20 nm SNP treated tissue. (FIG. 43D) CD3+ T cell infiltrates were quantified using IHC and T cell infiltrates were also significantly lower in the 20 nm SiNP treated tissue. *p<0.05, 2-tailed Student's t-Test, paired. N=3, n=10 (mast cells), N=3, n=3 (neutrophils) and N=3, n=5 (CD3+ T cells).

FIG. 44 depicts the results of experiments quantifying DNFB-protein adducts using the DNP assay. DNFB-protein adducts were quantified in the ear tissue obtained from different treatment groups. No significant differences were observed between the DNFB+NP treated ear vs. DNFB alone treated ear (ANOVA), indicating that NP presence does not reduce the bioavailability of DNFB in the skin. N=3-4.

FIG. 45A and FIG. 45B depict the results of experiments quantifying immune cell infiltrates in the ear tissue using Flow Cytometry Analysis. (FIG. 45A) CD4+ T cells, CD8+ T cells and Gr-1+ neutrophils were quantified in the ear tissue treated with DNFB alone (right ear) and DNFB+20 nm SiNP (left ear) at 2, 12, and 24 hours post-challenge. T cell infiltrates were lower in the SiNP co-challenged ear compared to the DNFB treated ear at the 12 hour time point, however the differences were not significant. (FIG. 45B) Gr-1+ neutrophils were lower in the SiNP co-challenged ear at the 12 and 24 hour time point compared to the DNFB alone treated ear. N=3.

FIG. 46A through FIG. 46D depicts the results of experiments quantifying cytokines in the ear tissue using Luminex. (FIG. 46A, FIG. 46B) IFNγ levels were higher in the 0.2% DNFB alone treated ear compared to the co-challenged ear (0.2% DNFB+20 nm SiNPs) at the 12 and 24 hour time-point, however these differences were not statistically significant (p=0.18 and p=0.15, respectively). (FIG. 46C) IL-1α levels were high in both the right and the left ear tissue with no significant differences between treatment groups. The baseline concentration of IL-1α measured in the control tissue was an average of 995.33 pg/mL, which was higher than any of the treatment groups. (FIG. 46D) TNFα concentrations in the ear tissue were close to that measured in the controls around 3.7 pg/mL. The levels TNFα measured in the lymph nodes was higher compared to the ear tissue, however, there were no significant differences between the three treatment groups (2, 12, and 24 hour post-challenge).

FIG. 47 depicts a table listing the cytokines in treated and untreated mouse ear analyzed using a multiplexed luminex panel.

FIG. 48 shows the quantification of the PD-L1 expression in ear tissue analyzed using Immunohistochemistry (IHC). No significant differences observed in PD-L1 expression between different treatment groups at the 24-hour time point. Data was analyzed using ImageJ software. At least 7 fields of view per slide. No Statistical significance from one-way ANOVA, N=4, n=7-19 fields analyzed in the tissue at 40× magnification.

FIG. 49 shows the Potential Mechanisms of Action of nanoparticles in suppressing the inflammatory response in skin in response to allergens. The schematic summarizes the results of the studies conducted. The presence of Silica NPs (20 nm) along with DNFB in the challenge phase leads to a decrease in the production of pro-inflammatory mediators such as IL-1β and IL-6. A decrease was also measured in the levels of chemoattractants-CXCL1 (KC) and MIP-2. Cellular infiltrates were examined using both flow cytometry and immunohistochemistry. The presence of NPs in the challenge phase lowered the influx of both neutrophils and T cells (CD4+ and CD8+). Suppression of mast cell degranulation, a key event in the elicitation phase of this response, was also observed. Studies that were conducted to test the dosing sequence indicated that the time window of NP application is limited to 2 hours before and after allergen exposure in order to suppress the inflammatory cascade.

### DETAILED DESCRIPTION

The present invention provides nanoparticles and methods for treating and preventing skin inflammatory conditions or disorders. The conditions or disorders include allergic contact dermatitis (ACD), irritant contact dermatitis, atopic dermatitis (AD), photoallergic dermatitis, and contact hypersensitivity (CHS), as well as other conditions or disorders associated with the skin.

### Definitions

It is to be understood that the figures and descriptions of the present invention have been simplified to illustrate elements that are relevant for a clear understanding of the present invention, while eliminating, for the purpose of clarity, many other elements found in similar inventions. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As used herein, the term “composition” or “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease, or disorder, the frequency with which such a symptom is experienced by a patient, or both, are reduced.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, “nanoparticles” are particles generally in the nanoscale. Different morphologies are possible depending on the nanoparticle composition. It is not necessary that each nanoparticle be uniform in size. “Nanoparticles” encompass nanospheres, nanoreefs, nanorods, nanoboxes, nanocubes, nanostars, nanoshards, nanotubes, nanocups, nanodiscs, nanodots, quantum dots, and the like. They may be intrinsic particles or coated with bioactive ligands.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

A “therapeutic” treatment is a treatment administered to a subject who exhibits signs or symptoms of pathology, for the purpose of diminishing or eliminating those signs or symptoms.

A “preventative” treatment is a treatment administered to a subject who does not exhibit signs or symptoms of pathology, for the purpose of blocking, delaying, or diminishing the onset of those signs or symptoms.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Compositions

The present invention provides compositions for treating and preventing skin inflammatory disorders. In certain embodiments, the composition comprises one or more nanoparticles. In some embodiments, the nanoparticles are uncoated. In other embodiments, the nanoparticles are coated. For example, nanoparticles may be coated to impart a charge, or nanoparticles may be coated to alter lipophilicity.

The nanoparticles of the invention can have any suitable size. For example, the nanoparticles can have a diameter between 1 and 500 nm. In one embodiment, the nanoparticles can have a diameter between 20 and 200 nm. Nanoparticles may have a uniform shape, such as a sphere. Nanoparticles may also have irregular shapes, such as nanoshards, further described elsewhere herein. Nanoparticles may also be crystalline or amorphous. “Crystalline” as used herein and understood in the art is defined to mean an arrangement of molecules in regular three dimensional arrays. For example, nanoparticles that are crystalline include quantum dots and nanoshards made from single crystal silicon. In other aspects, the nanoparticle is semi-crystalline, which contains both crystalline and amorphous regions instead of all molecules arranged in regular three dimensional arrays. In some embodiments, the nanoparticles may form aggregates. A single type of nanoparticle may be used, or mixtures of different types of nanoparticles may be used. If a mixture of nanoparticles is used they may be homogeneously or non-homogeneously distributed. In various aspects, the nanoparticle is biodegradable or non-biodegradable, or in a plurality of nanoparticles, combinations of biodegradable and non-biodegradable cores are contemplated.

In some embodiments, the nanoparticles comprise a polymer. Non-limiting examples of polymer cores include PLGA, PLA, PGA, PCL, PLL, cellulose, poly(ethylene-co-vinyl acetate), polystyrene, polypropylene, dendrimer-based polymers, polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof.

In other embodiments, the nanoparticle material is selected from the group consisting of gold, silver, platinum, aluminum, palladium, copper, cobalt, indium, nickel, ZnS, ZnO, Sn, SnO₂, Si, SiO₂, Fe, steel, cobalt-chrome alloys, Cd, CdSe, CdS, Agl, AgBr, Hgl₂, PbS, PbSe, ZnTe, CdTe, In₂S₃, In₂Se₃, Cd₃P₂, Cd₃As₂, InAs, and GaAs.

In certain embodiments, the nanoparticles are coated with nucleic acids. The nucleic acid coatings may provide the nanoparticles with additional therapeutic effects. Non-limiting examples of nucleic acid coatings include silencing RNA, interfering RNA, RNA fragments, and the like.

In certain embodiments, the nanoparticles have a neutral charge. The charge density on the nanoparticles can be quantified by zeta potential. In certain embodiments, the neutrally charged nanoparticles of the invention may have a zeta potential from about 0 mV to 200 mV. In one embodiment, the zeta potential is from about 0 mV to 50 mV. In another embodiment, the zeta potential is from about 0 mV to 20 mV.

In certain embodiments, the nanoparticles have a neutral charge. The charge density on the nanoparticles can be quantified by zeta potential. In certain embodiments, the neutrally charged nanoparticles of the invention may have a zeta potential from about −5 mV to 5 mV. In one embodiment, the zeta potential is from about −1 mV to 1 mV. In another embodiment, the zeta potential is about 0 mV.

In certain embodiments, the nanoparticles have a negative charge. The charge density on the nanoparticles can be quantified by zeta potential. In certain embodiments, the negatively charged nanoparticles of the invention may have a zeta potential from about −200 mV to 0 mV. In one embodiment, the zeta potential is from about −50 mV to 0 mV. In another embodiment, the zeta potential is from about −20 mV to 0 mV.

In various embodiments, nanoparticles may be negatively charged by capping. For example, the nanoparticles may be capped with a reducing agent or an antioxidant. Suitable reducing agents include, but are not limited to: acetylcysteinamide, acetylcysteine, ammonium thioglycolate, bacillithiol, BDTH2, 1,2-benzenedithiol, benzyl mercaptan, bucillamine, butanethiol, tert-butylthiol, captopril, cysteamine, cysteinec dihydrolipoamide, dihydrolipoic acid, dimercaprol, 2,3-dimercapto-1-propanesulfonic acid, dimercaptosuccinic acid, 9,10-dithioanthracene, dithioerythritol, dithiothreitol, 1,1-ethanedithiol, 1,2-ethanedithiol, ethanethiol, furan-2-ylmethanethiol, gemopatrilat, glutathione, homocysteine, 3-mercapto-1-propanesulfonic acid, 3-mercapto-3-methylbutan-1-ol, 2-mercaptoethanol, 2-mercaptoindole, 4-mercaptophenylacetic acid, 3-mercaptopropane-1,2-diol, 2-mercaptopyridine, 3-mercaptopyruvic acid, methanethiol, mycothiol, omapatrilat, ovothiol, pantetheine, penicillamine, phosphopantetheine, 1,2-propanedithiol, 1,3-propanedithiol, propanethiol, rentiapril, sodium maleonitriledithiolate, thioglycolic acid, thiomalic acid, thiophenol, thiorphan, thiosalicylic acid, tiopronin, tixocortol, trypanothione, zinc pyrithione and combinations thereof.

Suitable antioxidant capping materials include, but are not limited to: ascorbic acid and its salts, ascorbyl palmitate, ascorbyl stearate, anoxomer, benzyl isothiocyanate, m-aminobenzoic acid, o-aminobenzoic acid, p-aminobenzoic acid (paba), butylated hydroxyanisole (bha), butylated hydroxytoluene (bht), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumahc acid, 3,4-dihydroxybenzoic acid, n,n′-diphenyl-p-phenylenediamine (dppd), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (edta), eucalyptus extract, eugenol, ferulic acid, flavonoids (e.g., catechin, epicatechin, epicatechin gallate, epigallocatechin (egc), epigallocatechin gallate (egcg), polyphenol epigallocatechin-3-gallate), flavones (e.g., apigenin, chrysin, luteolin), flavonols (e.g., datiscetin, myhcetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutahc acid, hydroquinone, n-hydroxysuccinic acid, hydroxytryrosol, hydroxyurea, rice bran extract, lactic acid and its salts, lecithin, lecithin citrate; r-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (ndga), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmahnic acid, sage extract, sesamol, silymahn, sinapic acid, succinic acid, stearyl citrate, syhngic acid, tartaric acid, thymol, tocopherols (i.e., alpha-, beta-, gamma- and delta-tocopherol), tocothenols (i.e., alpha-, beta-, gamma- and delta-tocothenols), tyrosol, vanilic acid, 2,6-di-tert-butyl-4-hydroxymethylphenol (i.e., ionox 100), 2,4-(ths-3′,5′-bi-tert-butyl-4′-hydroxybenzyl)-mesitylene (i.e., ionox 330), 2,4,5-thhydroxybutyrophenone, ubiquinone, tertiary butyl hydroquinone (tbhq), thiodipropionic acid, thhydroxy butyrophenone, tryptamine, tyramine, uric acid, vitamin k, vitamin q10, wheat germ oil, zeaxanthin, or combinations thereof.

In certain embodiments, the nanoparticles are lipophilic. For example, the nanoparticles may be capped with a material that is lipophilic, thereby imparting lipophilicity upon the nanoparticles. Examples of lipophilic materials include segments or groups such as: alkyl, cycloalkyl, alkenyl, alkynyl, aryl, heteroaryl, heterocyclic, halogen, and acyl groups, and polymeric aliphatic or aromatic hydrocarbons; fluorocarbons and polymers comprising fluorocarbons; silicones; hydrophobic polyethers such as poly(styrene oxide), poly(propylene oxide), poly(butylene oxide), poly(tetramethylene oxide), and poly(dodecyl glycidyl ether); and hydrophobic polyesters such as polycaprolactone and poly(3-hydroxycarboxylic acids).

An “alkyl” group refers to a saturated aliphatic hydrocarbon, including straight-chain, branched-chain and cyclic alkyl groups. In one embodiment, the alkyl group has 1-12 carbons designated as C1-C12-alkyl. In another embodiment, the alkyl group has 2-6 carbons designated as C2-C6-alkyl. In another embodiment, the alkyl group has 2-4 carbons designated as C2-C4-alkyl. In another embodiment, the alkyl group has 3-24 carbons designated as C3-C24 alkyl. The alkyl group may be unsubstituted or substituted by one or more groups selected from halogen, haloalkyl, acyl, amido, ester, cyano, nitro, and azido.

A “cycloalkyl” group refers to a non-aromatic mono- or multicyclic ring system. In one embodiment, the cycloalkyl group has 3-10 carbon atoms. In another embodiment, the cycloalkyl group has 5-10 carbon atoms. Exemplary monocyclic cycloalkyl groups include cyclopentyl, cyclohexyl, cycloheptyl and the like. An alkylcycloalkyl is an alkyl group as defined herein bonded to a cycloalkyl group as defined herein. The cycloalkyl group can be unsubstituted or substituted with any one or more of the substituents defined above for alkyl.

An “alkenyl” group refers to an aliphatic hydrocarbon group containing at least one carbon-carbon double bond including straight-chain, branched-chain and cyclic alkenyl groups. In one embodiment, the alkenyl group has 2-8 carbon atoms (a C2-8 alkenyl). In another embodiment, the alkenyl group has 2-4 carbon atoms in the chain (a C2-4 alkenyl). Exemplary alkenyl groups include ethenyl, propenyl, n-butenyl, i-butenyl, 3-methylbut-2-enyl, n-pentenyl, heptenyl, octenyl, cyclohexyl-butenyl and decenyl. An alkylalkenyl is an alkyl group as defined herein bonded to an alkenyl group as defined herein. The alkenyl group can be unsubstituted or substituted through available carbon atoms with one or more groups defined hereinabove for alkyl.

An “alkynyl” group refers to an aliphatic hydrocarbon group containing at least one carbon-carbon triple bond including straight-chain and branched-chain. In one embodiment, the alkynyl group has 2-8 carbon atoms in the chain (a C2-8 alkynyl). In another embodiment, the alkynyl group has 2-4 carbon atoms in the chain (a C2-4 alkynyl). Exemplary alkynyl groups include ethynyl, propynyl, n-butynyl, 2-butynyl, 3-methylbutynyl, n-pentynyl, heptynyl, octynyl and decynyl. An alkylalkynyl is an alkyl group as defined herein bonded to an alkynyl group as defined herein. The alkynyl group can be unsubstituted or substituted through available carbon atoms with one or more groups defined hereinabove for alkyl.

An “aryl” group refers to an aromatic monocyclic or multicyclic ring system. In one embodiment, the aryl group has 6-10 carbon atoms. The aryl is optionally substituted with at least one “ring system substituents” and combinations thereof as defined herein. Exemplary aryl groups include phenyl or naphthyl. An alkylaryl is an alkyl group as defined herein bonded to an aryl group as defined herein. The aryl group can be unsubstituted or substituted through available carbon atoms with one or more groups defined hereinabove for alkyl.

A “heteroaryl” group refers to a heteroaromatic system containing at least one heteroatom ring wherein the atom is selected from nitrogen, sulfur and oxygen. The heteroaryl contains 5 or more ring atoms. The heteroaryl group can be monocyclic, bicyclic, tricyclic and the like. Also included in this definition are the benzoheterocyclic rings. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. The heteroaryl group can be unsubstituted or substituted through available atoms with one or more groups defined hereinabove for alkyl.

A “heterocyclic ring” or “heterocyclyl” group refers to a five-membered to eight-membered rings that have 1 to 4 heteroatoms, such as oxygen, sulfur and/or in particular nitrogen. These five-membered to eight-membered rings can be saturated, fully unsaturated or partially unsaturated, with fully saturated rings being preferred. Preferred heterocyclic rings include piperidinyl, pyrrolidinyl pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, and the like. An alkylheterocyclyl is an alkyl group as defined herein bonded to a heterocyclyl group as defined herein. The heterocyclyl group can be unsubstituted or substituted through available atoms with one or more groups defined hereinabove for alkyl.

A “halogen” or “halo” group as used herein alone or as part of another group refers to chlorine, bromine, fluorine, and iodine. The term “haloalkyl” refers to an alkyl group having some or all of the hydrogens independently replaced by a halogen group including, but not limited to, trichloromethyl, tribromomethyl, trifluoromethyl, triiodomethyl, difluoromethyl, chlorodifluoromethyl, pentafluoroethyl, 1,1-difluoroethyl bromomethyl, chloromethyl, fluoromethyl, iodomethyl, and the like.

An “acyl” group as used herein encompasses groups such as, but not limited to, formyl, acetyl, propionyl, butyryl, pentanoyl, pivaloyl, hexanoyl, heptanoyl, octanoyl, nonanoyl, decanoyl, undecanoyl, dodecanoyl, benzoyl and the like.

The coating of the nanoparticle may comprise a monolayer or multilayers of lipophilic compounds, wherein the organic compounds can be small molecules, monomers, oligomers or polymers. In particular embodiments, the organic compounds are selected from the group consisting of alkylthiols, e.g., alkylthiols with C3-C24 chains, arylthiols, alkylarylthiols, alkylthiolates, ω-functionalized alkylthiolates, arenethiolates, (γ-mercaptopropyl)tri-methyloxysilane, dialkyl sulfides, diaryl sulfides, alkylaryl sulfides, dialkyl disulfides, diaryl disulfides, alkylaryl disulfides, alkyl sulfites, aryl sulfites, alkylaryl sulfites, alkyl sulfates, aryl sulfates, alkylaryl sulfates, xanthates, oligonucleotides, polynucleotides, dithiocarbamate, alkyl amines, aryl amines, diaryl amines, dialkyl amines, alkylaryl amines, arene amines, alkyl phosphines, dialkyl phosphines, aryl phosphines, diaryl phosphines, alkylaryl phosphines, dialkyl phosphines, diaryl phosphines, alkylaryl phosphines, phosphine oxides, alkyl carboxylates, aryl carboxylates, dialkyl carboxylates, diaryl carboxylates, alkylaryl carboxylates, dialkyl carboxylates, diaryl carboxylates, alkylaryl carboxylates, cyanates, isocyanates, peptides, proteins, enzymes, polysaccharides, phospholipids, and combinations and derivatives thereof.

Other organic compounds suitable as capping agents include, but are not limited to, alkenyl thiols, alkynyl thiols, cycloalkyl thiols, heterocyclyl thiols, heteroaryl thiols, alkenyl thiolates, alkynyl thiolates, cycloalkyl thiolates, heterocyclyl thiolates, heteroaryl thiolates, alkenyl sulfides, alkynyl sulfides, cycloalkyl sulfides, heterocyclyl sulfides, heteroaryl sulfides, alkenyl disulfides, alkynyl disulfides, cycloalkyl disulfides, heterocyclyl disulfides, heteroaryl disulfides, alkenyl sulfites, alkynyl sulfites, cycloalkyl sulfites, heterocyclyl sulfites, heteroaryl sulfites, alkenyl sulfates, alkynyl sulfates, cycloalkyl sulfates, heterocyclyl sulfates, heteroaryl sulfates, alkenyl amines, alkynyl amines, cycloalkyl amines, heterocyclyl amines, heteroaryl amines, alkenyl carboxylates, alkynyl carboxylates, cycloalkyl carboxylates, heterocyclyl carboxylates, heteroaryl carboxylates.

### Methods of Use

The invention relates to methods of using the nanoparticles, nanoparticle compositions, and pharmaceutical compositions of the present invention. In various embodiments, the methods relate to treating and preventing skin inflammatory disorders. The conditions or disorders include allergic contact dermatitis (ACD), irritant contact dermatitis, atopic dermatitis (AD), photoallergic dermatitis, and contact hypersensitivity (CHS), as well as other conditions or disorders associated with the skin.

The methods are useful for treating ACD and CHS caused by contact with compounds such as acrylate, bacitracin, balsam of peru, bronopol, budesonide, benzocaine, tretracaine, dibucaine, diphenyl guanidine, zinc dibutyldithiocarbamate, zinc diethyldithiocarbamate, isothiazolinone, cobalt dichloride, cocamidopropyl betaine, colophony, diazolidinyl urea, dimethyl fumarate, epoxy, resin, ethylenediamine dihydrochloride, formaldehyde, sodium thiosulfate, hydrocortisone, imidazolidinyl urea, mercaptobenzothiazole, methyldibromo glutaronitrile, dialkyl thiourea, neomycin sulfate, nickel sulfate, paraphenylenediamine, potassium dichromate, propylene glycol, quaternium, quinoline, thimerosal, tetramethylthiuram monosulfide, disulfiram, tetramethylthiuram disulfide, dipentamethylenethiuram disulfide, tixocortol-21-pivalate, lanolin, urushiol, deoxyurushiol, cinnamaldehyde, dinitrofluorobenzene, oxalozone, and the like.

The methods comprise the administration of a nanoparticle composition by any suitable method known in the art. The methods of administration permit the nanoparticle composition to be administered locally to the selected target tissue. In one embodiment, the method of administration includes injection of a solution or composition containing the nanoparticle composition. In one embodiment, the nanoparticle composition is administered in the region of the affected skin. In other embodiments, other methods of administration, such as sub-cutaneous injection, may be employed where appropriate.

In one embodiment, the method of administration includes topical application of a solution or composition containing the nanoparticle. The nanoparticle composition described herein can be incorporated into any topical formulation known in the art. Suitable compositions include, but are not limited to, creams, lotions, hydrogels, jellies, sprays, pastes, adhesives, emulsions, nanoparticles, microparticles, drops, powders, and combinations thereof.

In one embodiment, the method of administration includes topical application of a controlled release system that controllably releases the nanoparticle composition to the target tissue. In one embodiment, the controlled release system comprises an adhesive patch (as a nanoparticle composition depot) placed onto the surface of the skin of the patient, where the patch comprises a polymeric carrier which can release a therapeutically effective amount of a nanoparticle composition onto the skin surface of the patient. Application of a nanoparticle composition adhesive, polymeric patch can be preceded by pretreatment of the skin with ethanol wipes or dermal abrasion, and the patch can be used concurrently or in conjunction with a suitable permeation enhancement methodology such as iontophoresis. In one embodiment, the controlled release system comprises microneedles placed onto the surface of the skin of the patient, where the microneedles painlessly releases a therapeutically effective amount of a nanoparticle composition into the skin of the patient.

In one embodiment, the method of administration includes implantation of a controlled release system that controllably releases the nanoparticle composition to the target tissue. An implantable controlled release system reduces the need for repeat applications. Local administration of a nanoparticle composition can provide a high, local therapeutic level of the nanoparticles. A controlled release polymer capable of long term, local delivery of a nanoparticle composition to a target region of skin permits effective dosing of the nanoparticle composition.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

In the method of treatment, the administration of the composition of the invention may be for either “prophylactic” or “therapeutic” purpose. When provided prophylactically, the composition of the present invention is provided in advance of any symptom, although in particular embodiments the invention is provided following the onset of one or more symptoms to prevent further symptoms from developing or to prevent present symptoms from becoming worse. The prophylactic administration of composition serves to prevent or ameliorate any subsequent symptom. When provided therapeutically, the pharmaceutical composition is provided at or after the onset of a symptom. Thus, the present invention may be provided either prior to the anticipated exposure to a disorder-causing agent or disorder state or after the initiation of the disorder.

The compositions of the invention may be administered an hour, a day, a week, a month, or even more, in advance of an inflammatory event. Further, the compositions of the invention may be administered an hour, a day, a week, or even more, after administration of a composition of the invention, or any permutation thereof. The frequency and administration regimen will be readily apparent to the skilled artisan and will depend upon any number of factors such as, but not limited to, the type and severity of the symptoms being treated, the age and health status of the subject, the identity of the compound or compounds being administered, the route of administration of the various compositions, and the like.

### Pharmaceutical Compositions

The present invention provides pharmaceutical compositions comprising one or more nanoparticle compositions of the present invention. The relative amounts of the nanoparticle, the pharmaceutically acceptable carrier, and any additional ingredients in a pharmaceutical composition of the invention will vary, depending upon the identity, size, and condition of the subject treated and further depending upon the route by which the composition is to be administered. The formulations will also vary depending on the mass, surface area, and number of nanoparticles. By way of example, the dose per square centimeter of skin will depend on efficacy; typical mass dose/cm² may range from 1 μg/cm² to 10 mg/cm². Non-limiting example formulations are further provided in Table 1.

TABLE 1
Exemplary formulations based on treatment surface area,
particle diameter, particle count, and particle mass.

| | 20 nm | | 50 nm | | 160 nm | | 400 nm | | 1200 nm | |
|---|---|---|---|---|---|---|---|---|---|---|
| Surface Area | 3 | cm² | 3 | cm² | 3 | cm² | 3 | cm² | 3 | cm² |
| Particle # | 2.4 × 10¹¹ | | 3.8 × 10¹⁰ | | 3.7 × 10⁸ | | 5.9 × 10⁸ | | 6.6 × 10⁷ | |
| Mass | 0.002 | mg | 0.004 | mg | 0.02 | mg | 0.04 | mg | 0.004 | mg |
| Particle # | 5 × 10⁸ | | 5 × 10⁸ | | 5 × 10⁸ | | 5 × 10⁸ | | 5 × 10⁸ | |
| Surface Area | 0.006 | cm² | 0.04 | cm² | 0.4 | cm² | 2.5 | cm² | 22.6 | cm² |
| Mass | 4.5 × 10⁻⁶ | mg | 6 × 10⁻⁵ | mg | 0.002 | mg | 0.04 | mg | 0.94 | mg |
| Mass | 0.1 | mg | 0.1 | mg | 0.1 | mg | 0.1 | mg | 0.1 | mg |
| Surface Area | 1.4 × 10² | cm² | 6.5 × 10¹ | cm² | 1.7 × 10¹ | cm² | 7.03 | cm² | 2.4 | cm² |

| | | | | | |
|---|---|---|---|---|---|
| Particle # | 1.1 × 10¹³ | 8.3 × 10¹¹ | 2.1 × 10¹⁰ | 1.4 × 10⁹ | 5.3 × 10⁷ |

The formulations of the pharmaceutical compositions described herein may be prepared by any method known or hereafter developed in the art of pharmacology. In general, such preparatory methods include the step of bringing the active ingredient into association with a carrier or one or more other accessory ingredients. Said compositions may comprise additional medicinal agents, pharmaceutical agents, carriers, buffers, adjuvants, dispersing agents, diluents, and the like depending on the intended use and application.

In one embodiment, the additional accessory ingredient is a chemical penetration enhancer (CPE). CPEs increase skim permeability to enhance the transport of topically administered compounds. Non-limiting categories of CPEs include fatty acids, terpenes, fatty alcohol, pyrrolidone, sulfoxides, laurocapram, surfactants, amides, amines, lecithin, polyols, quaternary ammonium compounds, silicones, alkanoates, and the like.

Examples of suitable pharmaceutical carriers, excipients and/or diluents are well known in the art and include, but are not limited to, a gum, a starch (e g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, turmeric oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the biologically active compound of the invention, retains the biological activity. Preparations for parenteral administration may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like, in addition, the pharmaceutical composition of the present invention might comprise proteinaceous carriers, e.g., serum albumin or immunoglobulin, preferably of human origin.

In one embodiment, the carrier comprises a dermatologically acceptable vehicle. Exemplary dermatologically acceptable vehicles are well known in the art, and can include, for example, water, butylene glycol, triethanolamine, methylparaben, glycerin, titanium dioxide, polyacrylamide, hydrolyzed jojoba esters, propylene glycol, laureth-7, cetearyl ethylhexanoate, silica, glyceryl stearate, betaine, cyclopentasiloxane, dimethicone, cyclohexasiloxane, ammonium acryloyldimethyltaurate, dimethyl isosorbide, PEG-8 dimethicone, maltodextrin, xanthan gum, sodium cocyl isethionate, stearic acid, cetyl alcohol, sodiummethyl cocoyl taurate, polysorbate 60, biosaccharide gum, PPG-5-Ceteth-20, C12-C15 alkyl benzoate, zinc oxide, octinoxate, tribehenin, ozokerite, cyclomethicone, methicone, polyglyceryl-4 isosterate, or combinations thereof (US Patent Application Publication No. US2010/0260695). However, the dermatologically acceptable vehicle of the present invention is not limited to any particular ingredients or formulations. Rather, the composition comprises any suitable dermatologically acceptable vehicle known in the art or discovered in the future.

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi liquid preparations such as ionic liquids, liniments, lotions, oil in water or water in oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents, such as isopropyl alcohol, propylene glycol, or sodium xylene sulfonate, are known to those of skill in the art.

The pharmaceutical compositions provided herein may be administered as controlled-release compositions, i.e. compositions in which the active ingredient is released over a period of time after administration. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

A nanoparticle composition may be administered alone, or in combination with other drugs and/or agents as pharmaceutical compositions. The composition may contain one or more added materials such as carriers and/or excipients. As used herein, “carriers” and “excipients” generally refer to substantially inert, non-toxic materials that do not deleteriously interact with other components of the composition. These materials may be used to increase the amount of solids in particulate pharmaceutical compositions, such as to form a powder of drug particles. Examples of suitable carriers include water, silicone, gelatin, waxes, and the like.

Examples of normally employed “excipients,” include pharmaceutical grades of mannitol, sorbitol, inositol, dextrose, sucrose, lactose, trehalose, dextran, starch, cellulose, sodium or calcium phosphates, calcium sulfate, citric acid, tartaric acid, glycine, high molecular weight polyethylene glycols (PEG), and the like and combinations thereof. In one embodiment, the excipient may also include a charged lipid and/or detergent in the pharmaceutical compositions. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN® surfactants (Atlas Chemical Industries, Wilmington, Del.), polyoxyethylene ethers, for example, Brij®, pharmaceutically acceptable fatty acid esters, for example, lauryl sulfate and salts thereof (SDS), and the like. Such materials may be used as stabilizers and/or anti-oxidants. Additionally, they may be used to reduce local irritation at the site of administration.

The pharmaceutical compositions may also contain a variety of active agents known in the art such as skin lightening agents, skin pigmentation darkening agents, anti-acne agents, sebum modulators, shine control agents, anti-microbial agents, anti-fungals, anti-inflammatory agents, anti-mycotic agents, anti-parasite agents, external analgesics, sunscreens, photoprotectors, antioxidants, keratolytic agents, detergents, surfactants, moisturizers, nutrients, vitamins, energy enhancers, anti-perspiration agents, astringents, deodorants, hair removers, firming agents, anti-callous agents, and agents for hair, nail, or skin conditioning.

In at least one embodiment, the composition is formulated in a liquid form. In certain embodiments, the liquid formulation of the composition allows for the nanoparticles to be stably maintained under a refrigerated or high temperature condition with the use of neither animal-derived protein, such as albumin or gelatin, as a stabilizer for botulinum toxin nor polar or acidic amino acids such as glutamine, glutamic acid, asparagine or aspartic acid.

In at least one embodiment, the composition is formulated in a lyophilized form. In certain embodiments, the lyophilized formulation of the composition allows for maintaining nanoparticle structure and achieving remarkably superior long-term stability even under high-temperature conditions which might occur during storage, transportation, or use of the nanoparticles.

### Kits of the Invention

The invention also includes a kit comprising compounds useful within the methods of the invention and an instructional material that describes, for instance, the method of administering the nanoparticles and compositions as described elsewhere herein. The kit may comprise formulations of a pharmaceutical composition comprising the active ingredient combined with a pharmaceutically acceptable carrier, such as sterile water or sterile isotonic saline. The kit may comprise injectable formulations that may be prepared, packaged, or sold in unit dosage form, such as in ampules or in multi dose containers containing a preservative. The kit may comprise formulations including, but not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a kit, the active ingredient is provided in dry (i.e., powder or granular) form for reconstitution with a suitable vehicle (e.g., sterile pyrogen free water) prior to administration of the reconstituted composition.

The kit may comprise pharmaceutical compositions prepared, packaged, or sold in the form of a sterile aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

In certain embodiments, the kit comprises instructional material. Instructional material may include a publication, a recording, a diagram, or any other medium of expression which can be used to communicate the usefulness of the device or implant kit described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Porous Silicon Nanoshards

Porous Silicon (PSi) is produced by electrochemical anodization etching of single crystal silicon with a hydrofluoric acid electrolyte solution (Sailor. M. J. Porous Silicon in Practice. New York, N.Y., John Wiley & Sons, 2012, 27; Canham, L. T. Properties of porous silicon. EMIS datareviews series. 1987, 62-76). Porous silicon is classified by the width of the pores that are formed: microporous (less than 2 nm), mesoporous (between 2 nm and 50 nm) or macroporous (over 50 nm). Porosity and pore diameter are two important properties of this material that can be exploited for drug loading and delivery. Pore diameter and porosity can be tuned by controlling the electrochemical etch parameters, such as silicon crystal orientation, doping type and level, etchant composition, and current density (Canham, L. T. Advanced Materials, 1995, 12:1033; Qin Z T et al. Particle and Particles Systems Char, 2014, 31:252-256). Porosity and etch film thickness can be measured using simple mass gravimetric technique. One characteristic of the as-etched porous silicon material is that the surface atoms are hydride terminated. This leaves the porous silicon surface hydrophobic and prone to air oxidation. Particularly for mesoporous silicon (mesoPSi) synthesized from p-type silicon, corrosion in high salt physiologic solutions is rapid. Over time, exposure to air will oxidize the surface, making it hydrophilic and less sensitive to erosion. The degradation rate can be further slowed by thermal oxidation which imparts thicker stabilizing surface oxide or by chemical means including silanation or hydrosilylation. Surface coatings can be further modified to tether target homing ligand and/or therapeutics (Wang M. Langmuir, 2015, 31(22):6179-85). The degree of surface oxide and the surface composition can be designed to tailor the PSi erosion and drug delivery rate. Drugs can be loaded within and tethered to the porous matrix to achieve a rapid burst release followed by a sustained release linked to matrix degradation.

The use of PSi films and PSi NPs for biomedical applications has grown tremendously over the past decade (Sailor. M. J. Porous Silicon in Practice. New York, N.Y., John Wiley & Sons, 2012, 27; Secret E. Advanced Healthcare Materials, 2013, 718-735; Sun W. and Puzas J. E. Adv Mater, 2007, 19:921-924; Toni M. A. et al. Biomaterials, 2014, 35(29):8394-405; Hartmann K I et al. J Ocul Pharmacol Ther, 2013, 29(5):493-500). However, the synthesis of PSi NPs from electrochemically etched PSi films has only been recently explored (Qin Z T et al. Particle & Particle Systems Characterization, 2014, 31(2):252-256; Park J H et al. Nature Materials, 2009, 331-336; Secret E et al. Advanced Healthcare Materials, 2013, 718-735; Ryu J K et al. Journal of Nanoscience and Nanotechnology, 2013, 13(1):157-160; Gu L et al. Nat Commun, 2013, 4:2326).

Compared to other types of NPs used in drug delivery applications, PSi has many advantages as the porosity can be easily altered by the synthesis protocol. Hence, dependent properties including bioactivity and drug loading can be tailored. Gold and many other types of metal and metal oxide NPs are very stable carriers that do not readily biodegrade and have a limited surface area to load drugs. If the body does not efficiently clear the NPs, there is a long term nanotoxicity concern (El-Ansary A and Al-Daihan S. J Toxicol, 2009, 2009:754810). PSi degrades into silicic acid, a natural product can be removed by kidneys (Bekersky I et al. J Pharmacol Exp Ther, 1980, 212(2):309-14). Hence, the use of PSi NPs dramatically reduce the risk of the toxic product formation and systemic metal accumulation while allowing efficient means to tailor drug loading capacity and drug delivery rate (Chiappini C and Tasciotti E. Chem Phys Chem, 2010, 11:1029-1035). Furthermore, PSi is an optical material, and its refractive index and extinction coefficient can be used to characterize its degradation rate using UV-Vis and reflection spectroscopies (DeLouise L A and Miller B L. Proceedings of SPIE, 2004, 5357:111-125).

### Nanoshard Synthesis

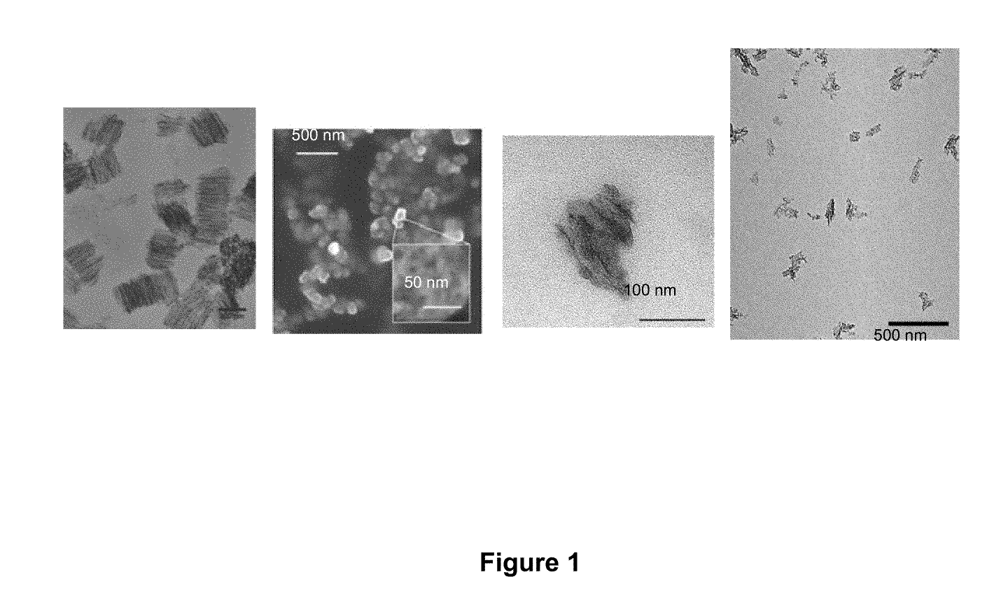

Various methods have been used to produce PSi NPs from electrochemically etched PSi films that principally utilize fracture sonication to produce particles and centrifugation or membrane filtration to isolate the nanoscale material. Despite the commonality in the protocols used, the reported shapes and sizes of particles produced vary over a wide range (FIG. 1). This likely results from fracturing protocol and post-processing effects. To those skilled in the art, it is understood that NPs may be incubated in deionized water for ˜2 weeks, ozone treated or thermally oxidized at high temperature (˜800° C.) for minutes to form an oxide layer and/or to activate their luminescence properties (Park J H et al. Nature Materials, 2009, 331-336). Alternatively, the PSi films may be degassed for 10 to 30 minutes under a nitrogen stream prior to ultrasonication for a period of minutes to 16 hours depending upon the sonication power as a means to hinder oxidation (Secret E et al. Advanced Healthcare Materials, 2013, 718-735.

A rapid synthesis protocol was developed that produces irregularly shaped PSi particles from mesoPSi films. These particles, called nanoshards, have ragged edges and range in size from <100 nm to over 500 nm. The nanoshards have properties that are ideally suited for transdermal drug delivery (TDD) applications. Although TDD has proven to be an effective means to deliver drugs systemically due to the nature of the stratum corneum barrier (the outmost layer of skin), only a small subset of low molecular weight (<500 MW) and generally hydrophobic drugs can be delivered through skin (Pauedel K S et al. Ther Deliv, 2010, 1(1):109-131). Physical means to disrupt the skin barrier, including the use of microneedles, has enabled the systemic delivery of higher molecular weight and more hydrophilic drugs through skin (Ita K. Pharmaceutics, 2015, 7(3):90-105); Vitorino C et al. Curr Pharm Des, 2015, 21(20):2698-712). Various types of nanocarriers, especially those targeting the follicular drug delivery route, have been developed as effective TDD systems (Khan N R et al. Curr Pharm Des, 2015, 21(20):2848-66; Rancan F and Vogt A. Ther Deliv, 2014, 5(8):875-7). Nanoshards may comprise an efficient TDD system. By acting like sharp shards of glass that easily slice through skin, nanoshards offer the possibility to facilitate delivery of higher molecular weight and hydrophilic drugs through skin by generating nanocuts in the skin barrier as they are massaged onto the skin surface. In addition to taking advantage of the physical properties, PSi nanoshards compared to other widely used drug carriers like gold and silver NPs, are non-toxic and biodegradable. A large surface area makes nanoshards ideal for drug loading and delivery through burst and bioerosion mechanisms.

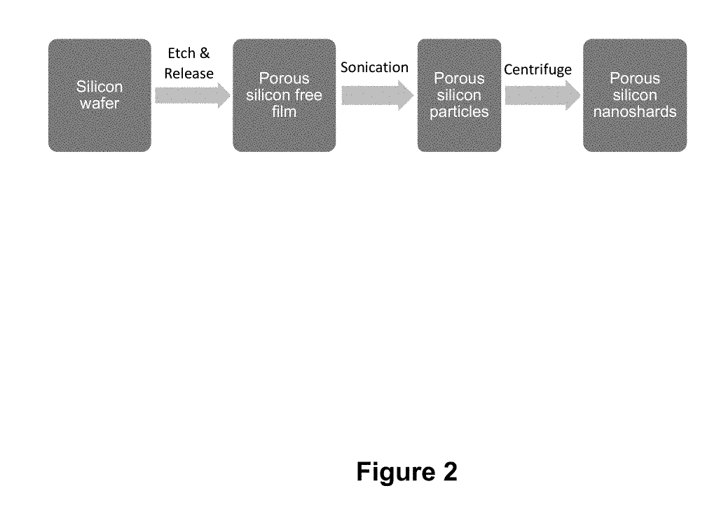

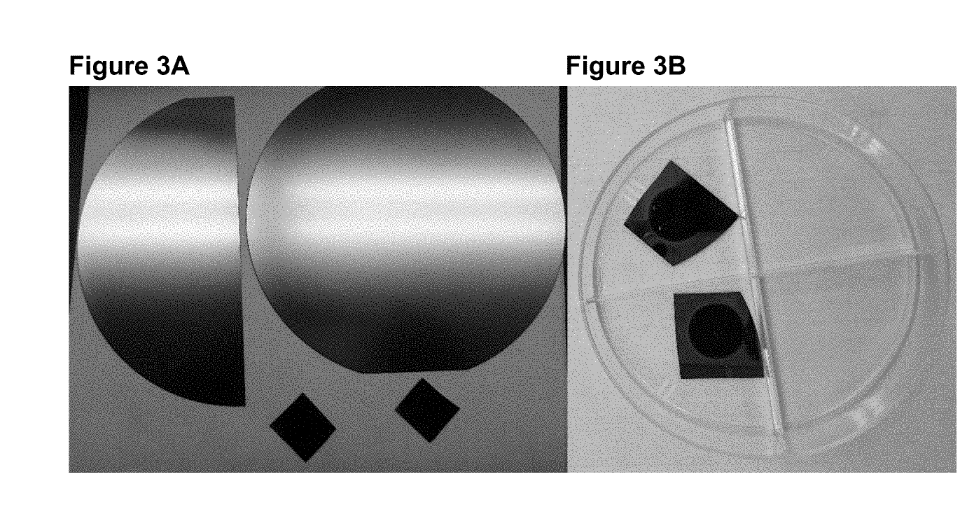

The nanoshard synthesis protocol is delineated in FIG. 2 and can be used with P-type or N-type silicon wafers with various doping levels and crystal orientation, but preferably with p-type or n-type (100) wafers with 0.01 ohm-cm resistivity (FIG. 3A and FIG. 3B). The synthesis process steps include electrochemical etching (with or without thermal oxidation), ultrasonication, centrifugation, and characterization. The material is characterized using dynamic light scattering, transmission electron microscopy, scanning electron microscopy, and ultraviolet-visible spectroscopy.

### Electrochemical PSi Film Synthesis and Release

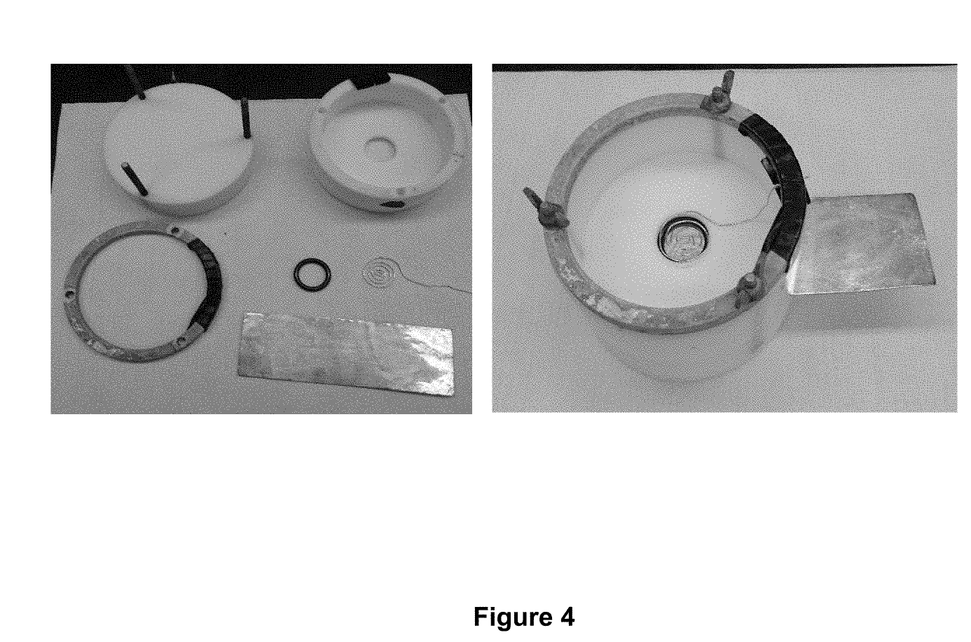

The electrochemical etch cell used to synthesize PSi films is depicted in FIG. 4 disassembled and assembled with a silicon chip prepared for etching. It is made from Teflon to resist HF corrosion. The cathode spiral is made from platinum and the anode is made from a conducting metal like silver foil. The silicon chip is sandwiched between a Viton rubber O-ring and the anode and the electrolyte are poured into the etch container. Current is applied through the silicon wafer to etch porous silicon. The etch rate, porosity, and pore size are dependent on wafer type, current density, and electrolyte composition. Table 2 summarizes the etching parameters for p-type silicon used in this work.

TABLE 2
Summary of Etch Parameters used to make mesoporous PSi Films

| | | Current | | |
|---|---|---|---|---|
| | | density | Etch rate | Porosity |
| Wafer | Electrolyte | mA/cm² | Nm/sec | % |
| p-type | 15% HF | 9 | 10 | 62 |
| 0.01-0.02 | 70% Ethanol (95%) | | | |
| ohm-cm | 15% water | | | |
| p-type | 15% HF | 17 | 15 | 65 |
| 0.01-0.02 | 70% Ethanol (95%) | | | |
| ohm-cm | 15% water | | | |
| p-type | 15% HF | 35 | 25 | 72 |
| 0.01-0.02 | 70% Ethanol (95%) | | | |
| ohm-cm | 15% water | | | |
| p-type | 15% HF | 113 | 68 | >100 |
| 0.01-0.02 | 70% Ethanol (95%) | | | electropolish |
| ohm-cm | 15% water | | | |

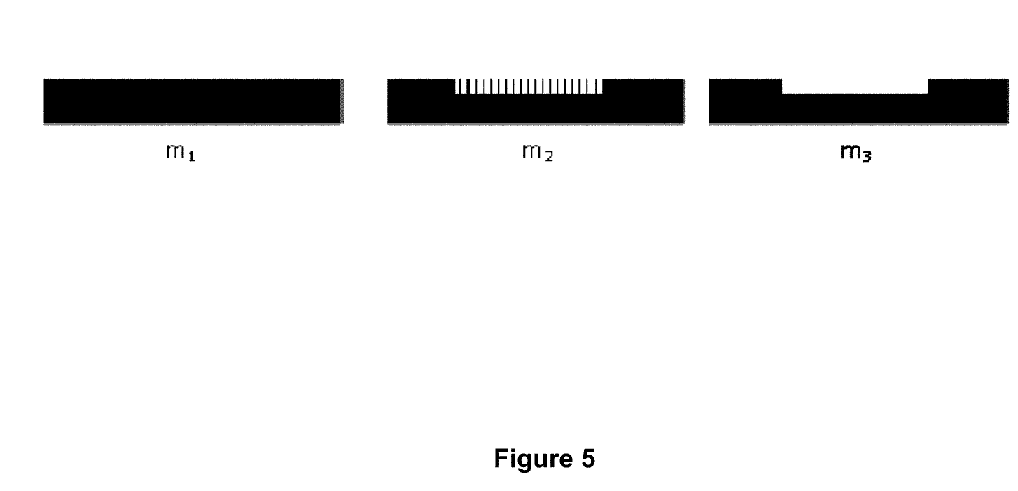

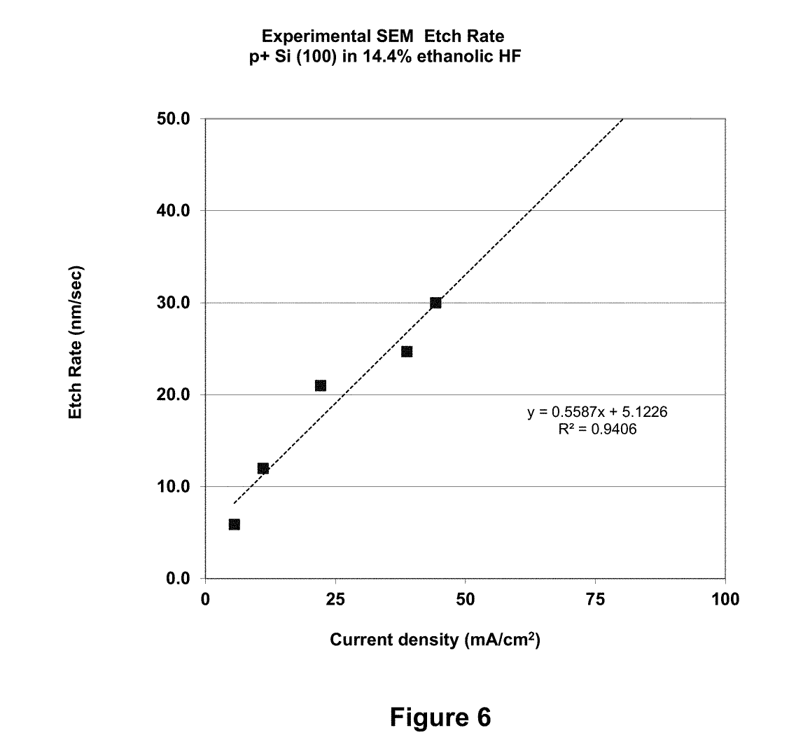

The concentration of HF mainly affects the etch rate, current density mainly affects the porosity, and pore size is mainly determined by doping level and type (Foll H et al. Materials Science and Engineering R, 2002, 280:1-49). The active etch front always occurs at the PSi-silicon wafer interface due to charge depletion and lack of electrical conduction in the porous film (Canham L T. EMIS datareviews series, 1987, 62-76; Halimaoui A. 1997, “Porous silicon formation by anodization”, in Properties of Porous Silicon. Canham, L. T., Institution of Engineering and Technology, London, ISBN 0-85296-932-5 pp. 12-22). For a given etchant composition and wafer type, a simple gravimetric method can used to estimate the porosity, film thickness, and etch rate as a function current density. The mass of the wafer chip (m₁) is measured prior to etching (FIG. 5). After etching the PSi film, the mass is remeasured (m2) to quantify the silicon removed. After dissolving the porous silicon film in a strong base (1% KOH), the silicon chip is weighed (m₃) and the porosity (P %) and film thickness (d) are calculated using Equations 1 and 2 below, where ρ is the density of silicon (ρ=2.33 g/cm³) and S is the etched area (cm²). Etch rate is calculated by dividing the film thickness by the etch time.

$$\begin{matrix}Porosity ( \% ) = \frac{{m}_{1} - {m}_{2}}{{m}_{1} - {m}_{3}} \times 100 \% & ( Equation 1 ) \\ d = \frac{m 1 - m 3}{S ρ} & ( Equation 2 )\end{matrix}$$

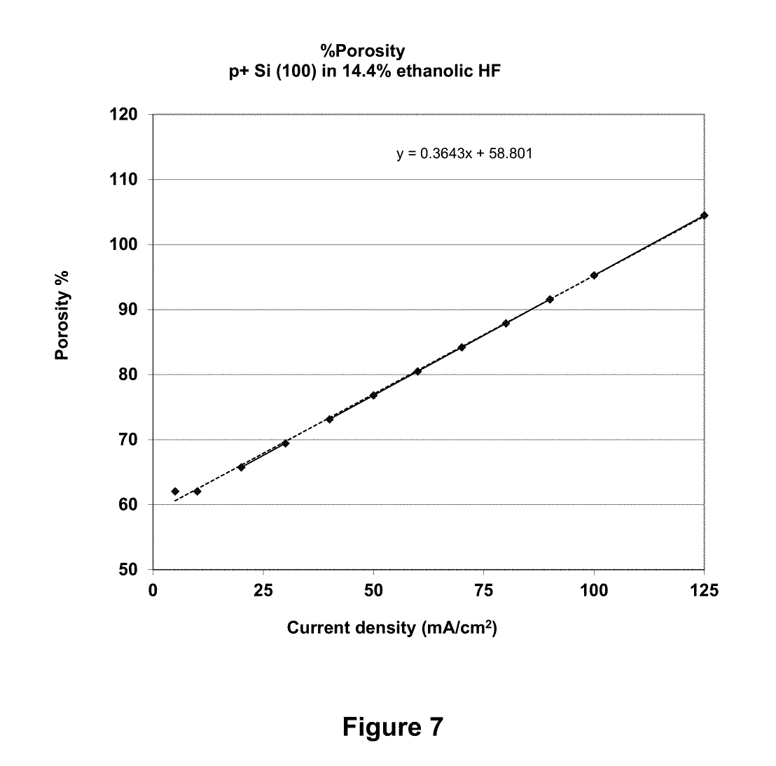

The dependence of etch rate and porosity on current density for p-type silicon (100), 0.01 ohm-cm etching are shown in FIG. 6 and FIG. 7, respectively

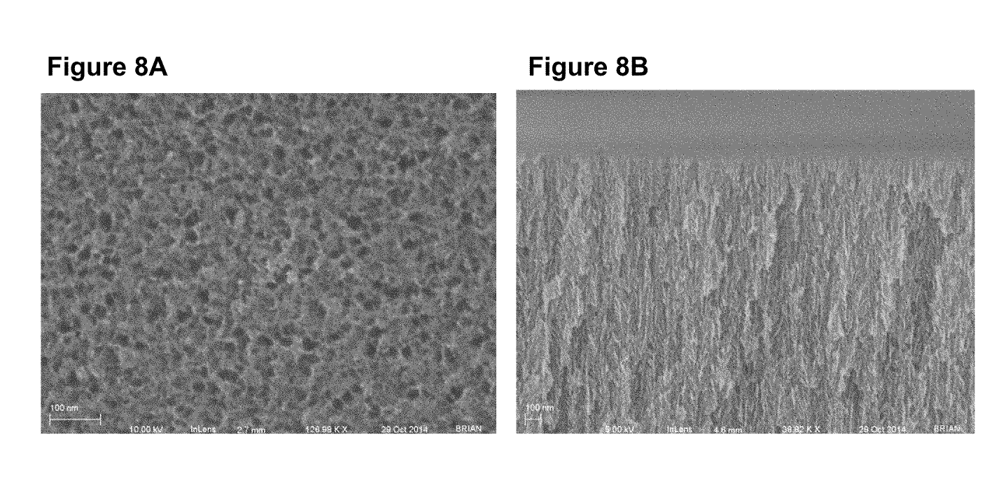

It can be seen in FIG. 7 that at a current density above ˜115 mA/cm², the predicted porosity is >100% at which the electropolishing limit has been attained. This feature can be used to release the PSi film by applying 2 or 3 current pulses >120 mA/cm² for 1 to 3 seconds each. This erodes the interface and releases the PSi film. For most of the work presented here, p-type silicon wafer (boron doped resistivity 0.01-0.0.2 ohm-cm) was etched at a constant current density ˜17 mA/cm² (etch rate=15 nm/sec) for 1200 seconds in an aqueous HF to produce a ˜18 μm thick film with 65% porosity. A SEM (Zeiss Cross Beam FIB-SEM operated between 5 and 20 KV, using chamber and in-lens detector) image of the porous film surface and cross section are shown in FIG. 8A and FIG. 8B. Results show that pores randomly nucleate on the wafer surface and exhibit a typical pore diameter between 10 and 30 nm. Other etching conditions were used to test the effect of nanoshard formation on PSi film porosity and thickness as discussed later.

### Ultrasonication and Centrifugation

To synthesize nanoshards, the freestanding PSi film was first rinsed with ethanol several times to remove any residual HF. The film was collected into a 1.5 mL Eppendorf tube and 1 mL of water was added. The high energy applied during ultrasonication causes the solution to heat up rapidly, so the tube was immersed in an ice bath. Probe ultrasonication was typically performed at a frequency of 20 kHz with a 50% duty cycle typically for 6 minutes total (360 seconds) in 1 minute intervals and ˜30 seconds rest on ice between each sonication. The application of sound energy and particle beating against each other causes the fracturing of the material that produces a heterogeneous size distribution. The size and concentration of particles produced varies from a few nanometers to several microns depending on the time and power of sonication. The higher the power and longer the sonication time, the higher the concentration and the smaller the nanoshards produced. Large particles simply settle out and centrifugation is used to remove all but the nanoparticles. Particle size is determined by dynamic light scattering and TEM measurements discussed below.

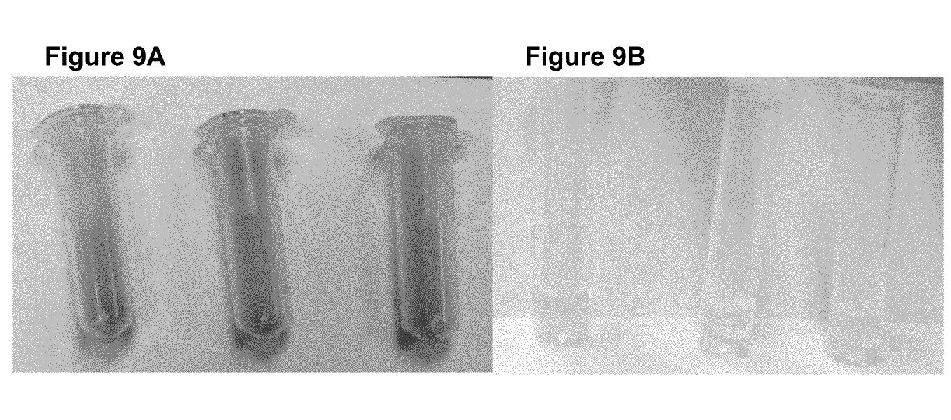

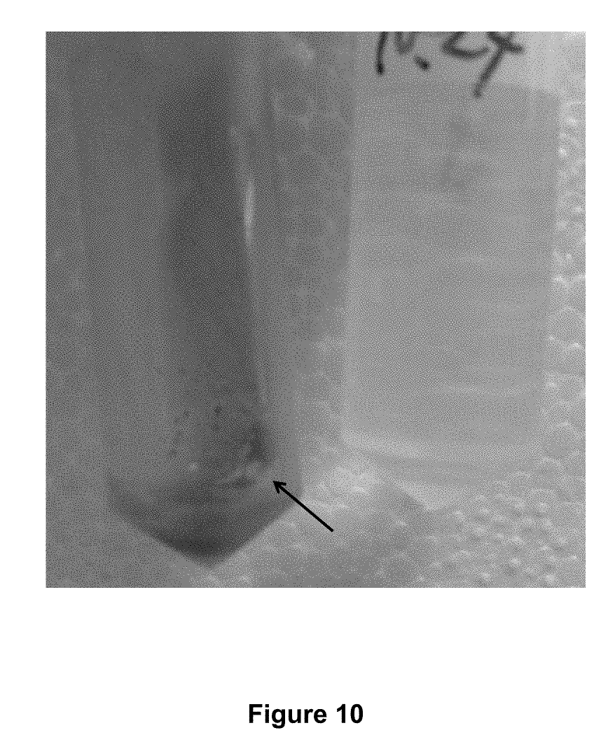

The nanoscale material was isolated and concentrated using centrifugation. First, the centrifugation speed to pellet large particles was determined. After sonication, a typical sample before centrifugation is shown in FIG. 9A. After sonication the solution appear brownish and very large particles simple fall out of solution. Samples were centrifuged at 5,000 g, 10,000 g, and 15,000 g for 30 min at 8° C. (FIG. 9B, from left to right). It can be seen that the supernatant retains a yellowish color for spinning at 5,000 g, whereas the supernatant is clear for the other two samples. A close up view of a sonicated sample before centrifugation and recovery of the supernatant after centrifugation at 10 Kg for 30 min at 8° C. is shown in FIG. 10.

### Nanoshard Characterization—Size, Charge, and Concentration

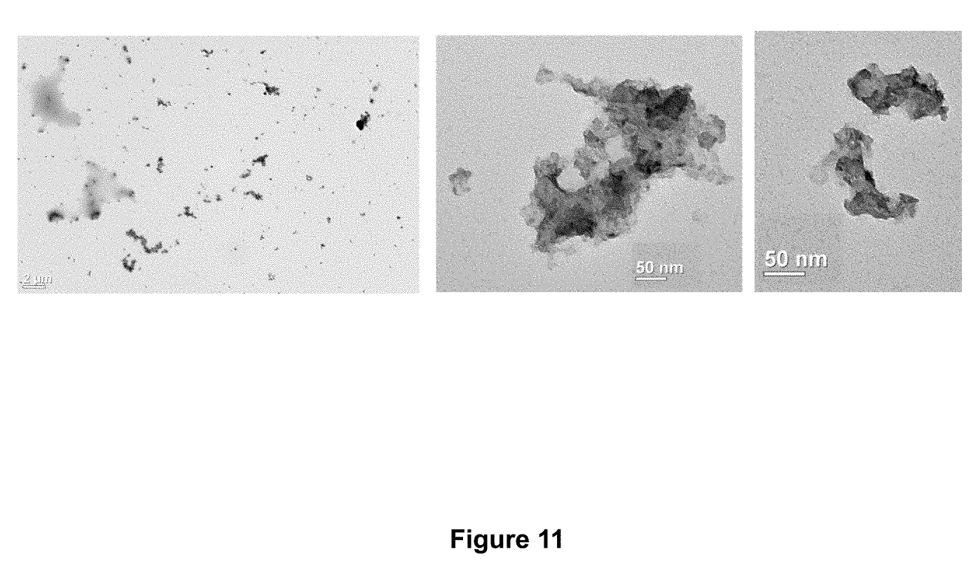

After centrifugation, the supernatant containing nanoshards is collected and analyzed for particle size and concentration using TEM, DLS, and UV-Vis spectroscopy. The DLS data shows that the average size of sample centrifuged at 5,000 g is ˜800 nm, whereas the size of the particles in samples centrifuged at 10,000 g and 15,000 g ranges between 150 nm to 200 nm. Transmission electron microscopy (TEM) is a direct way to examine how the nanoparticles appear in size, shape, and agglomeration. TEM images for particles in solution following 10,000 g centrifugation for 30 minutes is shown in FIG. 11. Results show highly dispersed irregularly shapes particles with sizes ranging from 100 to 300 nm in diameter.

The mean particle size, size distribution, and Zeta potential were measured by dynamic light scattering (DLS; Malvern Instruments). The particle size of porous silicon nanoshards was 140±30 nm and the poly dispersity index was typically 0.2 to 0.3, demonstrating a moderate size variability. Those skilled in the art will know that an index<0.05 is rarely measured except with highly monodisperse particle standards and index values greater than 0.7 indicate that the sample has a very broad size distribution and is not suitable for DLS measurements. The zeta potential is a key indicator of the stability of colloidal dispersions. The magnitude of the zeta potential indicates the degree of electrostatic repulsion between adjacent, similarly charged particles in dispersion. The zeta potential of porous silicon nanoshards measured in the water solution with pH 5.5 to 6.0 was −20±5 mV, which for those skilled in the art, is understood to be sufficient to maintain high colloidal stability.

Ultraviolet-visible spectroscopy (UV-Vis) absorption spectroscopy was used to measure the concentration of nanoshards in solution. Here, light in the visible and adjacent near-UV and near-infrared ranges is incident on a sample and the amount of light absorbed is measured as a function of wavelength. The Beer-Lambert law is used to determine the concentration of a species in solution by measuring the magnitude of the UV-Vis absorbance. The Beer Lambert law is:

A=log₁₀(I₀/I)=ϵcL

In this equation, A is the absorbance which can be measured with the UV-Vis spectroscopy, ε is a constant known as the molar absorptivity or extinction coefficient with the units of

$$\frac{1}{M * cm} ,$$

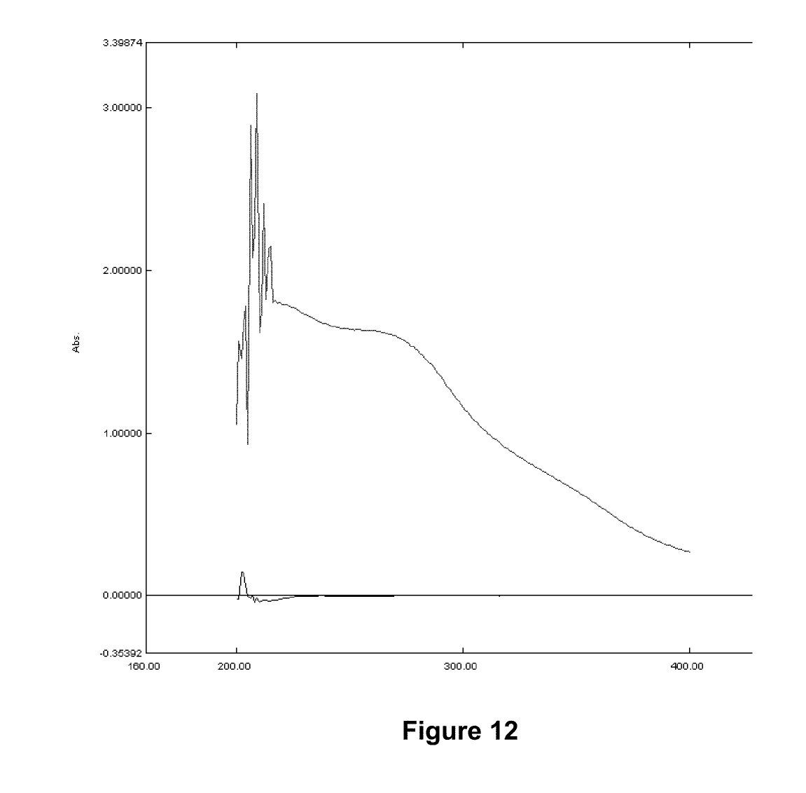

L is the path length through the sample, and c is the concentration of the species in solution. With constant ε and L, the concentration c can be calculated after measuring A with knowledge of ε. The absorbance spectrum as a function of the wavelength for nanoshards is shown in FIG. 12. The nanoshards had an absorption peak at 280 nm. The extinction coefficient of silicon at 280 nm was 5.30399. The path length was constant at 1 cm. The synthesis protocol outlined typically yielded nanoshard concentrations of ˜3 mg/ml.

### Stability of Nanoshards in Solution—Effect of PSi Film Oxidation

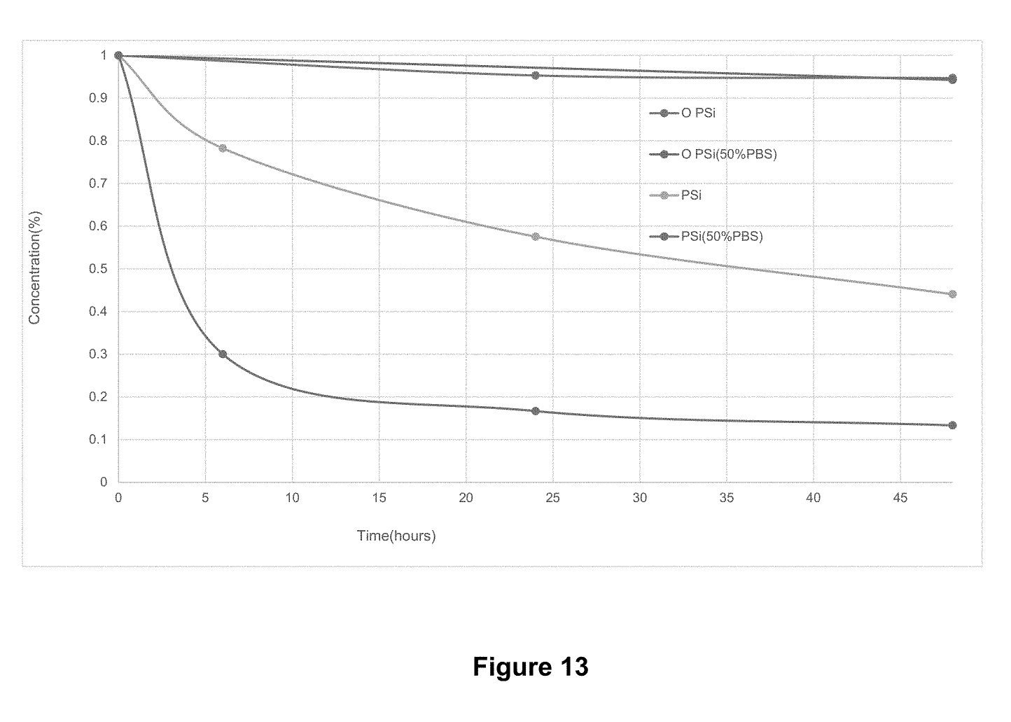

As mentioned earlier, the as-etched mesoPSi surface is hydride-terminated, hydrophobic, and prone to air oxidation and corrosion in in vitro physiologic solutions and in vivo. Thermal oxidation is a means to delay salt corrosion and tailor drug release (Borisova D et al. ACS Nano, 2011, 5(3):1939-46; Hon N K et al. J Biomed Mater Res A, 2012, 100(12):3416-21. Here, the effect of thermal oxidation on mesoPSi degradation was tested in DI water and buffered saline (PBS). After etching, the mesoPSi film was thermal oxidized before sonication in a tube furnace under pure oxygen flow for 10 min at 800° C. After oxidation, the nanoshards were prepared as outlined in FIG. 2. After synthesis, the nanoshard solution was diluted at 50% in pure DI water or in 0.5×PBS (75 mM NaCl). The degradation (decrease in nanoshard concentration) was monitored by UV-Vis. Results shown in (FIG. 13) demonstrate that nanoshards degrade rapidly in water and degrade even faster in 0.5×PBS. At 6 hours, the concentration in water decreased ˜20% whereas in 0.5×PBS it decreased to 70%. For the thermally oxidized nanoshards, there is little measurable degradation (<5%) even after 48 hrs in water or in 0.5×PBS. Moreover, the nanoshard concentration remains over 90% for 10 days (FIG. 13).

### The Effect of Nanoshard Size and Charge on PSi Film Parameters

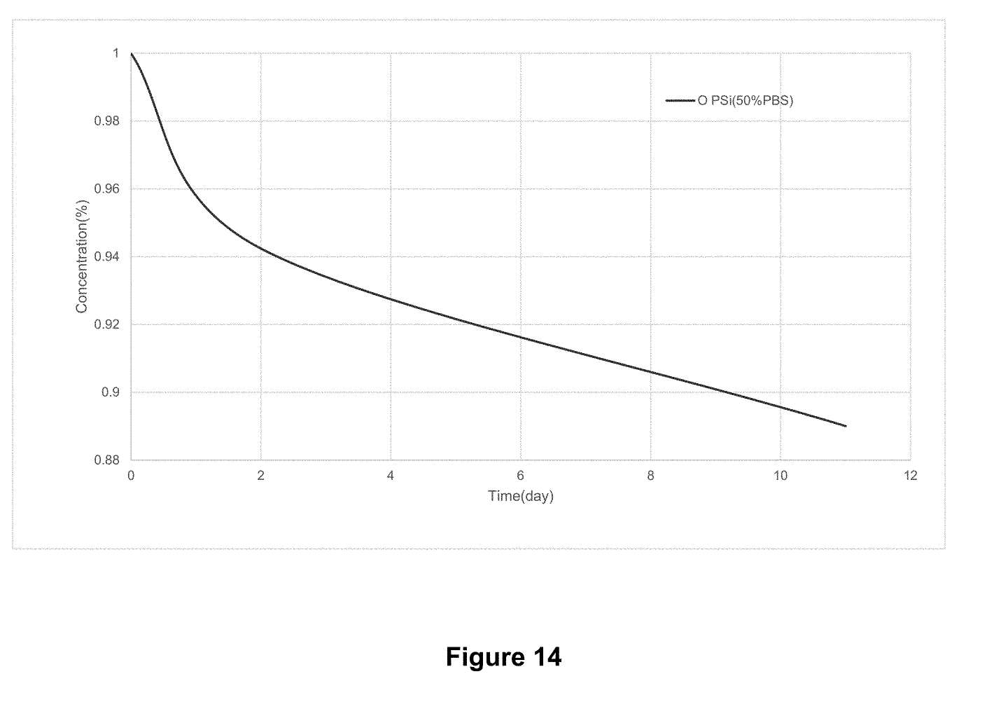

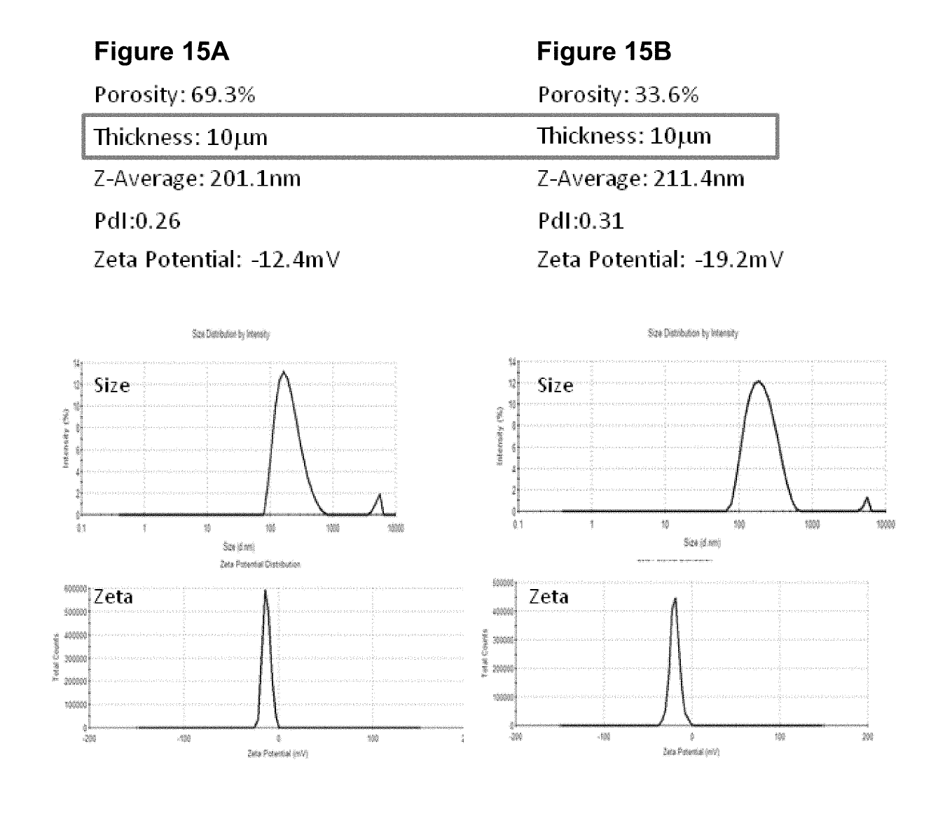

The above results discussed were obtained on nanoshards synthesized from a mesoPSi film ˜18 μm thick with 65% porosity. It is of interest to investigate the effect of nanoshard particle size and charge as a function of the PSi film thickness and porosity employing a constant sonication and centrifugation protocol. First, the effect of film porosity was investigated while keeping the film thickness constant. Two 10 μm thick mesoPSi films were produced; one with porosity of 33.6% and the other with porosity of 69.3%. Nanoshards were synthesized from these films using the same protocol described above and characterized. Results (FIG. 14) show nearly equivalent nanoshard size ˜200 nm in diameter, polydispersity index (PDI) ˜0.3, and negative charge, demonstrating that film porosity does not impact the nanoshard physiochemical properties. Next, the effect of mesoPSi film thickness (2 μm vs. 20 μm) on nanoshard formation was investigated while keeping the film porosity constant (45.7%). Results (not shown) for the nanoshard solution produced from the 20 μm film (˜3 mg/ml) found a particle size of 181 nm, a PDI=0.28, and a zeta potential of −13.4 mV, which was similar to the nanoshards produced from the 10 μm thick film (FIG. 14). However, results for the nanoshard solution produced from the 2 μm thick (˜0.3 mg/ml) film found much larger particles (681 nm) with a very high polydispersity (PDI=0.54) and an odd positive surface charge (FIG. 15A and FIG. 15B). This demonstrates that the amount of PSi material loaded in the tube during sonication and the process of particles beating against each other can affect the degree of fracturing for a fixed sonication time. To prove this, four separate 2 μm thick films were etched and combined in one tube for sonication. The negatively charged (−26.2 mV) nanoshards produced from this sample were 153 nm in diameter with a PDI=0.22, confirming that the physiochemical properties (size, size distribution, and zeta potential) of nanoparticles produced by fracture sonication are dependent on the process of particles beating against each other during sonication.

### Example 2

### Immunomodulatory Effects of Nanoparticles on the Contact Hypersensitivity Response

Allergic Contact Dermatitis (ACD) is a delayed type IV inflammatory response to sensitizers that contact skin causing pruritus, erythema and vesiculation (Krasteva, M. et al. Eur J Dermatol, 1999, 9:144-159; Krasteva, M. et al. Eur J Dermatol, 1999, 9:65-77; Mowad, C. M. Current opinion in allergy and clinical immunology, 2006, 6:340-344; Kaplan, D. H. et al. Nat Rev Immunol, 2012, 12:114-124; Cashman, M. W. et al. Dermatologic clinics, 2012, 30:87-98). Approximately 15-20% of the US population suffers from ACD. It accounts for 95% of reported occupational skin disease and is the third most common reason patients visit a dermatologist (Clark, S. C. & Zirwas, M. J. Dermatologic clinics, 2009, 27:365-383). Common contact sensitizers include nickel, latex, and urushiol in poison ivy (Bordel-Gomez, M. T. et al. Actas dermo-sifiliograficas, 2010, 101:59-75). ACD is treated with topical anti-inflammatory steroids but with limited efficacy (Usatine, R. P. & Riojas, M. American family physician, 2010, 82:249-255). In this study, an in vivo mouse model of allergic contact hypersensitivity (CHS) is used to investigate how nanoparticles may alter the CHS response. Certain nanoparticles, particularly negatively charged 20 nm silica, have been discovered to exhibit a remarkable and unexpected immunosuppressive effect upon simultaneous skin contact with dinitrofluorobenzene, a common chemical sensitizer. Silica nanoparticles also inhibit the CHS response to 2-deoxyurushiol, the chemical analogue of urushiol found in the poison ivy plant. These findings demonstrate an opportunity to develop topical therapeutics containing nanoparticles to suppress ACD.

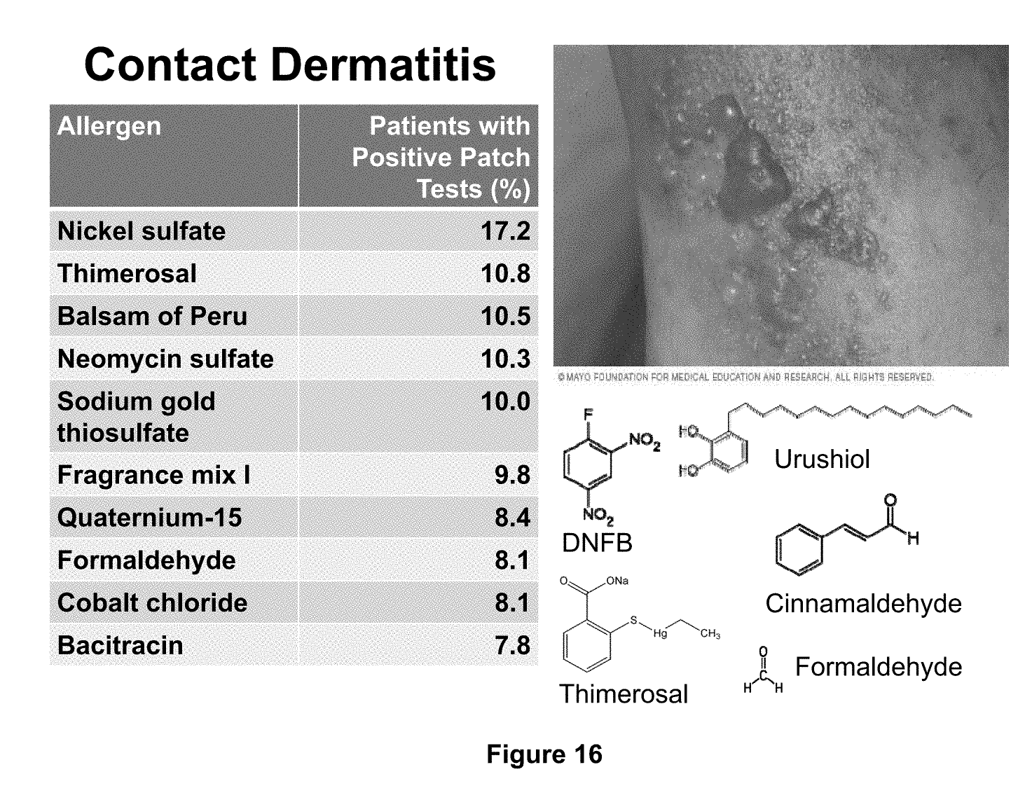

Skin is the main route to allergic sensitization and provides innate as well as adaptive immune functions to maintain homeostasis (Kaplan, D. H. et al. Nat Rev Immunol, 2012, 12:114-124). Contact sensitizers bind to self-proteins in the skin to generate antigenic-protein complexes that trigger an adaptive immune response (Divkovic, M. et al. Contact dermatitis, 2005, 53:189-200). Skin resident antigen presenting cells (APCs) polarize effector CD4⁺ and CD8⁺ T cells in the lymph nodes against the antigen following first contact (sensitization phase) and upon subsequent exposure (challenge phase) a pruritic rash and/or swelling results from the response of the antigen-specific T cells (Watanabe, H. et al. Journal of interferon & cytokine research, 2002, 22:407-412; Vocanson, M. et al. Expert review of clinical immunology, 2005, 1:75-86; Saint-Mezard, P. et al. J Invest Dermatol, 2003, 120:641-647; Honda, T. et al. J Invest Dermatol, 2013, 133:303-315; Martin, S. F. et al. Int Arch Allergy Immunol, 2004, 134:186-198). Common sensitizers include urushiol, nickel, latex rubbers, acrylics and fragrances (FIG. 16) (Cashman, M. W. et al. Dermatologic clinics, 2012, 30:87-98). Personal skin care products, jewelry and workplace exposures are the most common routes of sensitizer skin contact (Cashman, M. W. et al. Dermatologic clinics, 2012, 30:87-98). Application of topical corticosteroids is the primary treatment to minimize pruritus and skin inflammation (Usatine, R. P. & Riojas, M. American family physician, 2010, 82:249-255; Levin, C. & Maibach, H. I. Contact Dermatitis, 2000, 43:317-321; Bourke, J. et al. Br J Dermatol, 2009, 160:946-954). To decrease the risk of chronic dermatitis the sensitizer must be identified via a patch test and contact avoided (Rietschel, R. L. J Am Acad Dermatol, 1995, 33:812-815).

In vivo contact hypersensitivity (CHS) mouse models have widely been used to study the immunologic mechanisms ACD (Christensen, A. D. & Haase, C. Apmis, 2012, 120:1-27; Martin, S. F. Methods Mol Biol, 2013, 961:325-335; Allen, I. C. Methods Mol Biol, 2013, 1032:139-144;). For the sensitization phase, on day 0 the animal is exposed to a fixed concentration of a chemical hapten (e.g. 1-fluoro-2,4-dinitrobenzene (DNFB), oxazolone, phthalic anhydride). For the challenge phase, 5 days later the animal is re-exposed to the same hapten, typically on the ear (Saint-Mezard, P. et al. J Invest Dermatol, 2003, 120:641-647; Gaspari, A. A. & Katz, S. I. Current protocols in immunology, 2001, Chapter 4, Unit 4 2). This leads to secretion of cytokine mediators by skin cells, activation of skin resident APCs and recruitment of antigen-specific T cells to the application site causing inflammation (Gaspari, A. A. & Katz, S. I. Current protocols in immunology, 2001, Chapter 4, Unit 4 2; Lee, H. Y. et al. Mediators of inflammation, 2013, 916497). Keratinocytes, neutrophils and mast cells are also involved in the challenge phase (Honda, T. et al. J Invest Dermatol, 2013, 133:303-315).

Recently, the potential role of nanoparticles (NPs) in modulating skin inflammatory responses using ACD and atopic dermatitis mouse models has been investigated with differing results. For example, using a nickel allergy C3H/HeJ mouse model it was reported that topical application of CaCO₃ NPs (<500 nm) reduced dermatitis symptoms as well as the penetration of nickel ions into skin (Vemula, P. K. et al. Nat Nanotechnol, 2011, 6:291-295). This was a chelation effect on the skin surface that prevents nanoparticle penetration into the skin and had no effect on the skin immune repertoire. Similarly, using a DNFB CHS BALB/c mouse model it was reported that the application of silver NPs applied once a day for 4 days post challenge resolved inflammatory symptoms to levels attained with a macrolide immunosuppressant and caused apoptosis of immune cells (Bhol, K. C. & Schechter, P. J. The British journal of dermatology, 2005, 152:1235-1242). Others found that subcutaneous injection of TiO₂ NPs in BALB/c mice increased the sensitization potential of 2,4-dinitirochlorobenzene indicating an adjuvant effect (Hussain, S. et al. Part Fibre Toxicol, 2012, 9:15). Although the above mentioned studies and others use different models, a common protocol is to apply multiple applications of NPs extending over many days (fives, M. et al. Part Fibre Toxicol, 2014, 11:38). There have been no studies investigating the immunomodulatory effect of NPs applied as a single topical dose in either the sensitization or challenge phase using a murine CHS model.

The methods and materials are now described.

### Instruments and Software

Digital calipers manufactured by Kroeplin (#C11OT) were used to measure the mouse ears. Cryotome FE (Thermoscientific) was used to section the mouse ear tissue for histology. Zetasizer Nano (Malvern Instruments) and Nanodrop was used to quantify the size, charge, and concentration of the QD samples. Nikon E800 microscope was used to image the histology sections and RT3 camera/spot advanced software (version 4.6) was used to acquire the images.

### Confocal Imaging: Analysis of Stacks

The system was adjusted so negligible autofluorescence was observed at the QD emission peak (605 nm) in the control mouse skin. Images obtained using CLSM were processed using Image J Analysis software (NH, version 1.48). Each image (8 bit) was split into 3 channels, the red channel (QD) was retained for analysis and the pixel information was extracted using the histogram function. A high threshold for fluorescence signal between 220 and 255 on the grey scale representing QDs was set on Image J for the purpose of quantification. The pixel number was averaged to obtain relative intensity of the QDs in each individual image between the depths of 0-40 μm. A cut off depth of 40 μm for imaging was set to quantify penetration differences into viable epidermis in the different treatment groups.

### DNP Assay to Detect DNFB-Protein Adducts

Ear tissue was obtained from mice sensitized with 0.05% DNFB and challenged with 0.2% DNFB (right ear) and 0.2% DNFB+NP combination (left ear). The NPs used in this experiment included 20 nm and 400 nm SiNPs, glutathione coated QDs (GSH QDs) and multi-walled carbon nanotubes (MWCNTs). Tissue was also collected from treated mice 24 hours after the DNFB application. OxyBlot™ protein oxidation detection kit was obtained from Chemicon International (Catalog No: S7150) to quantify DNFB-protein adducts. Briefly, whole skin homogenates were separated by SDS PAGE and transferred to 0.2 micron nitrocellulose by Western blot. The blots were treated with the primary and secondary antibodies in the Oxyblot kit, and the protein band intensity was quantified by a Gel-Doc system (Biorad). The loading of each sample was controlled by the colorimetric analysis of a ponceau total protein stain.

### Quantum Dot (QD) Functionalization

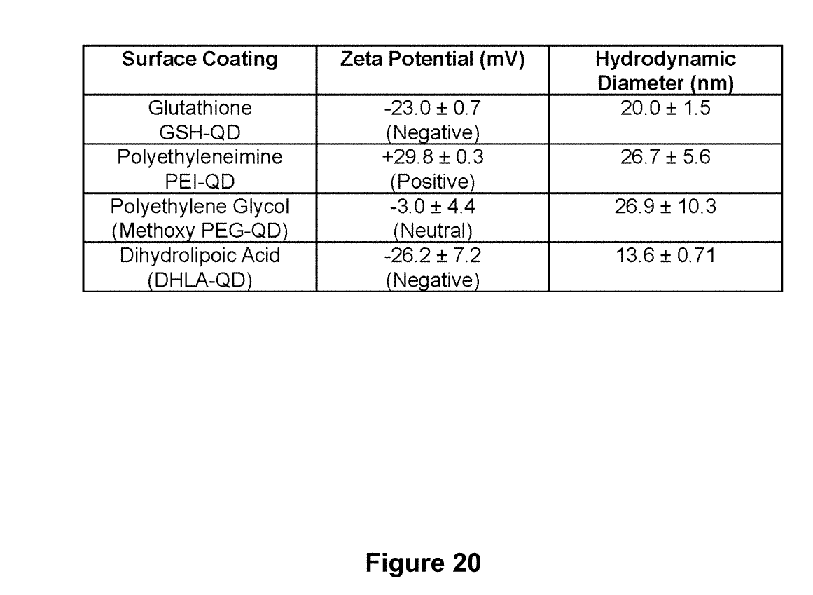

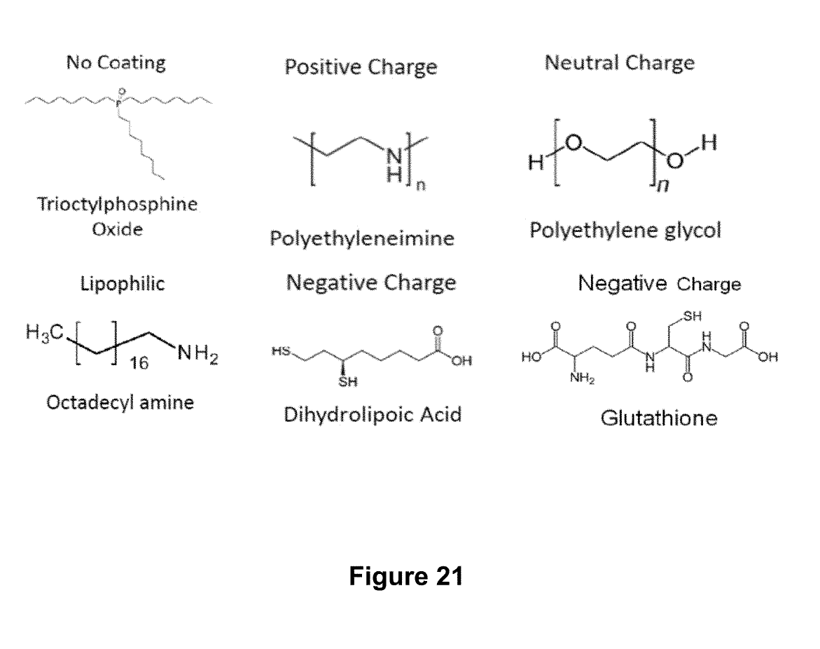

Commercially available Cadmium Selenide-Zinc Sulphide (CdSe—ZnS, 5.8 nm core diameter, 600-620 nm emission peak) core-shell nanocrystals dissolved in toluene and capped with trioctylphosphine oxide (TOPO) were modified using ligand exchange technique to render them water-soluble. QDs were coated with Glutathione (GSH, negative surface charge), polyethylenimine (PEI, positive surface charge), dihydrolipoic acid (DHLA, negative surface charge) and methoxy polyethylene glycol (Me-PEG, neutral surface charge) to alter the surface charge. The concentration of the sample was determined by measuring the UV-Vis absorbance on a Nanodrop spectrophotometer at the first exciton using Lambert-Beer's Law. The Malvern Zetasizer Nano ZS was used to determine the hydrodynamic diameter by light scattering and surface charge by zeta potential measurements made in distilled water (pH=6.7). The QD properties have been summarized in FIG. 20.

### In Vivo Nanoparticle Sensitization and Challenge Experiments

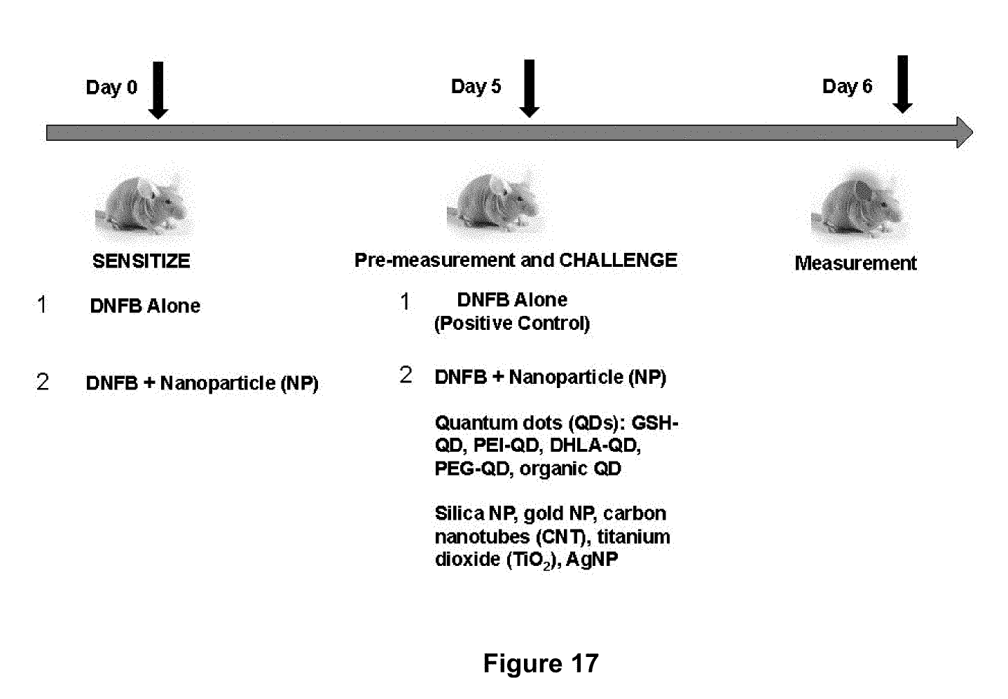

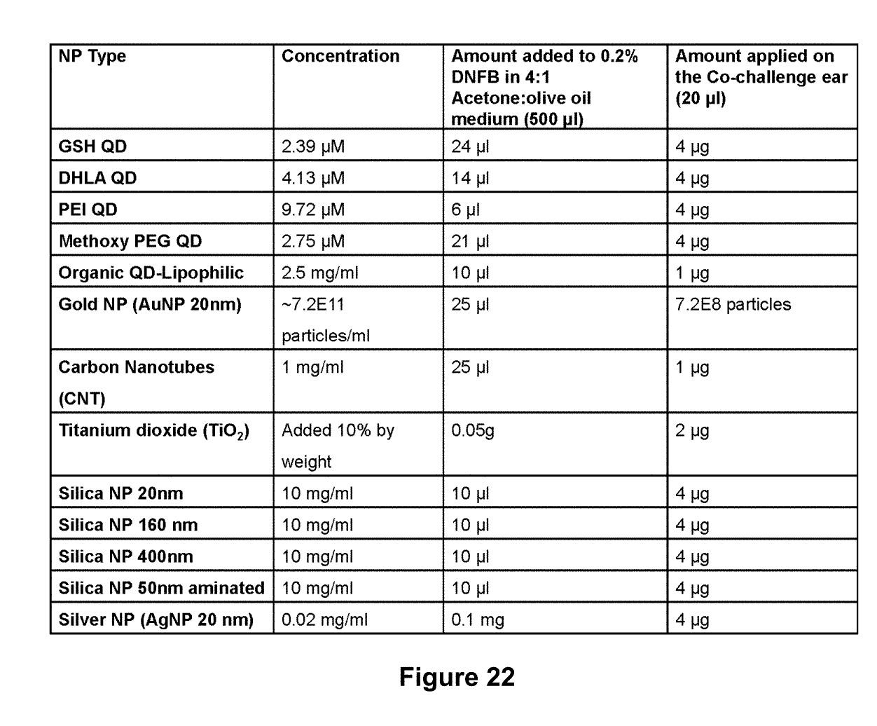

All mice used in this study are hairless C57BL/6, which contain a genetic mutation that causes alopecia to develop after the first hair follicle maturation. This phenotype is preferred for topical exposures, since the use of other breeds necessitates hair removal, which may cause a barrier defect in the epidermis and hence facilitate NP penetration. The mice do not have hair but their hair follicles are intact. Mice were either male or female with ages that range from 5-6 months old. The mice were housed in standard cages, up to four mice per cage, with access to food and water ad libitum. However, after sensitization, the mice were housed individually to prevent grooming. The schematic for CHS protocol is outlined in FIG. 17. Briefly, mice were sensitized on Day 0 using 0.05% 1-fluoro-2,4-dinitrobenzene (DNFB), a common Th1 chemical hapten, in an acetone/olive oil vehicle (4:1 acetone:olive oil volume ratio). The sensitization dose (30 μl) was pipetted on dorsal side right above the tail region. 5 days post-sensitization, both the right and left ear were premeasured using digital calipers prior to the application of the challenge dose. A challenge dose of 20 μl, 0.2% DNFB in 4:1 acetone:olive oil vehicle, was pipetted on the right ear (10 μl on each the dorsal and ventral side of the ear). The left ear was challenged using the vehicle alone (20 μl). 24 hours post-challenge, the ear swelling response was measured using calipers. The mice were euthanized via CO₂ asphyxiation and the ear tissue was collected and stored at −80° C. for staining. In the co-sensitization study, GSH QDs and PEI QDs were mixed with 0.05% DNFB in acetone/olive oil vehicle in a fixed concentration and applied on the dorsal side on day 0. In the co-challenge study, the different NP types were mixed with 0.2% DNFB and applied on the left ear, the right ear was challenged using 0.2% DNFB alone (FIG. 22). In both the co-sensitization and co-challenge study, the DNFB was in molar excess of the NP concentration. For the 2-deoxyurushiol studies, 100 ul of 15% 2-deoxyurushiol in an acetone vehicle was pipetted onto the dorsal side of the mouse just above the tail for the sensitization. After 5 days, 20 ul of the 15% 2-deoxyurushiol solution was pipetted onto the ear (10 ul on both the dorsal and ventral sides). One ear was treated with 2-deoxyurushiol alone and the other received both 2-deoxyurushiol and 20 nm silica nanoparticles (20 μg total dose).

### Quantification of the Ear Swelling Response

Both right and left ear thickness was measured using digital calipers on Day 5 before the application of the challenge dose and recorded as the pre-challenge ear thickness. Twenty four hours after challenge, the swelling response was measured and recorded as the post-challenge ear thickness. Data are expressed as follows: change in ear thickness=(post-challenge ear thickness)−(pre-challenge ear thickness). To examine the ear swelling via a secondary qualitative method, 5 μm frozen sections of the ears were cut using a Thermo Scientific Cryotome FE. These sections were placed on glass slides and stained with hematoxylin and eosin dye using standard procedures. General tissue histology and cell infiltrates were observed using a Nikon Eclipse E800 bright field microscope.

### Ex Vivo Mouse Skin QD Exposure and Quantification Using Confocal Microscopy

In order to quantify QD penetration through mouse skin, all QD types were topically applied on mouse skin (4 cm² area) in the acetone/olive vehicle for a duration of 24 hours at room temperature in an ex vivo set up. Skin samples were placed in a petri dish on gauze soaked in culture media to keep the skin hydrated during the topical exposure. After the 24 hour exposure, the residual vehicle was wiped off the stratum corneum and the skin samples were imaged using confocal laser scanning microscopy (CLSM) from 0-40 μm into the viable epidermis. The system was adjusted so negligible autofluorescence was observed at the QD emission peak (605 nm) in the control mouse skin. The stacks collected using CLSM were quantified for QD presence using the histogram function on Image J analysis software. Each image (8 bit) was split into 3 channels, the red channel (QDs) was retained for analysis and the pixel information was extracted using the histogram function. A high threshold for fluorescence signal between 220-255 on the grey scale representing QDs was set on Image J for the purpose of quantification. The pixel number was averaged to obtain relative intensity of the QDs in each individual image between the depths of 0-40 μm. A cut off depth of 40 μm for imaging was set to quantify penetration differences into viable epidermis in the different treatment groups.

### Statistical Analysis

Two-tailed Student's t-test, unpaired with unequal variances, was used to compare penetration differences between different QD applications in the ex vivo penetration study (N=5). 2-tailed Student's t-test, paired with unequal variances, was used to compare the ear swelling measurements. Data are represented as change in swelling response compared to the premeasurement value before the challenge (baseline thickness of the ear). P<0.05 was considered to be significant. Error bars represent standard error of mean (SEM). The number of mice used in each experiment has been mentioned under individual plots.

### Results and Discussion

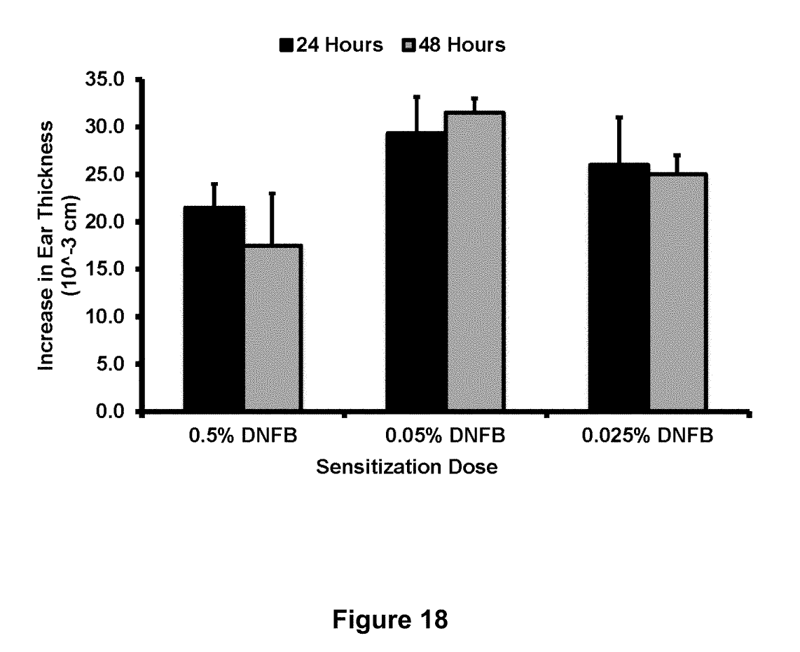

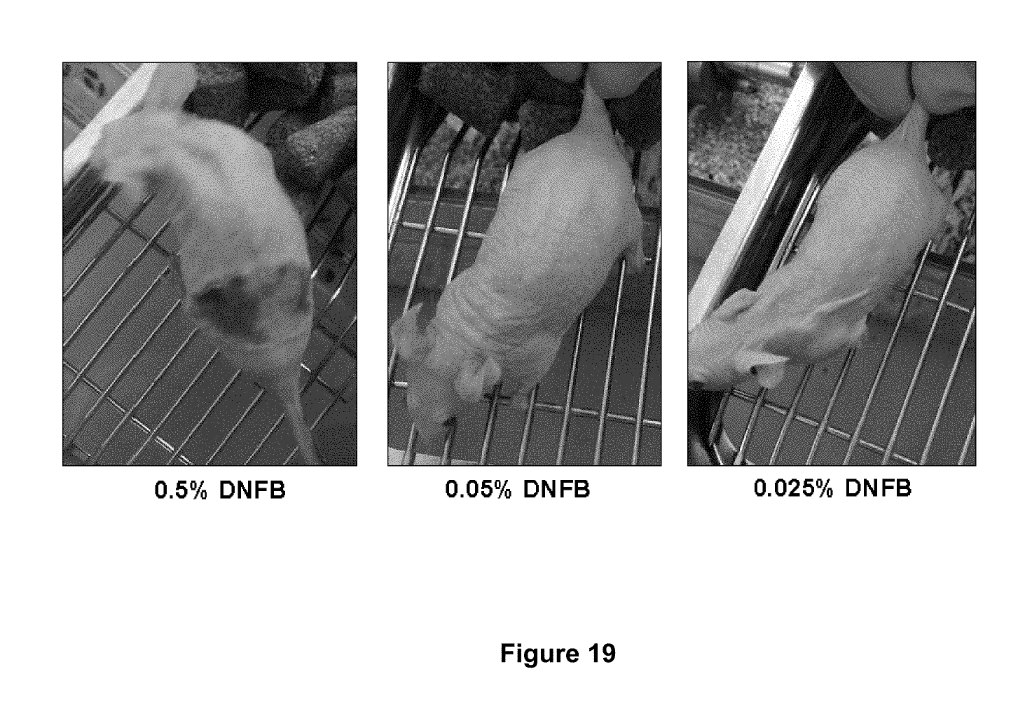

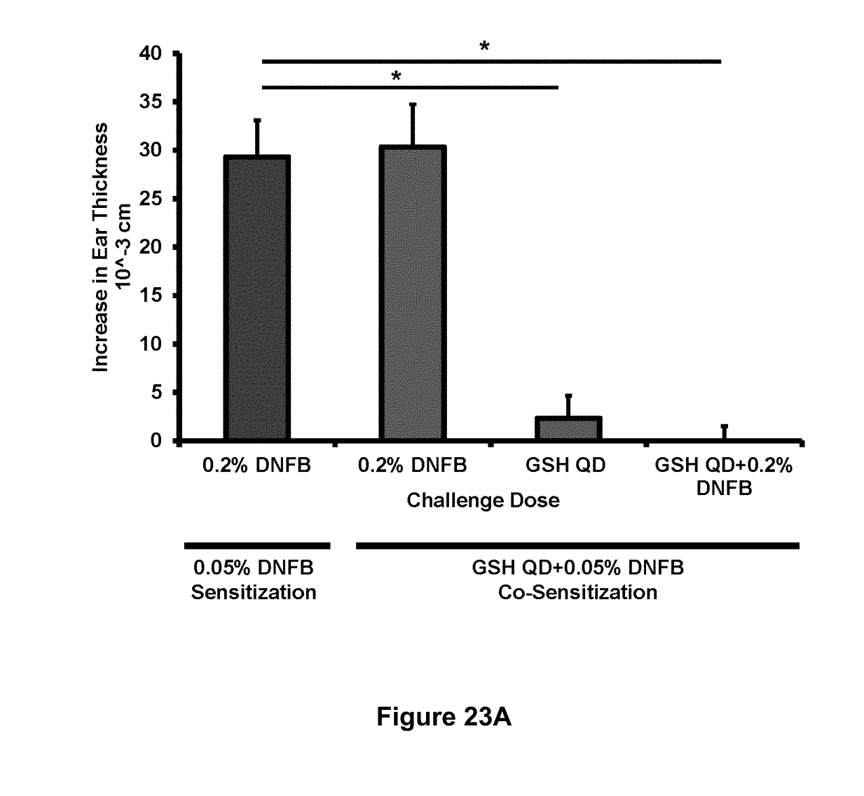

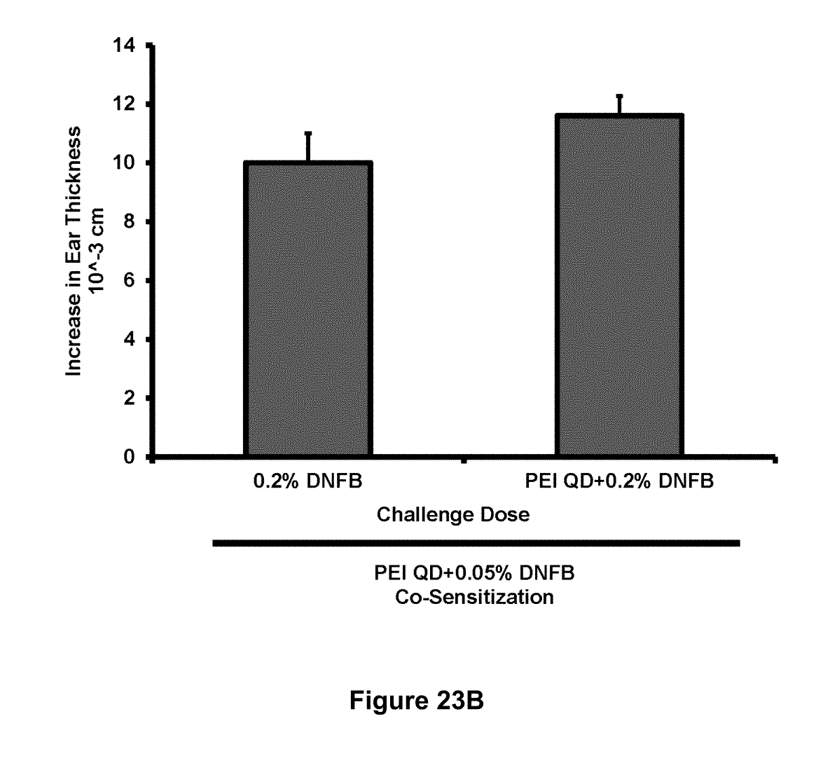

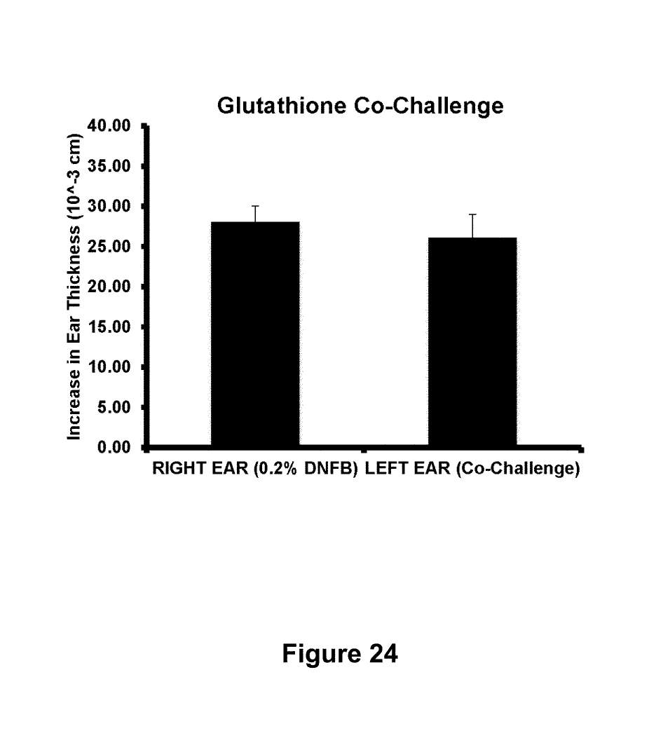

This study examined the ability of topically applied NPs to modulate the CHS response to DNFB and 2-deoxy urushiol. In all experiments, sensitization was done with 0.05% DNFB and challenge with 0.2% DNFB in 4:1 acetone/olive oil vehicle alone or in combination with NPs (FIG. 18 and FIG. 19). First, the impact of negatively charged glutathionecoated quantum dots (GSH-QDs) on the sensitization phase was tested (FIG. 17, FIG. 20, FIG. 23A, FIG. 23B). GSH-QDs were mixed with DNFB and applied to the backs of the mice. The mice were challenged on the ear with DNFB alone, QDs alone or co-challenged with DNFB+QDs. In the GSH-QD/DNFB sensitized group, an ear swelling response was not observed when the mice were challenged with GSH-QDs alone, indicating that even in the presence of the powerful DNFB sensitizer the mice could not be sensitized to the QDs (FIG. 23A). Upon challenge with 0.2% DNFB, the expected magnitude ear swelling response was measured, indicating that the mice were sensitized to the hapten and that the presence of GSH-QDs during sensitization did not hinder or alter the antigenic epitope formation (FIG. 23A). However, when co-challenged with DNFB+QDs the swelling response was significantly abrogated, indicating that the GSH-QDs exerted an inhibitory effect on ear swelling despite the fact that the DNFB was applied in an estimated >105 molar excess to QDs (FIG. 23A). Co-challenge with glutathione alone had no immunosuppressive effect (FIG. 24).

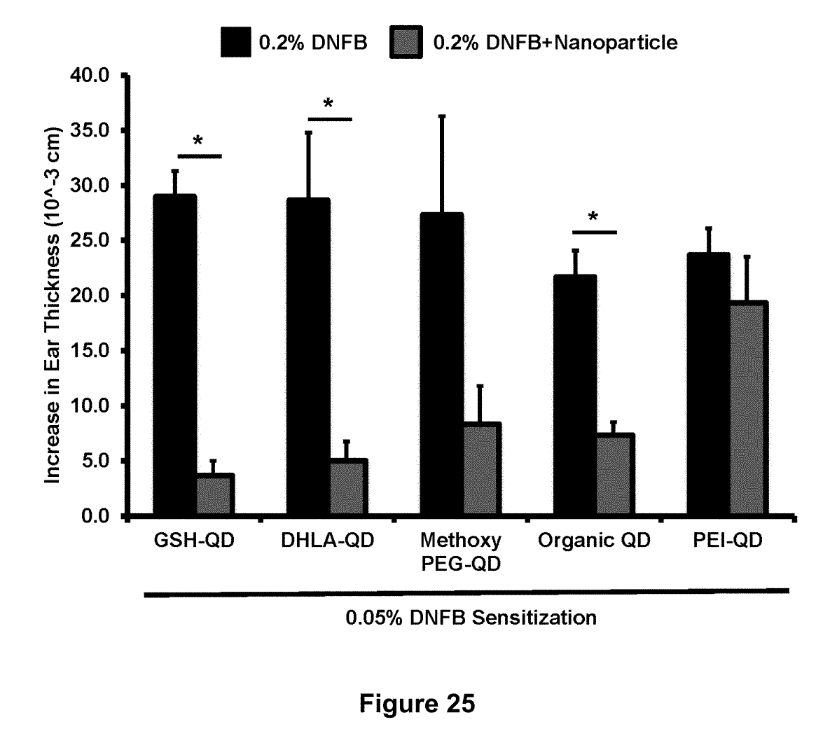

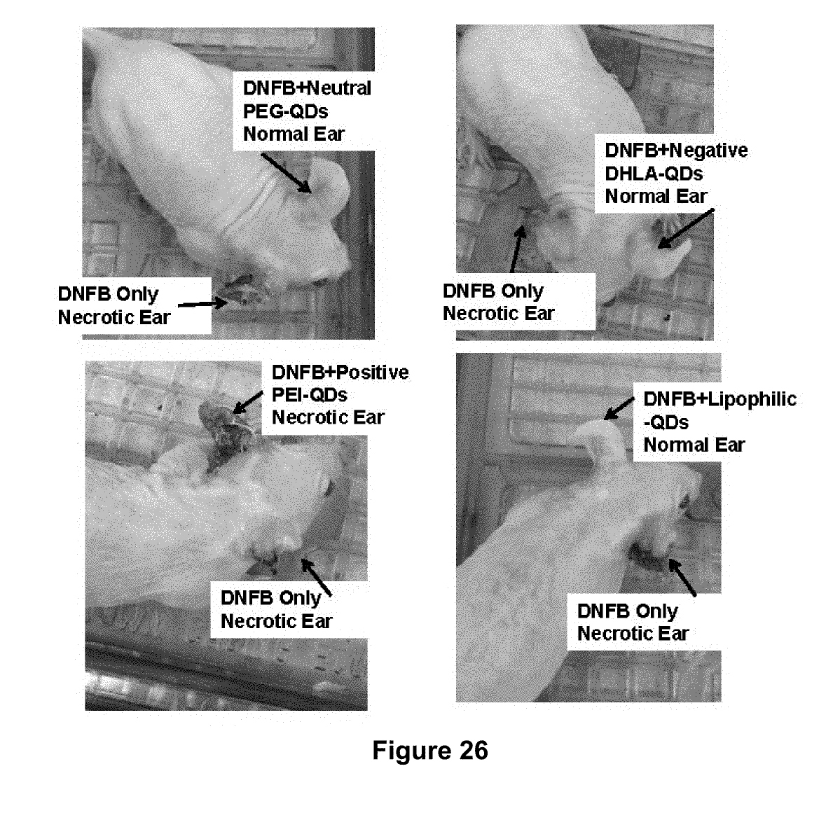

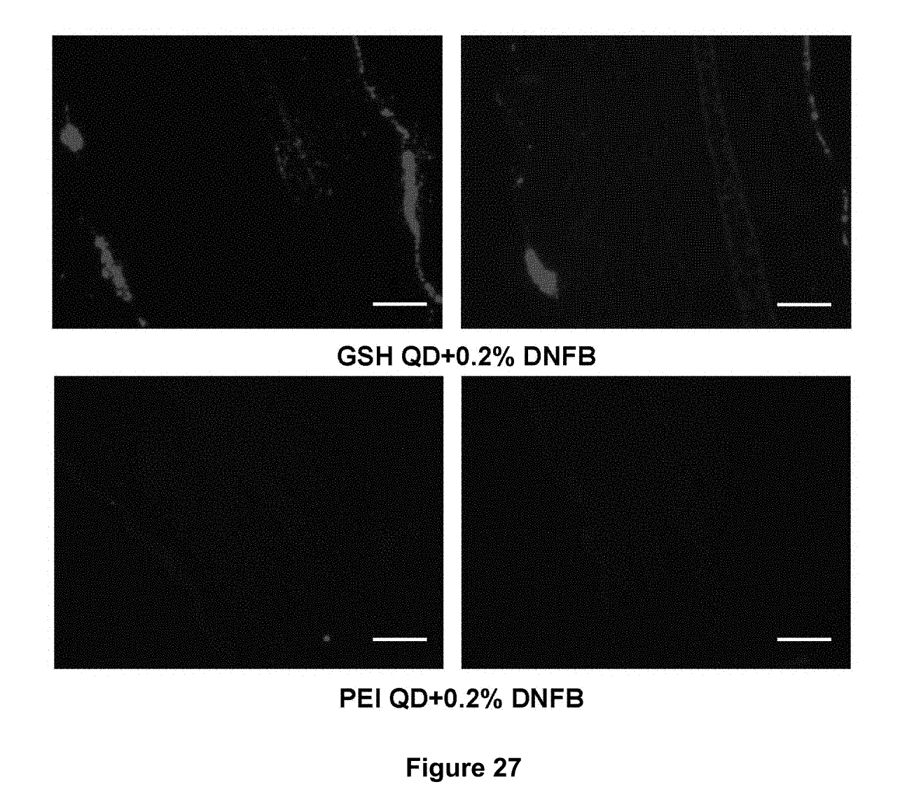

Ear swelling responses were also measured on mice that were co-sensitized with DNFB+PEI-QDs and challenged with DNFB alone or co-challenged with DNFB+PEI-QDs. In contrast to the GSH-QDs, results show that the positively charged PEI-QDs did not suppress ear-swelling response (FIG. 23B). This result demonstrates that the immunosuppressive mechanism may be dependent on NP charge, which may affect either the bioavailability of DNFB in skin or the ability of the NPs to alter the local immune response in the skin during the challenge phase; again despite the fact that the DNFB was applied in molar excess compared to QDs. The immunosuppressive potential of QDs was then tested with additional surface coatings and charge in the challenge phase alone (FIG. 20, FIG. 22). Here, the mice were sensitized with DNFB only and co-challenged with the DNFB+QDs. Results (FIG. 25, FIG. 26, FIG. 27) show that DHLA-QDs (negative charge), methoxy PEG-QDs (neutral) and organic QDs (octadecylamine, ODA) all inhibited the ear swelling response as did GSH-QD (negative charge).

Consistent with the co-sensitization results (FIG. 23B), the PEI-QDs (positive charge) did not suppress swelling (FIG. 25). The mice were observed for 1 month after challenge. The ears that were affected by the DNFB hapten were necrotic (FIG. 26). However, the co-challenge ears (left) in the case of DHLA-QD, PEG-QD, and lipophilic QDs appeared normal (FIG. 26). Whereas, the ear co-challenged with DNFB-PEI-QD was also necrotic since the presence of PEI-QDs in the challenge phase did not inhibit the ear swelling response.

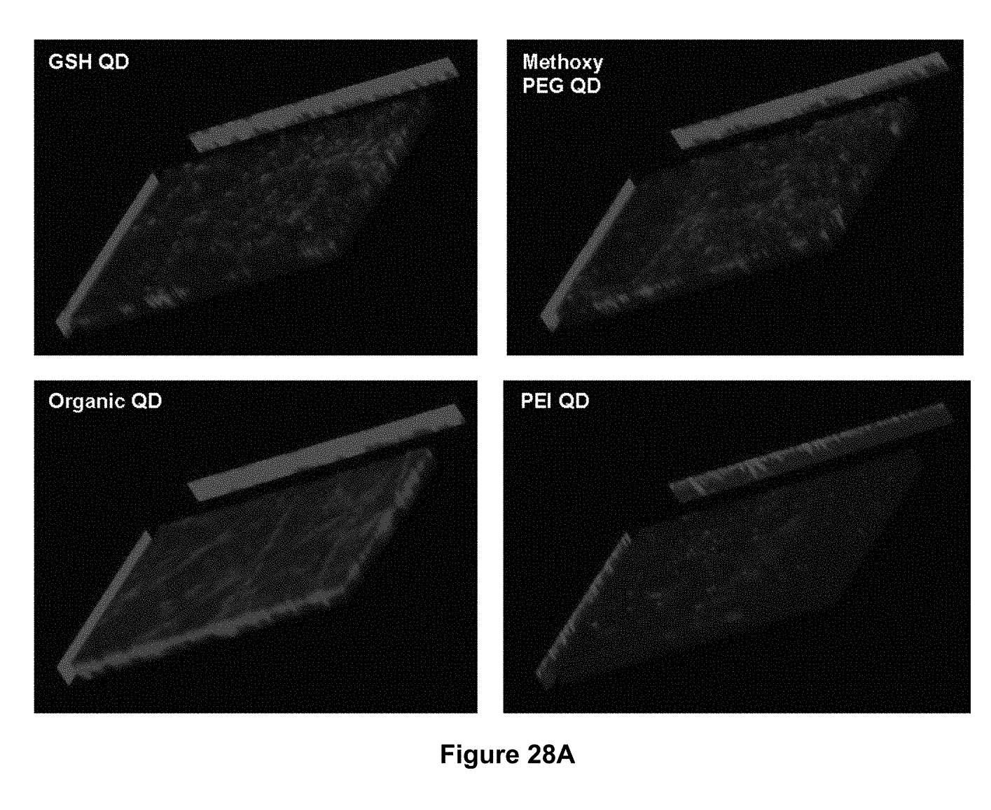

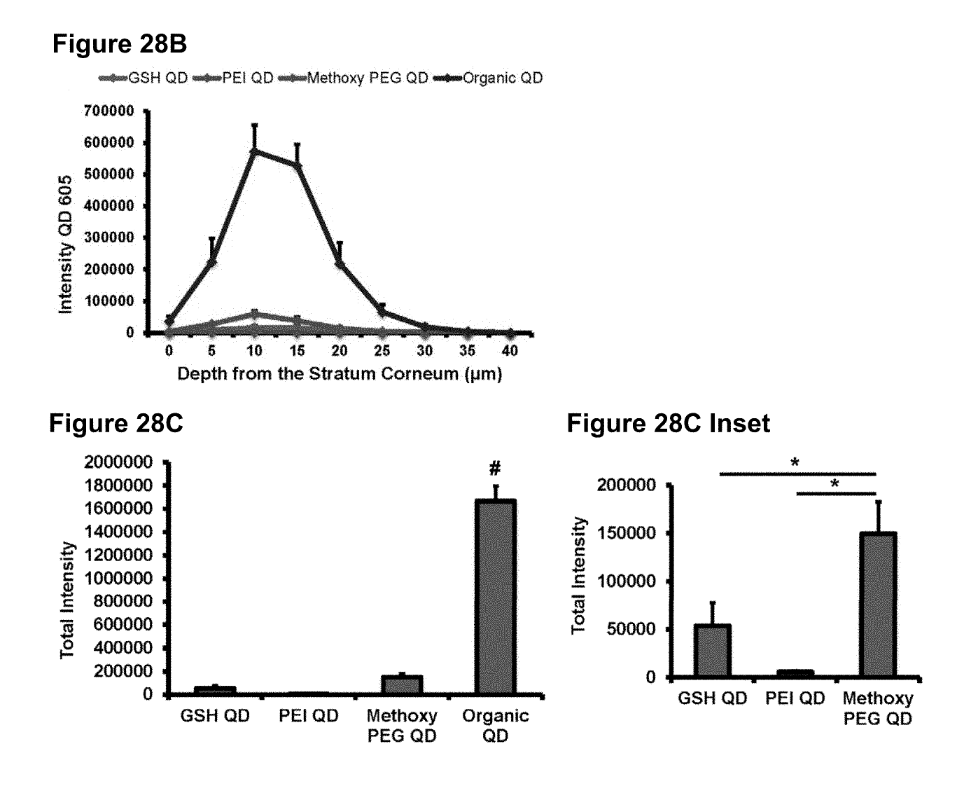

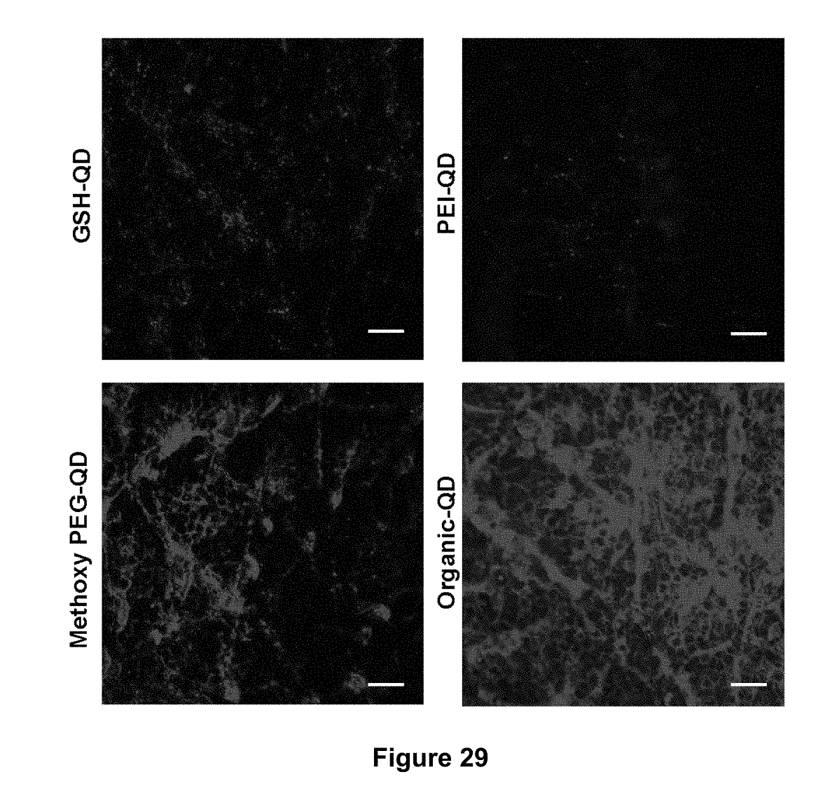

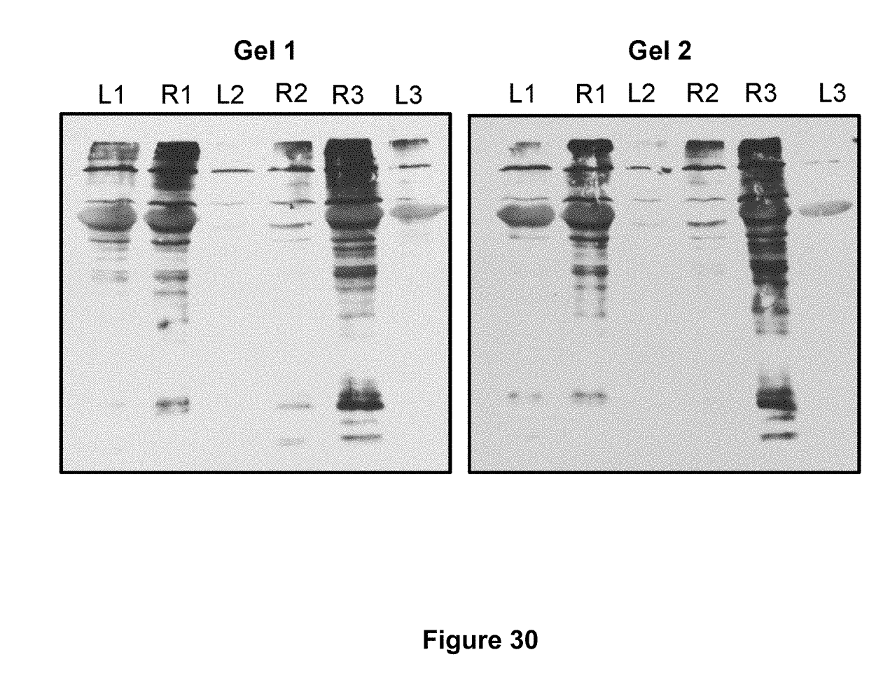

To investigate the potential for QDs to interact with skin cells, QD penetration studies were performed on ex vivo mouse skin using scanning confocal microscopy as previously described (Jatana, S. et al. Nanoparticle Penetration through ex vivo C57BL/6 Hairless Mouse and Human Skin: A Comparison Study. Under review, 2015). Results showed that the PEI-QDs mainly concentrated in the outermost stratum corneum skin layer but follicular accumulation is evident (FIG. 28A through FIG. 28C, FIG. 29). Quantitative analysis of the image stacks (0-40 μm) show that the organic QDs appear to penetrate to a greater extent compared to all other treatment groups especially when examining the magnitude of the QD fluorescence signal below the stratum corneum (FIG. 28B, FIG. 28C). PEI-QDs penetrate the least through the stratum corneum and were observed to be concentrated in the hair follicle areas. Cryosections of the ear tissue were also examined for QD presence and a greater presence was observed in the tissue treated with GSH-QDs compared to PEI-QDs. GSH-QDs were present in the stratum corneum, epidermis, dermis, as well as the cartilage area (FIG. 27). This data demonstrates that penetration of positively charged QDs is hindered possibly through agglomeration and/or electrostatic attraction to the negatively charge skin surface which thereby limits their potential to interact and modulate skin cell responses.

Western blot analysis was used to examine whether the negatively charged QDs alter the bioavailability of the DNFB to form antigenic adducts in skin. Quantification of western blot data 24 hr post-challenge does show a trend towards decreasing DNFB-protein adduct levels in the DNFB+GSH-QD co-challenged ear compared to DNFB alone treated ear. However, a significant presence of adducts was evident and moreover, the weaker staining could reflect an enhanced clearance or altered epitope recognition by the antibody used in this assay. The DNFB protein adduct formation quantification and analysis is discussed in Example 3.

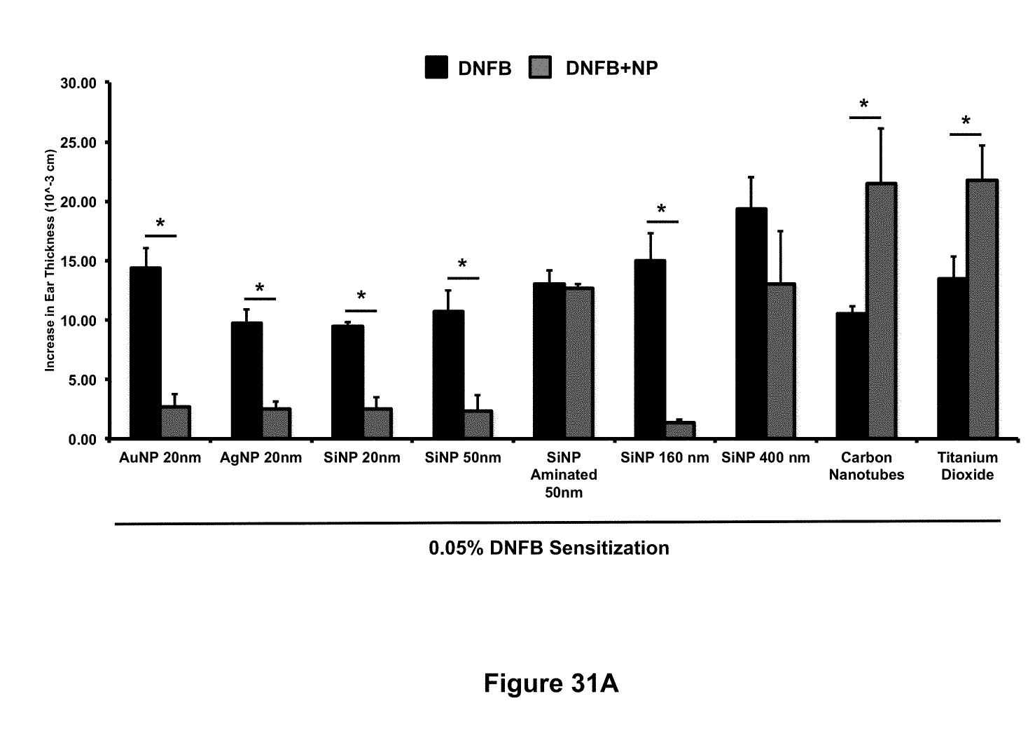

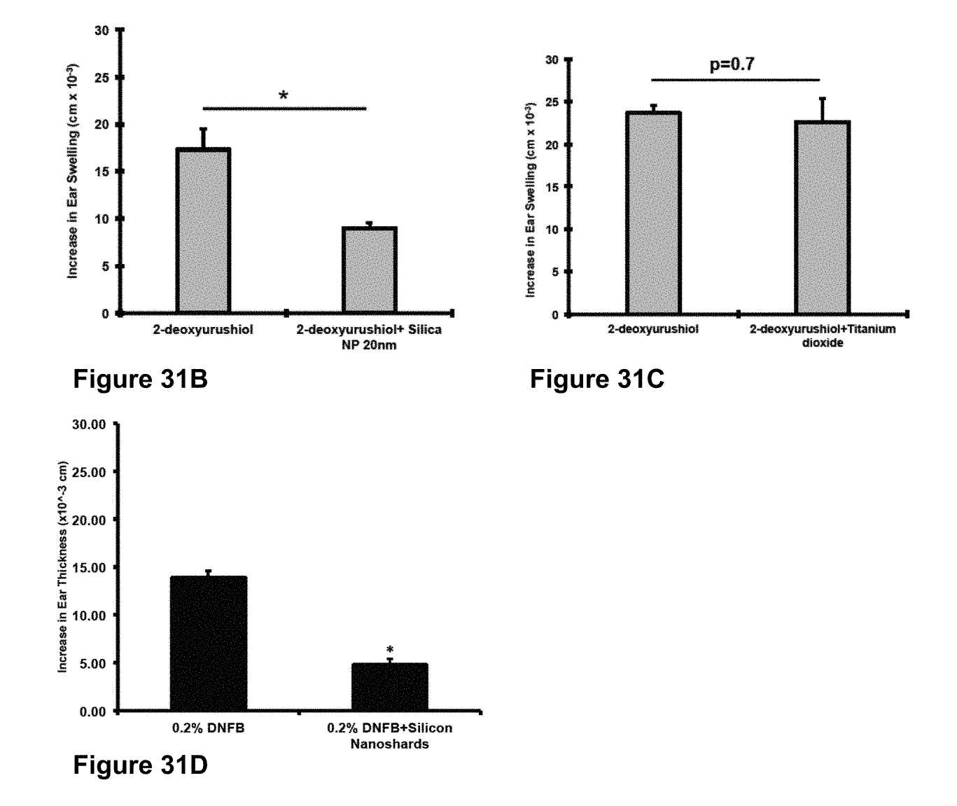

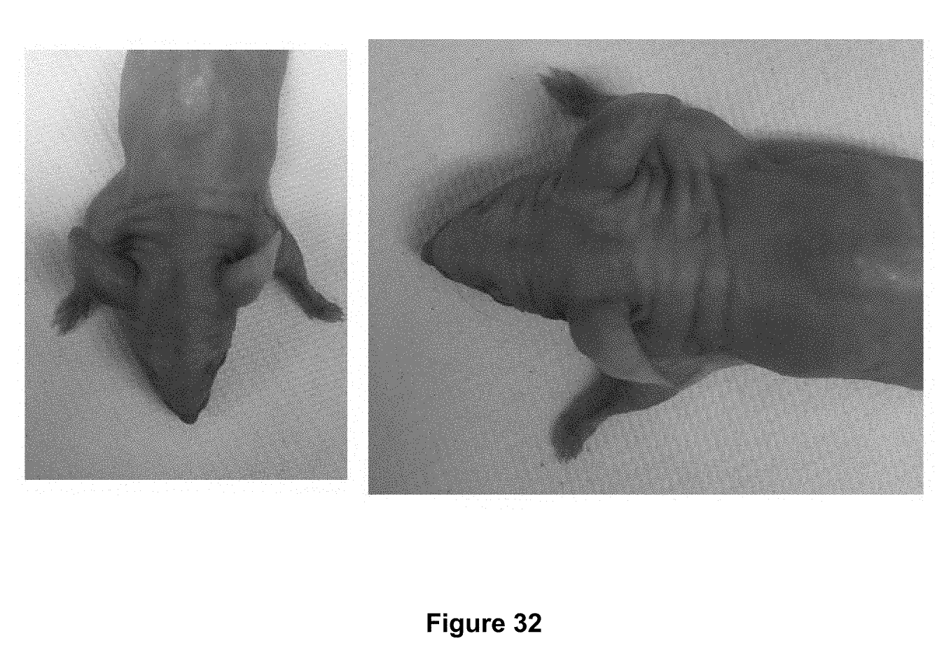

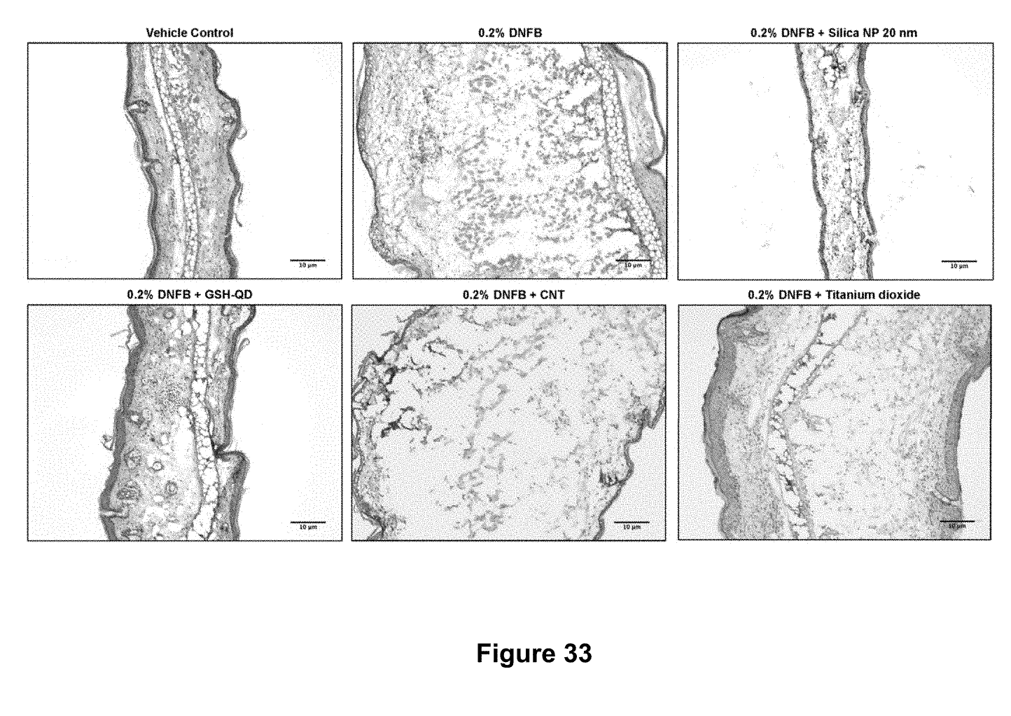

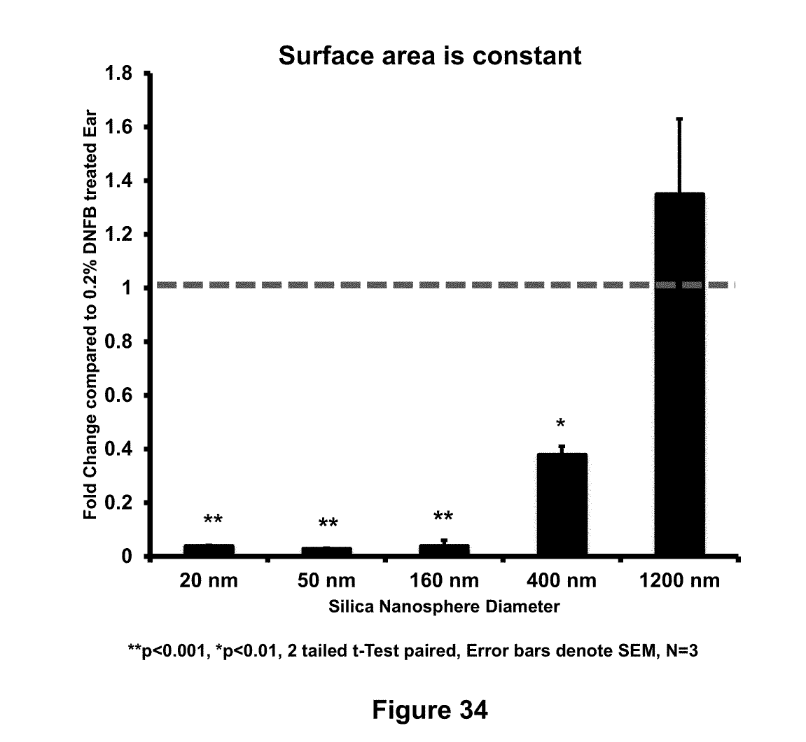

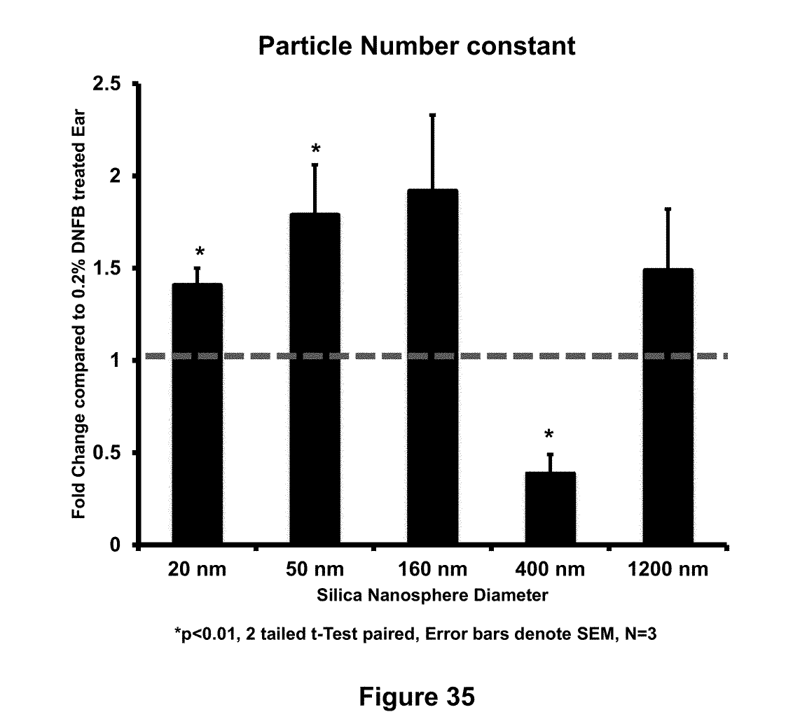

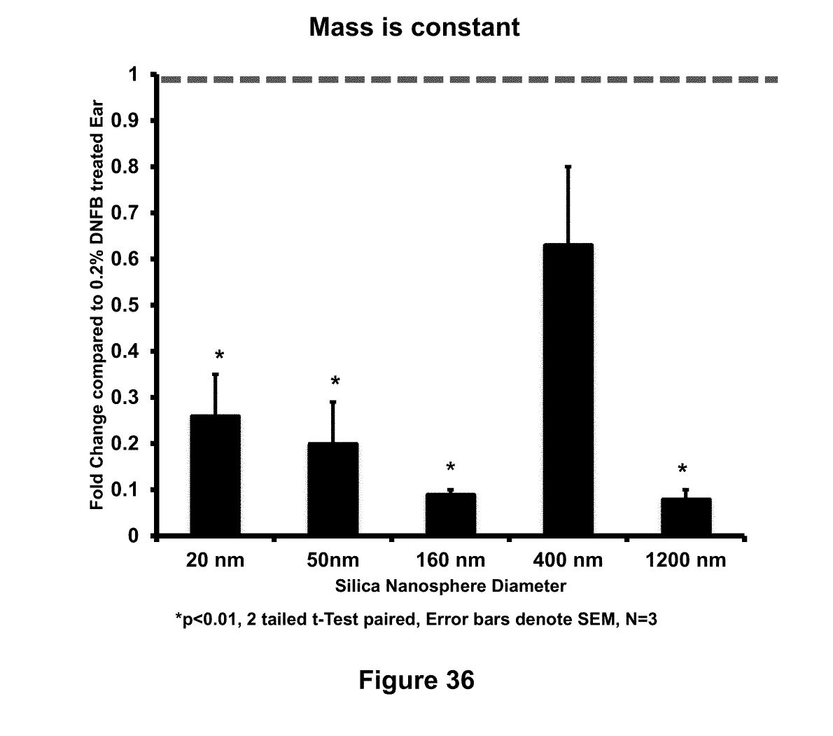

To further test whether this immunosuppressive response is specific to small core diameter (˜6 nm) QDs, other NP types were examined, including citrated gold NPs (20 nm), hydroxylated silica NPs (20 nm, 50 nm, 160 nm) and citrated silver NPs (20 nm), which are all negatively charged (FIG. 22). Results indicate that all these NPs suppress the ear swelling response compared to the DNFB treated ear alone (p<0.05) (FIG. 31A). Gross examination of the mouse ear 24 hours after challenge shows swelling observed in the DNFB treated ear (right) and no inflammation in the co-challenged DNFB+Silica NP (20 nm) ear (left) (FIG. 32). Qualitative analysis of ear sections (5 μm thick) stained with H&E revealed fewer cell infiltrates in the immunosuppressed groups (GSH-QD, Silica NP 20 nm) compared to 0.2% DNFB treated ear (FIG. 33). Interestingly, positively charged aminated silica NPs (50 nm) and negatively charged silica NPs (400 nm) did not suppress the swelling response significantly, demonstrating that both charge and size matter. Furthermore, carbon nanotubes (CNT) and titanium dioxide (TiO2, Evonik P25, 20 nm) both exacerbate the swelling response (FIG. 31A). The exacerbation of the swelling response in the presence of CNT and TiO₂ swelling was confirmed by H&E stained ear sections (FIG. 33). Cellular infiltrates appeared to be higher in the CNT co-challenge group and tissue necrosis was also observed in the stratum corneum (FIG. 33). The TiO₂ response is not unexpected as it is widely known that TiO₂ NPs agglomerate on the skin surface and do not penetrate much beyond layers in the stratum corneum (Monteiro-Riviere, N. A. et al. Toxicological sciences, 2011, 123:264-280). Moreover, a recent study reported that topical application of nano-TiO₂ particles to mouse ears induced an irritation swelling response in a dose dependent manner (Auttachoat, W. et al. Journal of immunotoxicology, 2014, 11:273-282). While very little is understood about CNT skin penetration, in vitro studies have shown that CNTs can induce proinflammatory cytokine responses in skin cells (Monteiro-Riviere, N. A. et al. Toxicology letters, 2005, 155:377-384). These findings collectively indicate that NP composition, ligand coating, surface charge and primary particle size and/or agglomeration state are important factors for suppressing the inflammatory response.

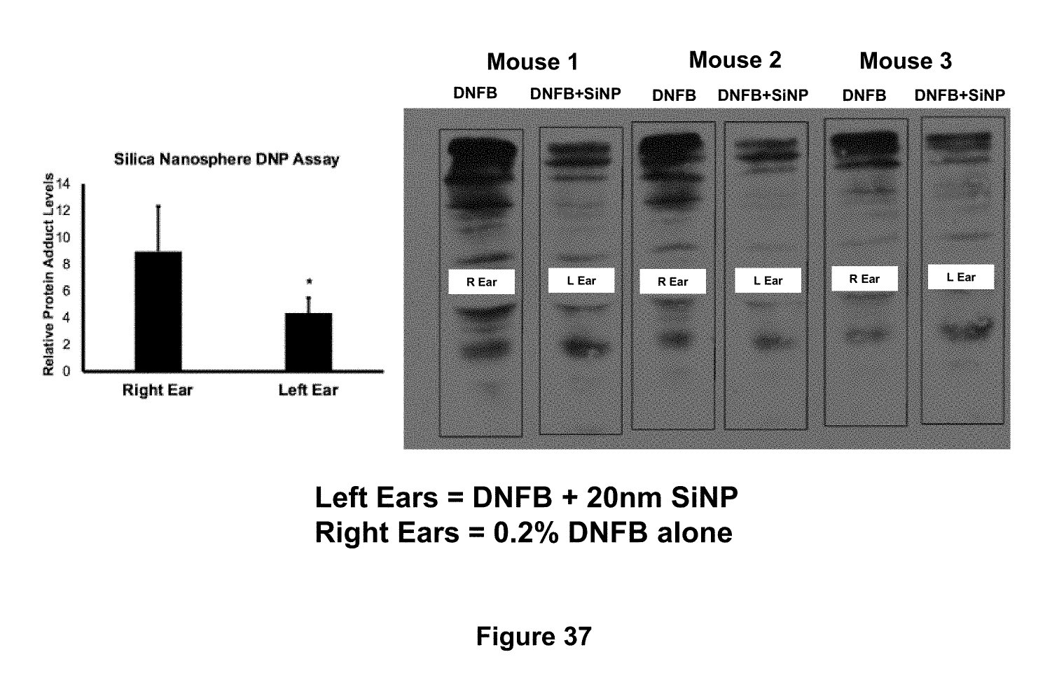

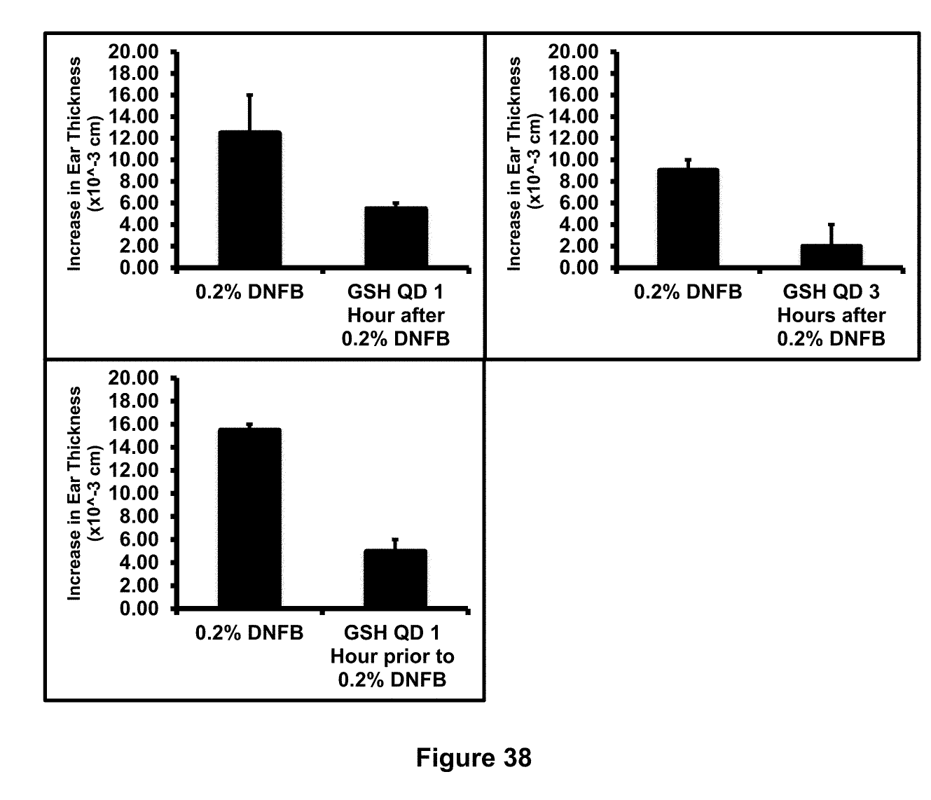

Mice were sensitized using 2-deoxyurushiol, a chemical analogue of urushiol, to examine whether these observations are specific to DNFB. Results show that the ear challenged with 2-deoxyurushiol alone exhibited the expected swelling response, however, the ear cochallenged with 2-deoxyurushiol+silica NP 20 nm had significantly lower inflammation (FIG. 31B). However, when the mice were challenged with 2-deoxyurushiol and titanium dioxide, inflammation was observed in the co-challenged ear (FIG. 31C). The urushiol result, the fact that the sensitizer in the challenge phase is applied to skin in a molar excess (˜105) to the NPs, the observed size and charge dependence, and the preliminary result to quantify hapten-protein adduct formation in skin all demonstrate that the NP immunosuppressive effect is not merely a bioavailability artifact. Fewer DNFB-protein adducts, cell infiltrates and degranulated mast cells were observed in the Silica NP (20 nm) co-challenged ear compared to the control ear (FIG. 37). Silica NPs (20 nm) inhibited the ear swelling response when the ear was co-challenged with 2-deoxy urushiol compared to control. These findings demonstrate that an NP based therapeutic is effective for treating/preventing skin inflammation. The invention also encompasses treating/preventing inflammation caused by other Th1 and Th2 sensitizing agents. The findings reported here demonstrate that some NP types have an astonishing intrinsic ability to suppress inflammatory responses when topically applied to skin in the challenge phase. This is interesting from the perspective of topical therapeutic development because most individuals are already sensitized to an allergen. This finding represents an opportunity to develop topical NP based therapeutics for treating/preventing many common inflammatory skin conditions including poison ivy, nickel allergy and contact dermatitis.

### Example 3

### Mechanistic Understanding of Immune Suppression by Nanoparticles in the Challenge Phase of the Contact Hypersensitivity Model

Several immunological mechanisms play a key role in both the sensitization and elicitation phase of the contact hypersensitivity response (CHS) and over the past decade researchers have established the roles of both immune cells and cytokines in these phases (Honda, T.; Egawa, G.; Grabbe, S.; Kabashima, K. Update of immune events in the murine contact hypersensitivity model: Toward the understanding of allergic contact dermatitis. J Invest Dermatol 2013, 133, 303-315). The following study examines possible mechanisms through which the NPs applied in the challenge phase along with an allergen (1-fluoro-2,4-dinitrobenzene, DNFB) may modulate the immune response. Low molecular weight allergens (<500 daltons) interact with skin proteins to form haptens that elicit adaptive immune responses in the Type IV hypersensitivity model (Lepoittevin, J. P. Metabolism versus chemical transformation or pro-versus prehaptens? Contact Dermatitis 2006, 54, 73-74; Kaplan, D. H.; Igyarto, B. Z.; Gaspari, A. A. Early immune events in the induction of allergic contact dermatitis. Nat Rev Immunol 2012, 12, 114-124).

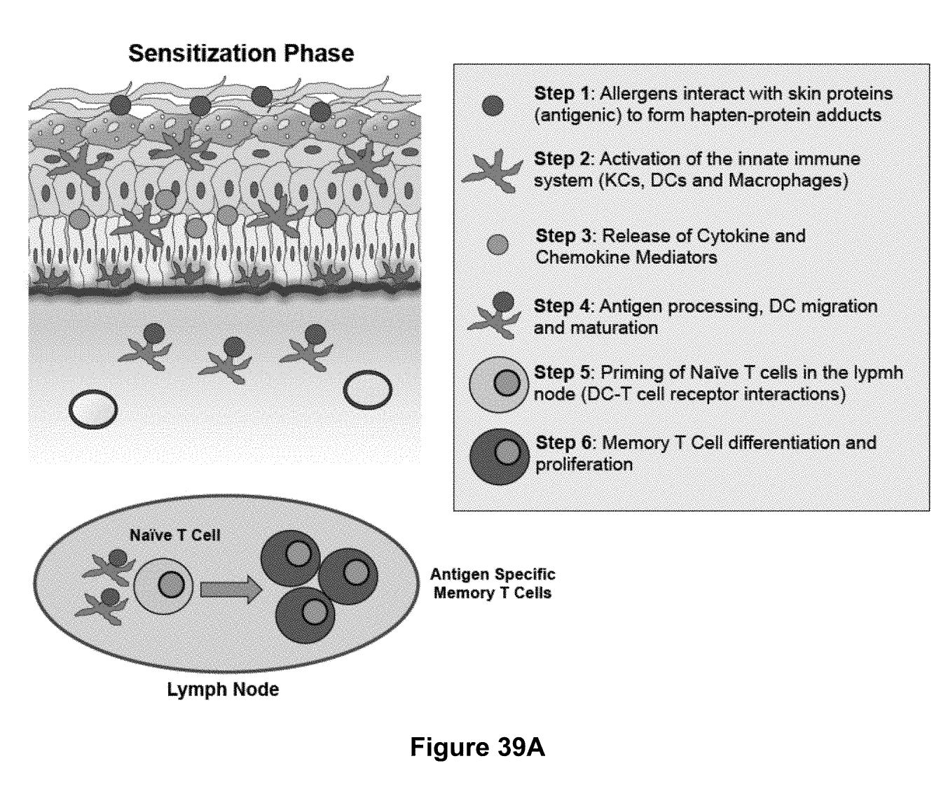

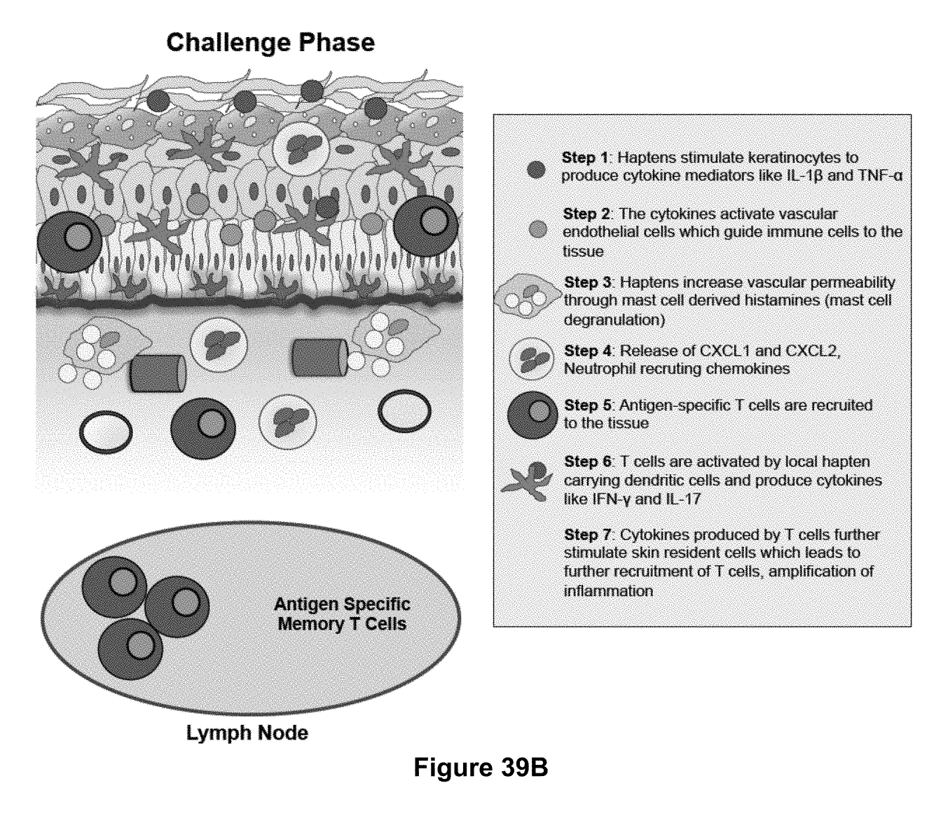

The events occurring in the sensitization and challenge phases are quite distinct and have been described in FIG. 39A and FIG. 39B. Sensitization Phase (FIG. 39A): Keratinocytes constitute a major portion of the epidermis and upon contact with the hapten produce various chemical mediators like IL-1α (IL-Interleukin), IL-8, TNFα (TNF—Tumor Necrosis Factor) and prostaglandin E2 (PGE2) (Cumberbatch, M.; Kimber, I. Tumour necrosis factor-alpha is required for accumulation of dendritic cells in draining lymph nodes and for optimal contact sensitization. Immunology 1995, 84, 31-35; Cumberbatch, M.; Dearman, R. J.; Kimber, I. Langerhans cells require signals from both tumour necrosis factor-alpha and interleukin-1 beta for migration. Immunology 1997, 92, 388-395; Honda, T.; Matsuoka, T.; Ueta, M.; Kabashima, K.; Miyachi, Y.; Narumiya, S. Prostaglandin e(2)-ep(3) signaling suppresses skin inflammation in murine contact hypersensitivity. J Allergy Clin Immunol 2009, 124, 809-818 e802). The change in the cytokine milieu upon allergen application leads to the activation of the innate immune system, primarily the skin dendritic cells (DCs). Langerhans cells (LCs) are the antigen presenting cells (APC) that abundantly exist in the skin epidermis and have been shown to play a key role in the sensitization process (Honda, T.; Egawa, G.; Grabbe, S.; Kabashima, K. Update of immune events in the murine contact hypersensitivity model: Toward the understanding of allergic contact dermatitis. J Invest Dermatol 2013, 133, 303-315). However, other studies using LC depletion systems have also emphasized the importance of dermal dendritic cells present in the dermis during this process (Bursch, L. S.; Wang, L.; Igyarto, B.; Kissenpfennig, A.; Malissen, B.; Kaplan, D. H.; Hogquist, K. A. Identification of a novel population of langerin+dendritic cells. J Exp Med 2007, 204, 3147-3156; Kissenpfennig, A.; Henri, S.; Dubois, B.; Laplace-Builhe, C.; Perrin, P.; Romani, N.; Tripp, C. H.; Douillard, P.; Leserman, L.; Kaiserlian, D., et al. Dynamics and function of langerhans cells in vivo: Dermal dendritic cells colonize lymph node areas distinct from slower migrating langerhans cells. Immunity 2005, 22, 643-654; Wang, L.; Bursch, L. S.; Kissenpfennig, A.; Malissen, B.; Jameson, S. C.; Hogquist, K. A. Langerin expressing cells promote skin immune responses under defined conditions. J Immunol 2008, 180, 4722-4727). At the cellular level, the haptens act directly or indirectly on the membrane associated Toll-like receptors (TLRs) on DCs through degraded hyaluronic acid (HA)-TLR2/4 signaling (Honda, T.; Egawa, G.; Grabbe, S.; Kabashima, K. Update of immune events in the murine contact hypersensitivity model: Toward the understanding of allergic contact dermatitis. J Invest Dermatol 2013, 133, 303-315; Martin, S. F.; Dudda, J. C.; Bachtanian, E.; Lembo, A.; Liller, S.; Durr, C.; Heimesaat, M. M.; Bereswill, S.; Fejer, G.; Vassileva, R., et al. Toll-like receptor and il-12 signaling control susceptibility to contact hypersensitivity. J Exp Med 2008, 205, 2151-2162; Martin, S. F.; Esser, P. R.; Weber, F. C.; Jakob, T.; Freudenberg, M. A.; Schmidt, M.; Goebeler, M. Mechanisms of chemical-induced innate immunity in allergic contact dermatitis. Allergy 2011, 66, 1152-1163). The hapten-protein combination is processed by skin APCs via the major histocompatibility complex II (MHCII) pathway because of the extrinsic nature of the antigen-protein complex (Kalish, R. S.; Wood, J. A.; LaPorte, A. Processing of urushiol (poison ivy) hapten by both endogenous and exogenous pathways for presentation to t cells in vitro. J Clin Invest 1994, 93, 2039-2047). The DCs mature and migrate to the draining lymph nodes to prime naïve T cells against the allergen, leading to the production of antigen specific memory effector T cells. In humans, the sensitization phase may take weeks to several months of recurrent exposures to the sensitizing chemical/allergen before these memory T cells are generated (Kaplan, D. H.; Igyarto, B. Z.; Gaspari, A. A. Early immune events in the induction of allergic contact dermatitis. Nat Rev Immunol 2012, 12, 114-124). In a mouse model of contact hypersensitivity, single or repeated exposures are sufficient to generate an immune response over a period of 5-7 days.

Elicitation/Challenge Phase (FIG. 39B): The challenge phase has two components to it, the first being the antigen-nonspecific inflammation and second the antigen-specific inflammation involving the memory T cell primed during the sensitization phase (Honda, T.; Egawa, G.; Grabbe, S.; Kabashima, K. Update of immune events in the murine contact hypersensitivity model: Toward the understanding of allergic contact dermatitis. J Invest Dermatol 2013, 133, 303-315; Grabbe, S.; Schwarz, T. Immunoregulatory mechanisms involved in elicitation of allergic contact hypersensitivity. Immunol Today 1998, 19, 37-44). Sequential immune cell migration that occurs in this phase is guided mainly by the secretion of chemoattractant factors secreted by keratinocytes and immune cell infiltrates. Keratinocytes, neutrophils, and mast cells play an important role in this early phase response to trigger the antigen-nonspecific inflammatory cascade. The haptens first come into contact with keratinocytes and release proinflammatory mediators like IL-1β and TNFα (Watanabe, H.; Gaide, O.; Petrilli, V.; Martinon, F.; Contassot, E.; Rogues, S.; Kummer, J. A.; Tschopp, J.; French, L. E. Activation of the il-1beta-processing inflammasome is involved in contact hypersensitivity. J Invest Dermatol 2007, 127, 1956-1963; Nielsen, M. M.; Lovato, P.; MacLeod, A. S.; Witherden, D. A.; Skov, L.; Dyring-Andersen, B.; Dabelsteen, S.; Woetmann, A.; Odum, N.; Havran, W. L., et al. Il-1beta-dependent activation of dendritic epidermal t cells in contact hypersensitivity. J Immunol 2014, 192, 2975-2983). The presence of these cytokines increases the vascular permeability and recruits both mast cells and T cells to the site of challenge (Dudeck, A.; Dudeck, J.; Scholten, J.; Petzold, A.; Surianarayanan, S.; Kohler, A.; Peschke, K.; Vohringer, D.; Waskow, C.; Krieg, T., et al. Mast cells are key promoters of contact allergy that mediate the adjuvant effects of haptens. Immunity 2011, 34, 973-984). Mast cell degranulation and histamine release leads to the release of chemokines CXCL1 and CXCL2, both factors are recruitment signals for neutrophils (Honda, T.; Matsuoka, T.; Ueta, M.; Kabashima, K.; Miyachi, Y.; Narumiya, S. Prostaglandin e(2)-ep(3) signaling suppresses skin inflammation in murine contact hypersensitivity. J Allergy Clin Immunol 2009, 124, 809-818 e802). Neutrophil recruiting chemokine factors are released within 2-3 hours of the challenge and neutrophils start invading the tissue around that time (Honda, T.; Matsuoka, T.; Ueta, M.; Kabashima, K.; Miyachi, Y.; Narumiya, S. Prostaglandin e(2)-ep(3) signaling suppresses skin inflammation in murine contact hypersensitivity. J Allergy Clin Immunol 2009, 124, 809-818 e802; Weber, F. C.; Nemeth, T.; Csepregi, J. Z.; Dudeck, A.; Roers, A.; Ozsvari, B.; Oswald, E.; Puskas, L. G.; Jakob, T.; Mocsai, A., et al. Neutrophils are required for both the sensitization and elicitation phase of contact hypersensitivity. J Exp Med 2015, 212, 15-22; Christensen, A. D.; Skov, S.; Haase, C. The role of neutrophils and g-csf in dnfb-induced contact hypersensitivity in mice. Immun Inflamm Dis 2014, 2, 21-34; Mitsui, G.; Mitsui, K.; Hirano, T. Kinetic profiles of sequential gene expressions for chemokines in mice with contact hypersensitivity. Immunol Lett 2003, 86, 191-197). Following the recruitment of neutrophils into the tissue, around 6-9 hours post challenge, T cell recruiting factors like CXCL9 and CXCL10 are released in the tissue. This allows memory T cells produced during the sensitization phase to invade the inflamed tissue (Mitsui, G.; Mitsui, K.; Hirano, T. Kinetic profiles of sequential gene expressions for chemokines in mice with contact hypersensitivity. Immunol Lett 2003, 86, 191-197). For Th1-type haptens the APCs in the skin interact with T cells to produce cytokines like IFNγ and IL-17 (Honda, T.; Egawa, G.; Grabbe, S.; Kabashima, K. Update of immune events in the murine contact hypersensitivity model: Toward the understanding of allergic contact dermatitis. J Invest Dermatol 2013, 133, 303-315). At this stage cytokines produced by T cells further activate skin resident cells and augment inflammation which peaks at around 24-48 hours post challenge in the animal model, which is represented by dermal edema and spongiosis. Once human beings are sensitized to an allergen it takes anywhere between a few hours to 2 days to show a phenotypic change on the skin like rash and blistering.

There has been some debate over the past 10 years over the role of cytotoxic T cell (CD8+) and helper T cells (Th, CD4+) in the CHS response (Vocanson, M.; Hennino, A.; Chavagnac, C.; Saint-Mezard, P.; Dubois, B.; Kaiserlian, D.; Nicolas, J. F. Contribution of cd4(+) and cd8(+) t-cells in contact hypersensitivity and allergic contact dermatitis. Expert review of clinical immunology 2005, 1, 75-86; Saint-Mezard, P.; Berard, F.; Dubois, B.; Kaiserlian, D.; Nicolas, J. F. The role of cd4+ and cd8+ t cells in contact hypersensitivity and allergic contact dermatitis. Eur J Dermatol 2004, 14, 131-138). Some studies have shown that CD8+ T cells are the main players whereas; CD4+ T cells exert a more regulatory function in this regard (CD4+ CD25+ regulatory T cells) (Kish, D. D.; Gorbachev, A. V.; Fairchild, R. L. Cd8+ t cells produce il-2, which is required for cd(4+)cd25+ t cell regulation of effector cd8+ t cell development for contact hypersensitivity responses. J Leukoc Biol 2005, 78, 725-735; Gorbachev, A. V.; Fairchild, R. L. Induction and regulation of t-cell priming for contact hypersensitivity. Crit Rev Immunol 2001, 21, 451-472; Gorbachev, A. V.; Heeger, P. S.; Fairchild, R. L. Cd4+ and cd8+ t cell priming for contact hypersensitivity occurs independently of cd40-cd154 interactions. J Immunol 2001, 166, 2323-2332). However, most animal studies using this model have demonstrated that haptens can polarize the development of T helper cells into specific sub-types (Th1, Th2 and Th17), which depends on the cytokine milieu in the skin (Peiser, M. Role of th17 cells in skin inflammation of allergic contact dermatitis. Clin Dev Immunol 2013, 2013, 261037; Peiser, M.; Tralau, T.; Heidler, J.; Api, A. M.; Arts, J. H.; Basketter, D. A.; English, J.; Diepgen, T. L.; Fuhlbrigge, R. C.; Gaspari, A. A., et al. Allergic contact dermatitis: Epidemiology, molecular mechanisms, in vitro methods and regulatory aspects. Current knowledge assembled at an international workshop at BfR, Germany. Cell Mol Life Sci 2012, 69, 763-781). 1-fluoro-2,4-dinitrobenzene (DNFB) and 2-deoxyurushiol are both Th1 haptens. In the following study, the role of nanoparticles (NPs) in the modulation of the CHS response is examined at a mechanistic level, with a focus on 20 nm SiNPs for the experiments along with the DNFB as the chemical allergen. The experiments conducted here are geared towards understanding whether the presence of SiNPs alter the bioavailability of DNFB in skin or modifies the cytokine milieu in the challenge phase, changing the cascade of immune events in the elicitation phase. DNFB-protein adducts in the skin, cytokines from the various treatment groups (multiplexed cytokine analysis) and the immune cells in the skin were quantified using both immunohistochemistry and flow cytometry.

The methods and materials are now described.

Step-Wise Application Experiments (Silica NP 20 nm/DNFB Dosing Sequence)

C57BL/6 hairless mice were used for all the experiments. Silica NPs (SiNP, 20 nm) were applied on the co-challenge ear either before or after DNFB application. Briefly, C57BL/6 hairless mice were sensitized to 0.05% DNFB (4:1 acetone:olive oil vehicle, volume ratio) on day 0 as previously described. 5 days after sensitization, the ear thickness was measured using digital calipers (Kroeplin #C11OT). On day 5, the right ear was challenged with 0.2% DNFB (4:1 acetone:olive oil vehicle, volume ratio). The left ear was either pre-treated with 20 nm SiNPs 3, 2, and 1 hours before DNFB application or post-treated 1, 2, and 3 hours after DNFB application. The ear swelling response was measured 24 hours after the challenge using digital calipers. In a different set-up, mice sensitized to 0.05% DNFB were challenged with glutathione-coated quantum dots (GSH-QDs) or 20 nm SiNPs (left ear alone). 1 hour after the NP application on the left ear, the NPs were wiped off using cotton-tipped applicators soaked in 1× phosphate buffered saline (PBS). Both the right and the left ear were then treated with 0.2% DNFB.

### Quantification of DNFB-Protein Adducts

Ear tissue was obtained from mice sensitized with 0.05% DNFB and challenged with 0.2% DNFB (right ear) and 0.2% DNFB+NP combination (left ear). The NPs used in this experiment included 20 nm and 400 nm SiNPs, glutathione coated QDs (GSH QDs), and multi-walled carbon nanotubes (MWCNTs). Tissue was also collected from treated mice 24 hours after the DNFB application. OxyBlot™ protein oxidation detection kit was obtained from Chemicon International (Catalog No: S7150) to quantify DNFB-protein adducts. Briefly, whole skin homogenates were separated by SDS PAGE and transferred to 0.2 micron nitrocellulose by Western blot. The blots were treated with the primary and secondary antibodies in the Oxyblot kit, and the protein band intensity was quantified by a Gel-Doc system (Biorad). The loading of each sample was controlled by the colorimetric analysis of a ponceau total protein stain.

### Multiplexed Cytokine Analysis

Mice were sensitized to 0.05% DNFB on day 0 and challenged with 0.2% DNFB (right ear), 0.2% DNFB+20 nm SiNPs (left ear) on day 5. The animals were divided into 4 groups, 1) no treatment controls 2) sacrificed 2 hours post-challenge, 3) sacrificed 12 hours post-challenge, and 4) sacrificed 24 hours post-challenge. 4 animals were included in each treatment group. Mice were sensitized with 0.05% DNFB and challenged with 0.2% DNFB (right ear) and vehicle alone (left ear) and were included to observe systemic effects (no NP treatment). These mice were sacrificed 2, 12, and 24 hours after challenge (N=4 per group). After the animals were euthanized, the right and left ears as well as the lymph nodes (axillary and brachial) were collected for analysis and stored at −80° C. until further processing. The tissue was thawed and homogenized in T-PER™ tissue protein extraction reagent (ThermoFisher Scientific, Cat No: 78510) to extract protein. The protein extracted in each sample was quantified using a Pierce™ BCA (bicinchoninic acid) Protein Assay Kit (ThermoFisher Scientific, Cat No: 23225). The protein concentrations were normalized to 5 μg/ml for each sample before the samples were utilized for Milliplex analysis. Customized Milliplex® Multiplex Analysis Kit for Luminex was purchased from EMD Millipore. The kit included a panel to analyze 16 mouse cytokines and chemokines: IL-4, IL-12, IL-6, IL-2, IL-10, TNFα, IFNγ, GM-CSF, TGFβ, IL-1α, IL-1β, KC, MIP-2, RANTES, IL-17, Il-5 and IL-3.

### Immunohistochemistry (IHC) and Cell Counts

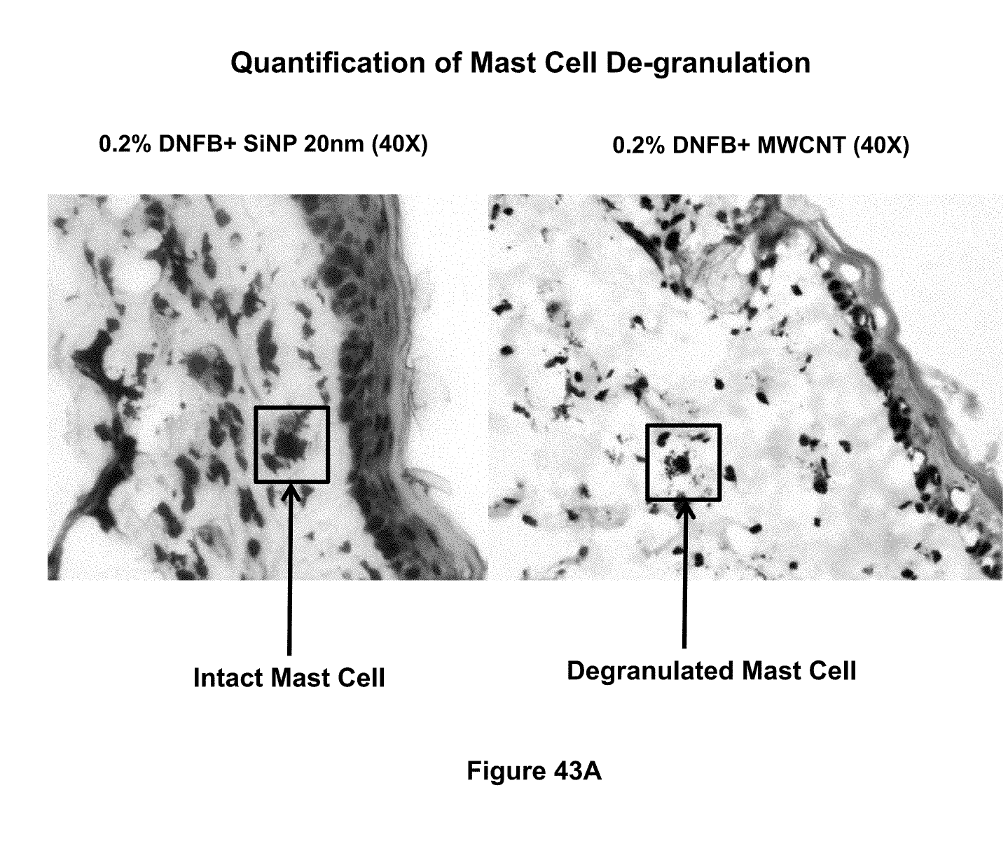

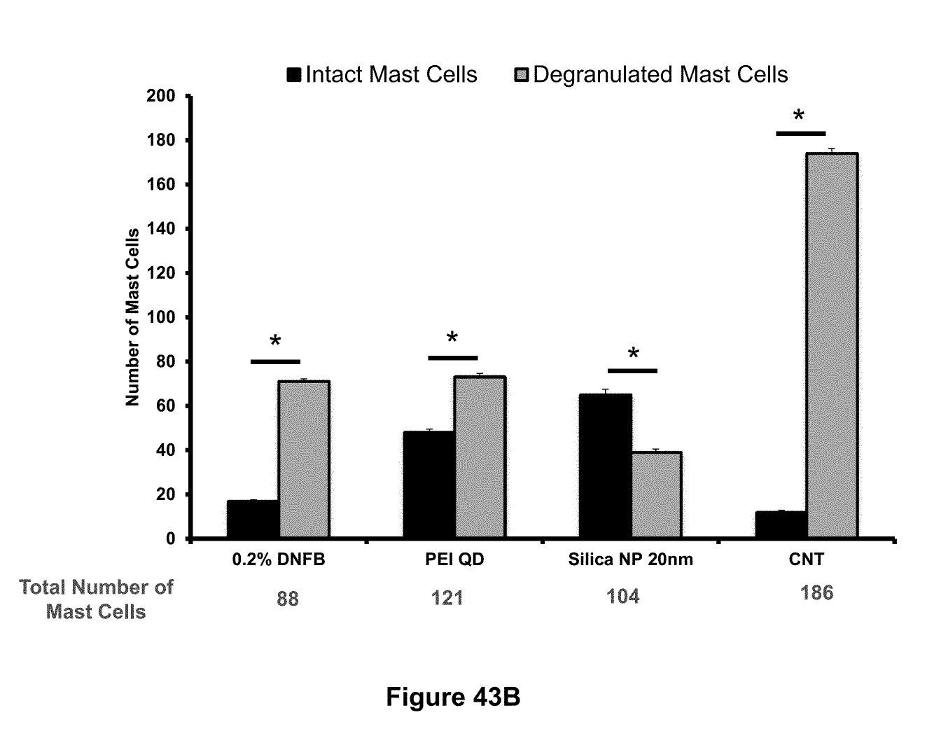

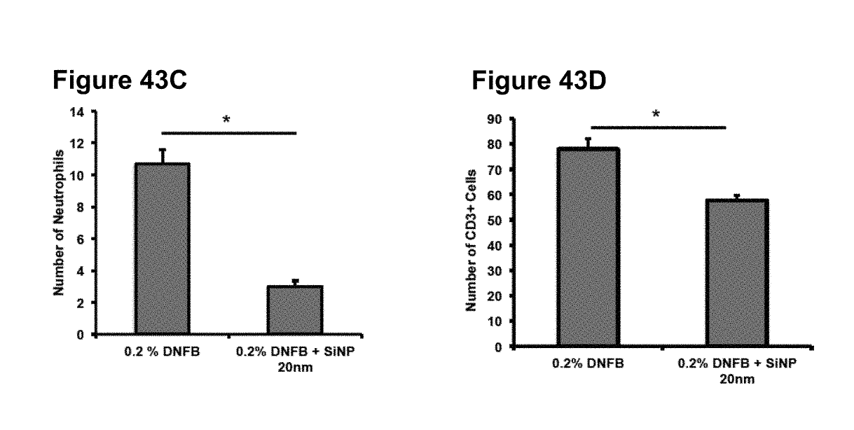

Ear tissue was obtained from mice sensitized with 0.05% DNFB and challenged with 0.2% DNFB (right ear) and 0.2% DNFB+20 nm SiNPs (left ear). In some analyses ear tissue co-challenged with 0.2%DNFB+PEI QDs and 0.2% DNFB+CNTs was included as a comparison (mast cell quantification). The tissue was frozen at −80° C. until further processing. O.C.T compound (Fischer Healthcare™ Tissue Plus™) was used as an embedding medium for frozen tissue to ensure optimal cutting temperature. The tissues were sectioned into 5 μm thick sections using Thermo Scientific Cryotome FE. Geimsa stain (Sigma Aldrich, Catalog No: GS) was used to stain the sections for mast cells. The stain colors the nuclei in varying shades of purple and the cytoplasm is stained blue to light pink. Eosinophils and red blood cells are stained shades of pink and bright orange, whereas mast cell granules are stained purple. The sections were imaged at 40× magnification on a Nikon Eclipse E800 microscope and RT3 camera/spot advanced software (version 4.6). With the help of an expert from surgical pathology, the number of mast cells (intact vs. de-granulated) were counted in each section (FIG. 43A). The sections were stained with hematoxylin and eosin stain (H&E) using standard procedures and the sections were imaged at 40× magnification (Nikon Eclipse E800). Each section was then used to locate cells with multi-lobed nuclei in order to quantify neutrophils in each treatment group. Similarly, sections were also stained for T cell infiltrates using the anti-CD3 antibody (eBioscience). Anti-PD-L1 antibody (eBioscience) was used to stain tissues and the sections were quantified using ImageJ software.

### Flow Cytometry Analysis

Mice were sensitized to 0.05% DNFB and challenged with 0.2% DNFB (right ear) and 0.2% DNFB+20 nm SiNPs (left ear). Mice were sacrificed at 2, 12, and 24 hours post-challenge (N=3-4) and ear tissue as well lymph nodes (axillary and brachial) were collected from the different treatment groups. The ears were split into two halves with forceps before digestion in 1 mg/mL collagenase in phosphate buffered saline at 37° C. for 30 minutes. 0.3M calcium chloride was used to activate the collagenase and 0.5M EDTA was added to each sample to quench the digestion. The lymph nodes were mashed using frosted slides before digestion in collagenase. The ears were mashed on a tea strainer using a plunger from a 10 ml syringe (BD Biosciences) after the digestion. The cell suspensions were collected in 15 mL tubes and centrifuged at 1600 rpm (4° C.). The supernatant was removed and the cell pellets were distributed in individual Eppendorf tubes for staining. The panel of fluorophore-conjugated antibodies used for staining included CD3, CD4, CD8, MHCII and Gr-1 (eBioscience). The data was collected using an 18-color LSRII flow cytometer and analyzed using FlowJo.

### Data and Statistical Analysis

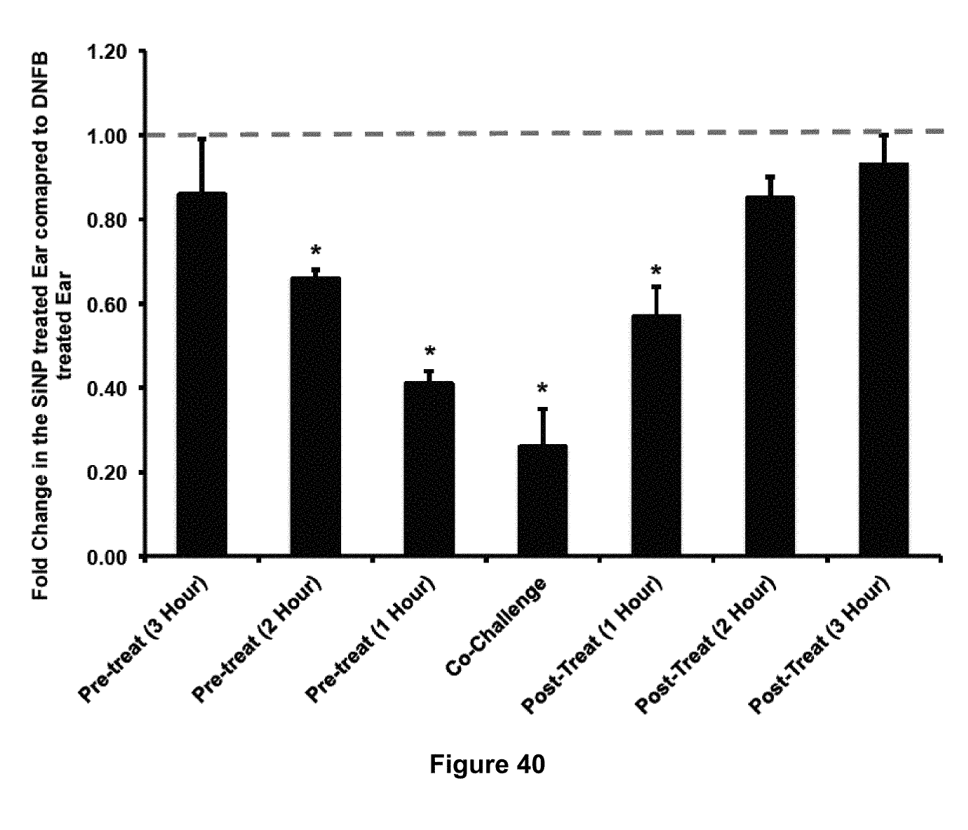

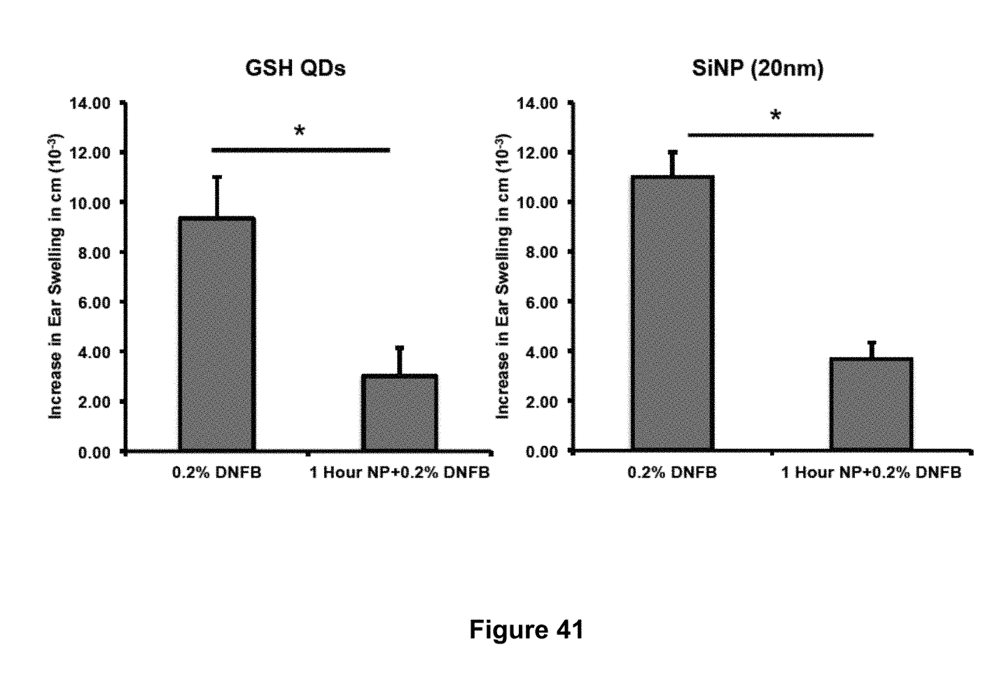

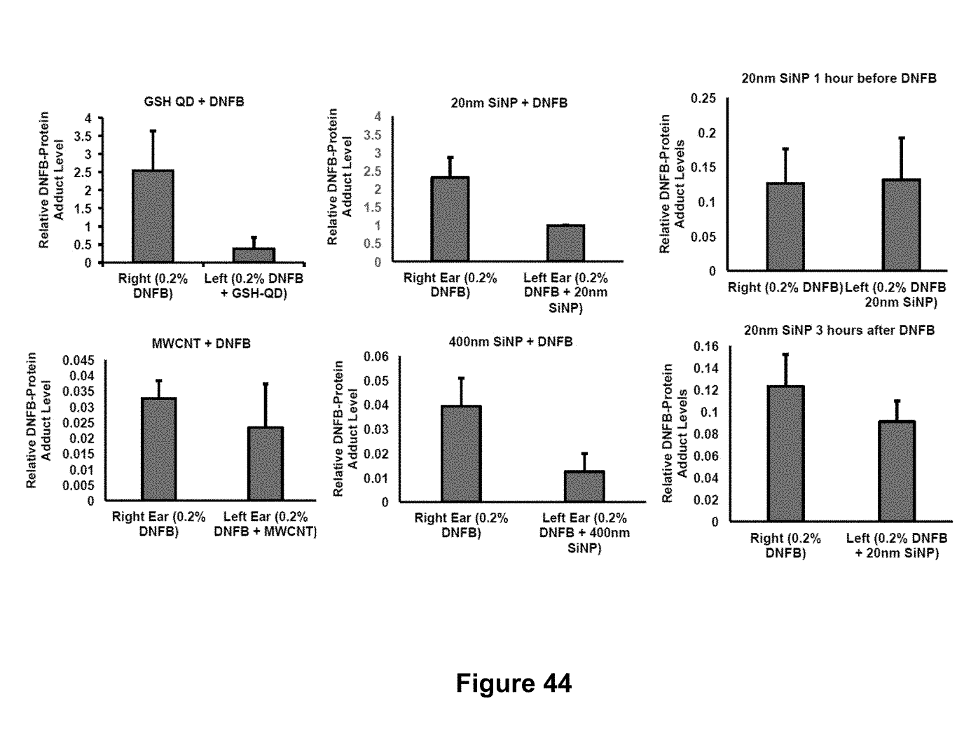

The number of animals used for each study varied between 3-5. Student's t-Test (paired, 2-tailed) was used to analyze differences between different treatments in the experiments represented in (FIG. 40, FIG. 41, FIG. 44, FIG. 43A through FIG. 43C) (p<0.05 was considered significant). 2-way ANOVA with post-hoc Tukey analysis was used to analyze the data for the multiplexed cytokine studies. The statistics and number of animals used for each study are described in detail under each figure legend.

The results and discussion are now described.

The co-challenge experiments demonstrated an astonishing ability of 20 nm silica NPs (SiNPs) to suppress the ear swelling response in the elicitation phase of the CHS response. The results from previous studies led to several important questions including bioavailability of DNFB to interact with skin proteins in the presence of SiNPs as well as altered DNFB-protein adduct formation. Mice were either pre-treated or post-treated with SiNPs in the challenge phase before or after the application of DNFB, respectively (left ear). The swelling response was measured using digital calipers with the right ear serving as control (DNFB alone treated). It was observed that when the SiNPs were applied 1 hour and 2 hour before DNFB application, the ear swelling response was inhibited (p<0.05) (FIG. 40). However, pre-treatment with SiNPs 3 hours before DNFB application had no effect. The immunosuppressive effect was observed when SiNPs were applied 1 hour after DNFB application (p<0.05) but post-treatment at 2 and 3 hours was ineffective (FIG. 40). This demonstrates that there is a small window of time after the skin is exposed to the allergen when the NPs are able to inhibit the response. The result is also important from the viewpoint of designing a topical preventative treatment because individuals will often get exposed to the hapten first and then apply the topical treatment to prevent the allergic reaction. Co-challenge experiments, although important from the perspective of screening NPs, do not present a real life scenario of allergen exposure. Key events like the influx of neutrophils as well as mast cell degranulation occurs between 1-6 hours after the challenge and it is possible application of NPs beyond the 2 hour window is unable to stop this initial cascade that eventually triggers a full blown response. The preliminary data obtained in the pre-treatment studies indicate that NP-based topical therapeutic when applied 1-2 hours before exposure, very similar to sunscreen use, can possibly prevent a full blown skin allergic response. In a separate study, GSH-QDs and 20 nm SiNPs applied on the mouse ears were wiped off 1 hour after application and the ears were then treated with 0.2% DNFB. A significant reduction in the ear swelling response was observed in both cases (p<0.05) (FIG. 41). This suggests that NPs are not merely blocking DNFB penetration through the stratum corneum since topical NPs were wiped off and the immunosuppressive effect is possibly the ability of NPs to either alter the antigen presentation response or alter the DNFB-protein adduct formation.

Previous studies have shown that DNFB-protein adduct formation, measured by western blotting with a DNP specific antibody, appear less in ears co-challenged with GSH-QDs+DNFB compared to ears treated with DNFB alone. However, quantification of DNFB-protein adducts was not significantly different when analyzed using densitometry (FIG. 37). After the preliminary screen, the DNFB-protein adducts were quantified in ear tissue treated with other NPs. As described earlier, DNFB is a low molecular weight chemical allergen that interacts with skin proteins to form a neoantigen that is recognized by the immune system as ‘altered self’ (Kaplan, D. H.; Igyarto, B. Z.; Gaspari, A. A. Early immune events in the induction of allergic contact dermatitis. Nat Rev Immunol 2012, 12, 114-124). DNFB-protein interaction creates a dinitrophenyl (DNP) moiety and the antibody against DNP in this assay detects DNP conjugated cellular proteins. Multi-walled CNT (MWCNT, carboxylated, 30 nm pore diameter, 5-20 μm length), 20 nm SiNPs and 400 nm SiNPs were analyzed and all groups appeared to show slight decreases in DNFB-protein adduct formation compared to ears treated with DNFB alone; however, there were no significant differences between the co-challenged ear and the DNFB alone treated ear in all three treatment groups (FIG. 44). Hence, with the detection of adducts in skin, a proinflammatory response would be expected. Next, the effect of NPs on adduct formation was examined when applied at different time-points pre and post DNFB application. The negative charged SiNPs (20 nm) were applied either 1 hour before DNFB or 3 hours after DNFB in the challenge phase. The analysis of DNFB-protein adducts showed no significant difference in DNFB-protein adducts between these ears and the DNFB alone treated samples (FIG. 44). However, the mice treated with 20 nm SiNP 1 hour before DNFB displayed reduced ear swelling despite the high DNFB-protein adduct levels detected (FIG. 40). This evidence demonstrates that the mechanism of action is more complex than simply reduced bioavailability of DNFB in the skin.

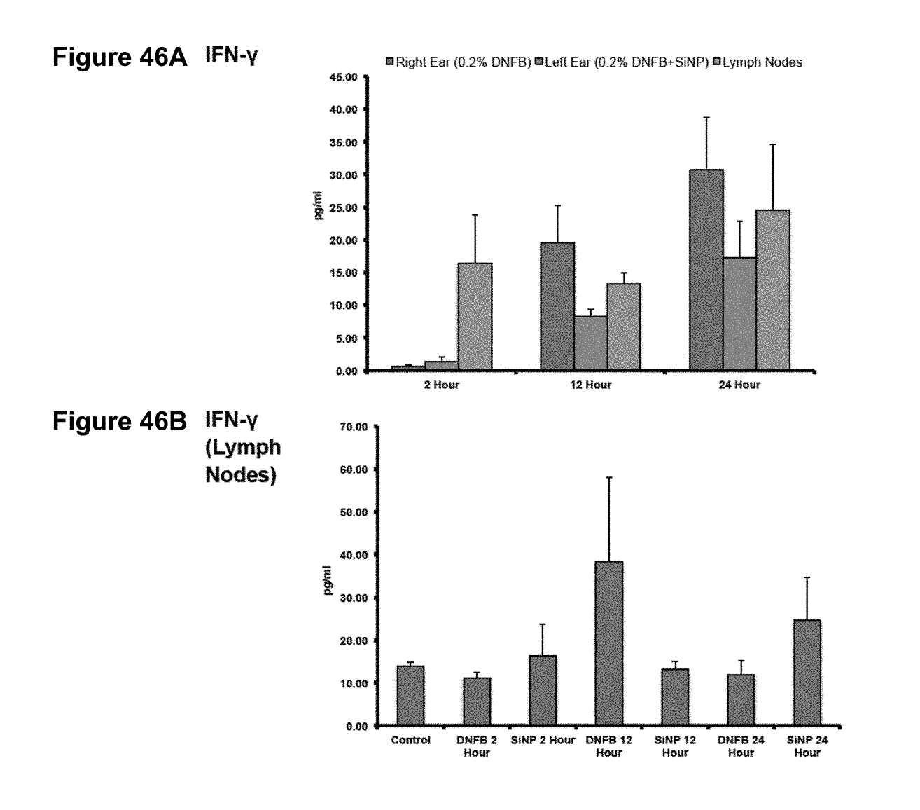

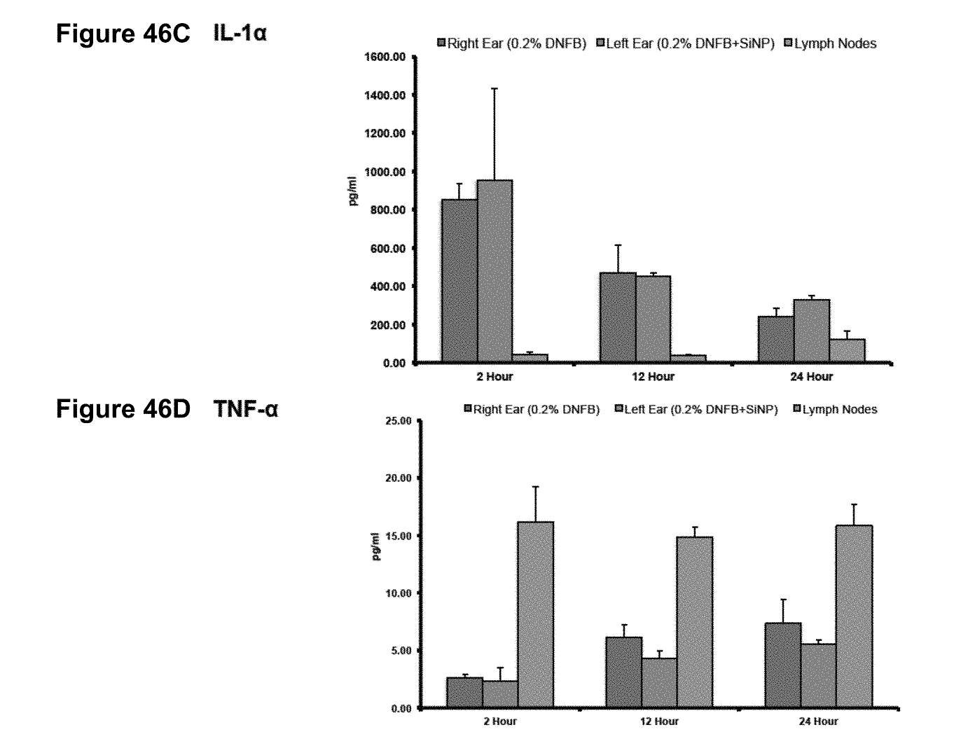

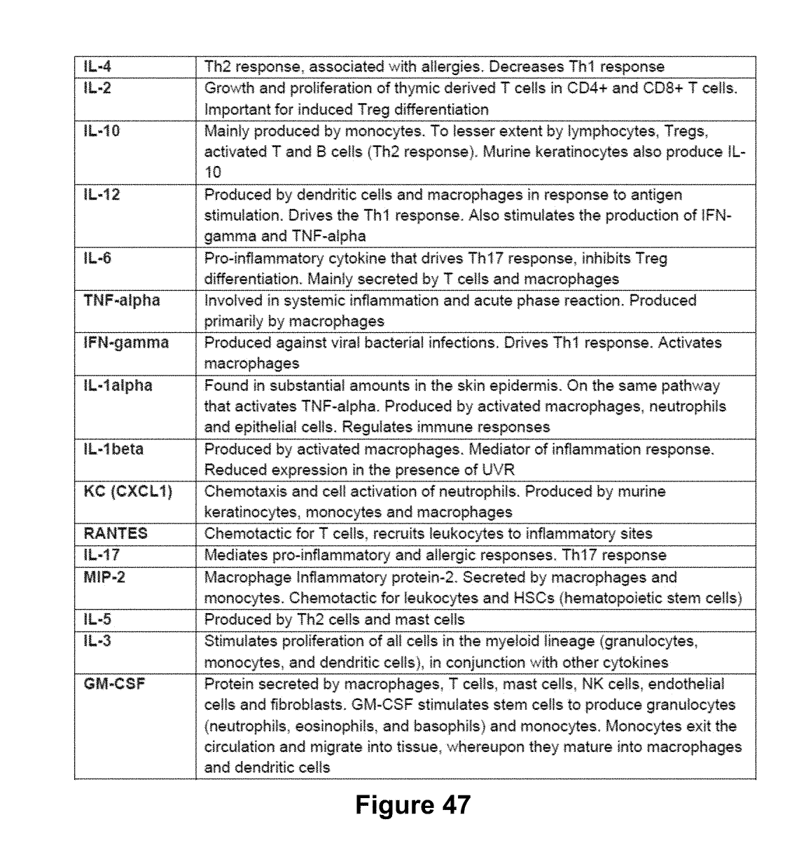

The cytokine and chemokine levels in the ear tissue were analyzed using the multiplexed Luminex assay. Ear tissue was obtained from mice sensitized with 0.05% DNFB and challenged with 0.2% DNFB (right ear) and 0.2% DNFB+20 nm SiNPs (left ear). Lymph nodes were also included in the analysis to observe systemic effects. The panel included 16 different cytokines and chemokines were included in the analysis and have been described in detail in (FIG. 47). Interleukin (IL) cytokines including IL-4, IL-12, IL-2, IL-17, IL-5, IL-3 and GM-CSF were below the level of detection (LOD) in most of the treatment groups as determined by the standards run for each 96-well plate. IL-10 did not exhibit any particular trends in the treatment groups and RANTES was detected only in the lymph nodes with no significant differences between the different treatment groups. IFNγ levels were higher in the 0.2% DNFB alone treated ear compared to the co-challenged ear (0.2% DNFB+20 nm SiNPs) at the 12 and 24 hour time-point, however these differences were not statistically significant (p=0.18 and p=0.15, respectively) (FIG. 46A, FIG. 46B). IL-1α levels were high in both the right and the left ear tissue with no significant differences between treatment groups (FIG. 46C). The baseline concentration of IL-1α measured in the control tissue was an average of 995.33 pg/ml, which was higher than any of the treatment groups. TNFα concentrations in the ear tissue were close to that measured in the controls around 3.7 pg/ml (FIG. 46D). The levels TNFα measured in the lymph nodes was higher compared to the ear tissue, however, there were no significant differences between the three treatment groups (2, 12, and 24 hour post-challenge).

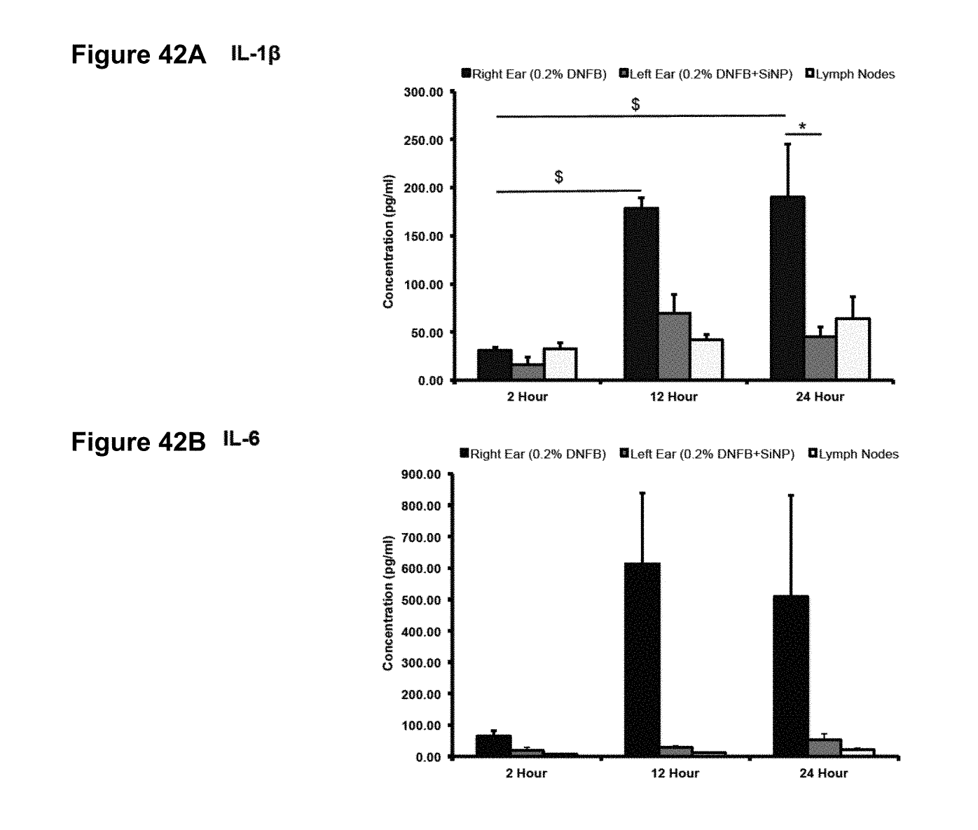

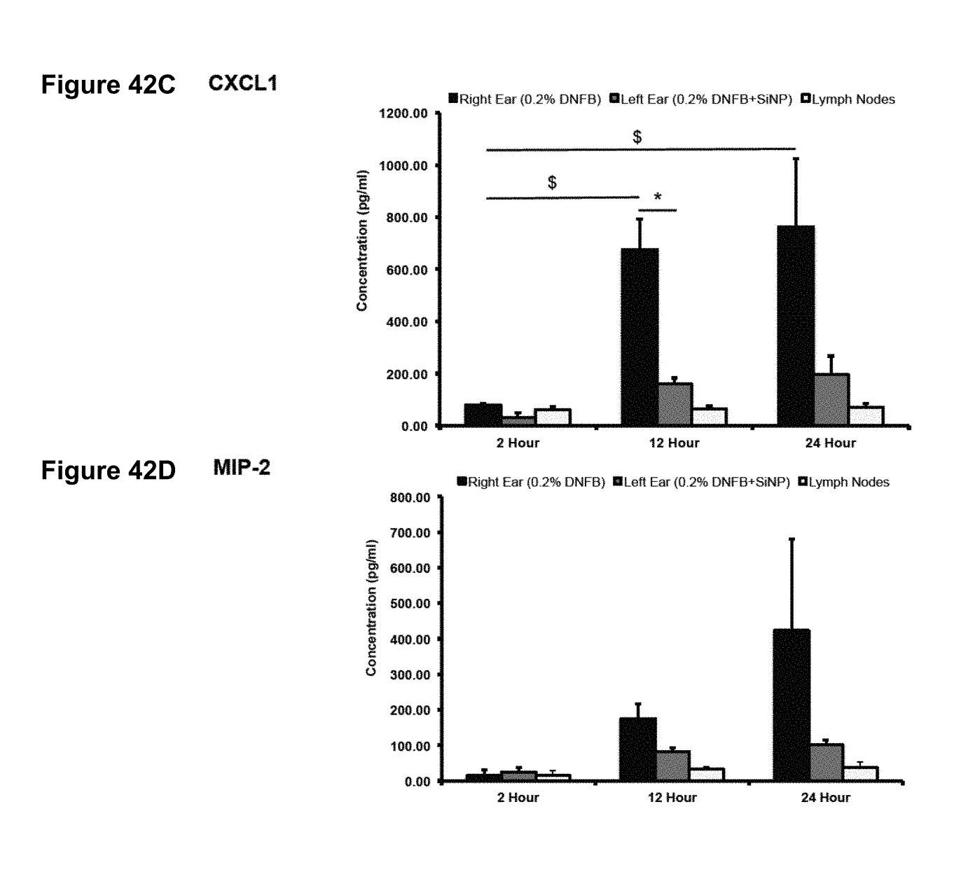

Some important trends were observed in the analysis of IL-1β, IL-6, KC (CXCL1), and MIP-2. Baseline concentration of IL-1β measured in the control tissue was around 8.7 pg/mL. The ear tissue treated with both DNFB+20 nm SiNPs had lower levels of IL-1β at the 12-hour time point compared to the DNFB alone treated tissue (FIG. 42A). At the 24-hour time point the co-challenged left ear had significantly lower levels of IL-1β compared to the DNFB alone treated ear (p<0.05) (FIG. 42B). A 6-fold increase of IL-1β levels was also observed at 24 hours in the DNFB alone treated ear tissue compared to the 2-hour time point (FIG. 42A). Similarly, IL-6 concentrations were higher in the DNFB alone treated ear at 12 and 24 hours compared to the co-challenged ear (DNFB+20 nm SiNP) (FIG. 42B). While the baseline concentration measured in the control ear was 4.68 pg/mL, the concentrations measured in the DNFB treated ear were 613.6 pg/ml (21 fold higher than SiNP treated ear) and 508.61 pg/ml (9 fold higher than SiNP treated ear) in the DNFB alone treated ear at 12 and 24 hours, respectively. The IL-6 concentrations measured in the lymph nodes of the various treatment groups were not significantly higher than the control tissue. KC (CXCL1) concentration was significantly higher in the DNFB alone treated ear tissue at the 12-hour time point compared to the DNFB+20 nm SiNP treated tissue (10 fold higher concentration) (p<0.05) (FIG. 42C). A similar trend was observed between the two ears at 24 hours as well (3 fold higher concentration), however, the difference was not statistically significant (FIG. 42C). The concentration of KC in the DNFB alone treated ear at the 12 and 24-hour time point was significantly higher than the 2-hour treatment group (p<0.05). Finally, the concentration of MIP-2 was also higher in the DNFB alone treated ears as compared to the DNFB+20 nm SiNP treated ears at the 12 and 24 hour time points, however, these differences were not statistically significant (FIG. 42D).

These observations are important in context of various events that occur in the elicitation/challenge phase of the CHS response. The diverse cytokine milieu secreted by the skin keratinocytes as well as the various immune cells that transverse through the skin during the hypersensitivity reaction leads to first an antigen non-specific and then an antigen specific activation (Grabbe, S.; Schwarz, T. Immunoregulatory mechanisms involved in elicitation of allergic contact hypersensitivity. Immunol Today 1998, 19, 37-44). Key cytokine signals secreted by keratinocytes, neutrophils, and mast cells are responsible for the first phase of the inflammatory response followed by the recruitment of antigen specific T cells to generate a full blown reaction (Honda, T.; Egawa, G.; Grabbe, S.; Kabashima, K. Update of immune events in the murine contact hypersensitivity model: Toward the understanding of allergic contact dermatitis. J Invest Dermatol 2013, 133, 303-315). It has been shown that the haptens trigger the keratinocytes to secrete pro-inflammatory cytokines like IL-1β, which increase expression of adhesion molecules (ICAM-1 and P/E-selectins) on endothelial cells, guiding the mast cells and T cells to the site of allergen application (ear tissue in this study) (Watanabe, H.; Gaide, O.; Petrilli, V.; Martinon, F.; Contassot, E.; Rogues, S.; Kummer, J. A.; Tschopp, J.; French, L. E. Activation of the il-1beta-processing inflammasome is involved in contact hypersensitivity. J Invest Dermatol 2007, 127, 1956-1963). Lower concentrations of IL-1β were observed in the cochallenged ear indicating that the presence of SiNPs in the challenge phase is mitigating the initial release of pro-inflammatory factors by the keratinocytes (FIG. 42A). Mast cells that invade the tissue in this early phase of the elicitation response degranulate and release histamine, further increasing vascular permeability and leading to an influx of neutrophils. Keratinocytes and mast cells release neutrophil recruiting chemokines such as CXCL1 and CXCL2 that initiate neutrophil recruitment to the tissue (Dudeck, A.; Dudeck, J.; Scholten, J.; Petzold, A.; Surianarayanan, S.; Kohler, A.; Peschke, K.; Vohringer, D.; Waskow, C.; Krieg, T., et al. Mast cells are key promoters of contact allergy that mediate the adjuvant effects of haptens. Immunity 2011, 34, 973-984). Chemokine analysis in the study revealed significantly lower concentrations of CXCL1 (KC) and CXCL2 (MIP-2) in the ear tissue co-challenged with SiNPs compared to the tissue treated with DNFB alone, again indicative of the fact that NP presence alters the chemokine milieu, possibly inhibiting the early influx (2-3 hours post-challenge) of key perpetuators like neutrophils and mast cells into the challenge site (ear tissue) (FIG. 42C, FIG. 42D). IL-6 is a pro-inflammatory cytokine and in the study the levels of this cytokine was diminished when the ear tissue was co-challenged with DNFB+20 nm SiNPs, indicating reduced inflammation at the challenge site when compared to the ear treated with DNFB alone (FIG. 42B). The altered cytokine milieu in the co-challenged tissue compared to the inflamed tissue challenged with DNFB alone suggested the possibility of impaired immune cell recruitment to the site of challenge in the early phase of the response, which was examined via immunohistochemistry, histology, and flow cytometry.

Ear tissue was obtained from mice sensitized with 0.05% DNFB and challenged with DNFB (right ear) and DNFB+NP combination (left ear). Mice were sacked 24 hours after challenge and the tissue was cryosectioned, and stained with Geimsa stain. The stain distinctly stains mast cell granules bright purple. The sections were imaged at 40× magnification and mast cells were manually quantified as described in (FIG. 43A). The mast cells were divided into two groups, intact and degranulated. The numbers of degranulated mast cells were observed to be significantly higher in the ear tissues treated with 1) DNFB alone, 2) co-challenged with DNFB+PEI QD, and 3) co-challenge with DNFB+CNT; all cases where significant ear swelling response was measured (FIG. 43A). In fact, in the case of CNTs where an enhanced ear swelling response was observed post co-challenge, an extremely high number of degranulated mast cells are present in the tissue. This also explains the presence of edema in tissue where swelling response is observed since mast cells burst leading to histamine release and edema. However, the number of intact mast cells was significantly higher that degranulated mast cells in ear tissue co-challenged with DNFB+20 nm SiNPs; a treatment group where ear swelling response is mitigated by the application of NPs (FIG. 43A).

Mast cells are key players in the early phase of the CHS response leading to histamine release (degranulation), vascular dilation and cytokine release leading to the recruitment of neutrophils and T cells to the inflamed tissue. The presence of fewer numbers of degranulated mast cells in tissue treated with SiNPs indicates that immune suppression modulated by the NPs occurs within 2-4 hours of the challenge. Treatment not only alters the cytokine milieu, but NP presence in the tissue prevents mast cell degranulation as well. Ear tissue was also stained with hemotoxylin and eosin (H&E) and anti-CD3 (T cell) to quantify neutrophils and T cells. Stained tissue was imaged at 40× magnification and the cell populations were quantified manually. Cells with multi-lobed nuclei in the H&E stain were counted as neutrophils and cells stained with anti-CD3 antibody were counted as T cells. It was observed that both neutrophil and T cell numbers were significantly lower in the ear tissue co-challenged with DNFB+20 nm SiNPs compared to tissue treated with DNFB alone (FIG. 43B, FIG. 43C). This tissue histology corresponds to the cytokine data where a significantly lower concentration of CXCL1, a neutrophil recruiting chemokine, was measured in the ear co-challenged with DNFB+20 nm SiNP.

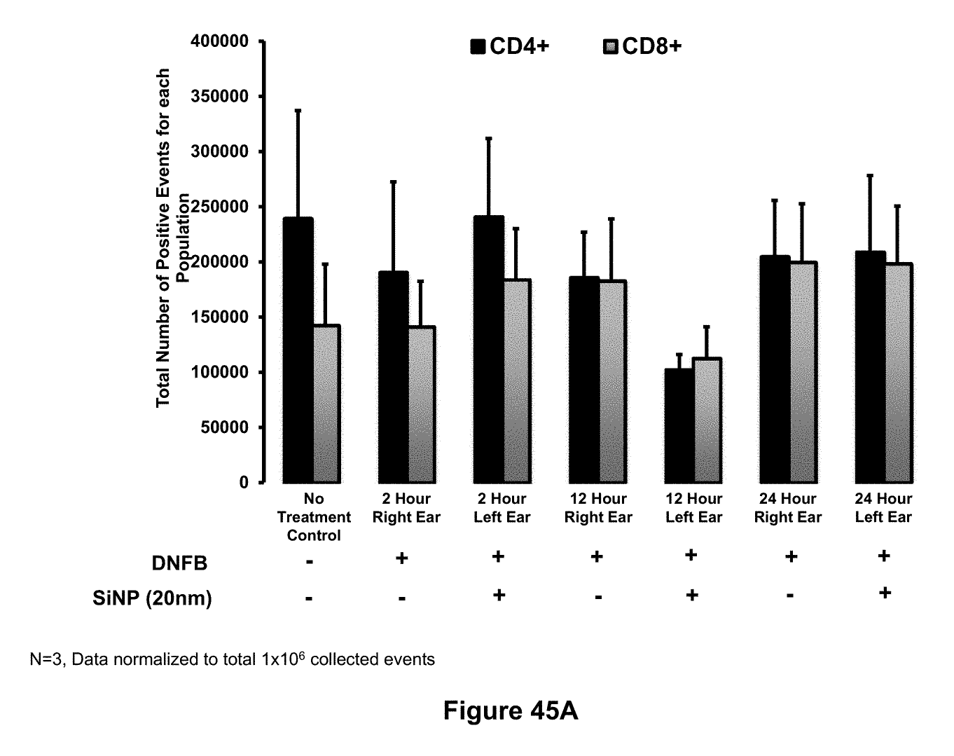

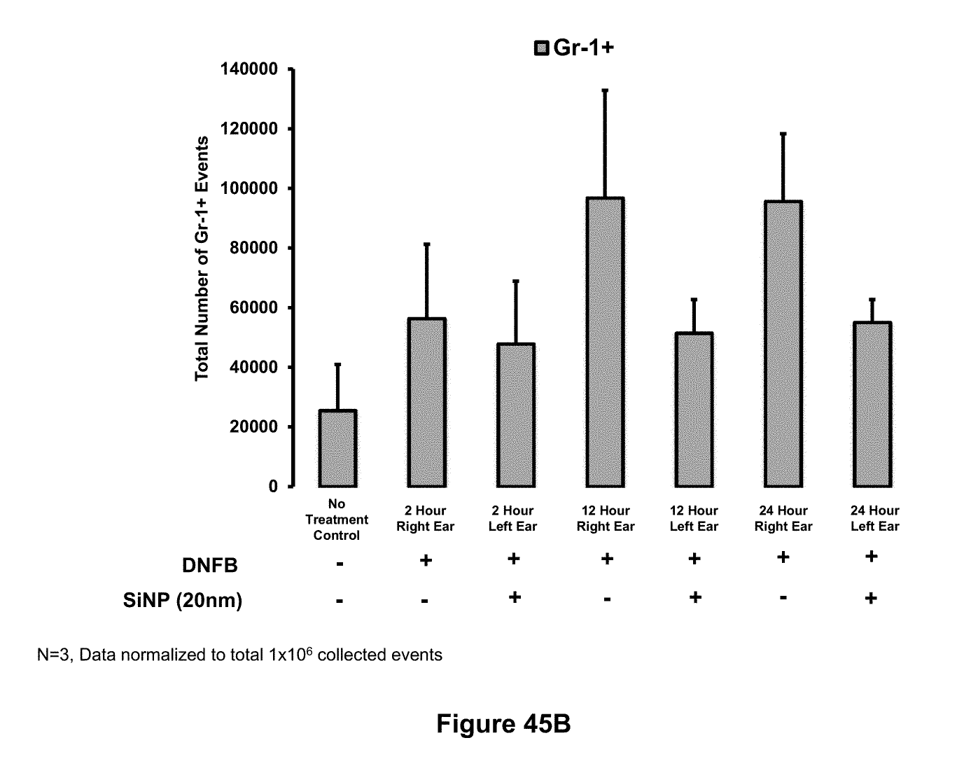

The cell populations present in the ear tissue as well as the lymph nodes were analyzed at 2, 12, and 24 hours post-challenge using flow cytometry. These time points paralleled those used in the cytokine study. No significant trends were observed in the lymph nodes and this may be due to the fact that T cell proliferation in the lymph nodes occurs primarily during the sensitization phase, which was not examined. Neutrophils (Gr-1+) exhibited a similar trend as observed in the IHC data. The total number of Gr-1+ events was lower in the co-challenged ear (DNFB+20 nm SiNP) compared to the DNFB alone treated ear at the 2, 12, and 24-hour time points; however these differences were not significant (N=3) (FIG. 45A, FIG. 45B). CD4+ and CD8+ T cell numbers were observed to be comparable to the controls in both the ear treatments at the 2 and 24-hour time point. The 12-hour treatment group showed trend toward a lower influx of T cell in the tissue co-challenged with 20 nm SiNP compared to DNFB treated alone; however, this difference was also not significant.

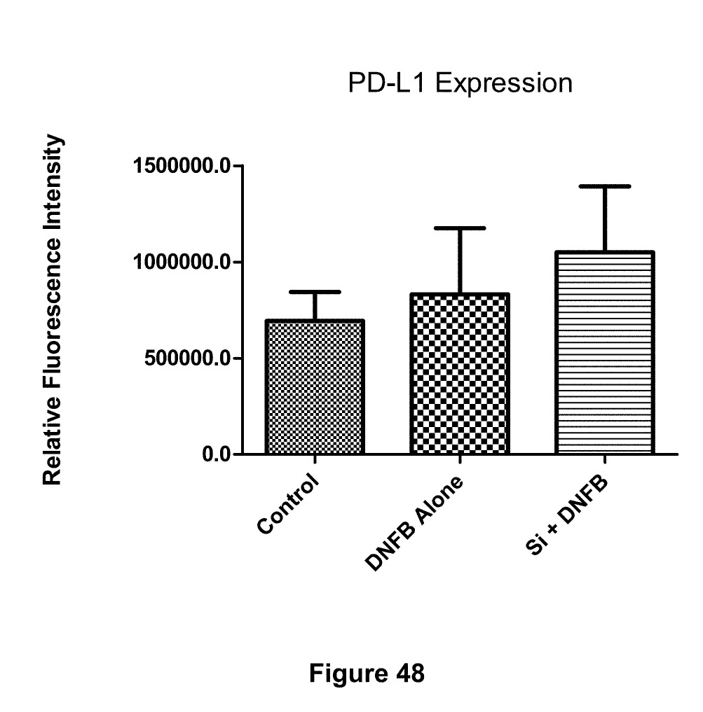

Next, it was examined whether the presence of NPs in the challenge phase alters T cell-dendritic cell interactions. Immune checkpoints that regulate T cell proliferation include cell surface receptors like CD28 and PD-1 (Alegre, M. L.; Frauwirth, K. A.; Thompson, C. B. T-cell regulation by cd28 and ctla-4. Nat Rev Immunol 2001, 1, 220-228). PD-1 interacts with PD-L1 (CD274, programmed death ligand-1) on antigen presenting cells to transmit an inhibitory signal that negatively impacts T cell proliferation (Ohaegbulam, K. C.; Assal, A.; Lazar-Molnar, E.; Yao, Y.; Zang, X. Human cancer immunotherapy with antibodies to the pd-1 and pd-l1 pathway. Trends Mol Med 2015, 21, 24-33). Hitzler et al., observed an upregulation in the PD-L1 expression in human skin biopsies from patients with ACD post-challenge with Nickel (Hitzler, M.; Majdic, O.; Heine, G.; Worm, M.; Ebert, G.; Luch, A.; Peiser, M. Human langerhans cells control th cells via programmed death-ligand 1 in response to bacterial stimuli and nickel-induced contact allergy. PLoS One 2012, 7, e46776). Ear tissues were stained using an anti-PD-L1 antibody in various treatment groups and intensity was quantified using ImageJ analysis. Results obtained using IHC showed no significant differences in PD-L1 expression in control, DNFB alone treated and SiNP (20 nm)+DNFB treated tissue (FIG. 48). This indicates that suppression of the immune response is potentially upstream of the T cell influx time-point in the cascade of events observed in the challenge phase.

The results indicate that the presence of SiNPs in the challenge phase alters the ear swelling response, which is possibly an early effect, within 1-2 hours of allergen application. The step-wise application studies indicate that if incorporated in a topical therapeutic, the inflammatory cascade could possibly be mitigated when applied within the 2-hour window of exposure. The cytokine and flow cytometry analysis also shows that the presence of NPs mitigates the release of early phase cytokines like IL-1β, KC, as well as MIP-2 and hinders the recruitment of neutrophils to the site of elicitation, a step that is essential to trigger the inflammatory cascade. The results obtained from IHC demonstrate that mast cell degranulation, which leads to the release of T cell recruiting cytokines is significantly lower in the tissue treated with 20 nm SiNP compared to the DNFB alone treated group. Preliminary data obtained using flow cytometry analysis shows a decrease in neutrophil influx at the 12 and 24-hour time point post-challenge, and a decrease in T cell influx at the 12 hour time point can be observed in the DNFB+20 nm SiNP treated tissue.

Studies conducted here show an astonishing ability of NPs (silica, gold, and silver NPs) to suppress skin allergy in the contact hypersensitivity (CHS) mouse model in the elicitation phase of the response. This is important from the perspective of designing a therapeutic because most individuals are already sensitized to the allergen. The intrinsic immunosuppressive properties of NPs can be exploited for treating skin inflammatory disorders including allergic contact dermatitis (poison ivy, nickel skin allergy). Using a mouse model of contact hypersensitivity employing 1-fluoro-2,4-dinitrobenzene (DNFB) as the chemical sensitizer (Th1 hapten), it was observed that some NPs, depending upon core composition, surface coating, and charge, can suppress the ear swelling response whereas other NPs, such as CNTs and TiO₂, exacerbate symptoms. It is an important discover that NPs have an intrinsic ability to modulate skin immune responses. Experiments conducted in these studies demonstrate that the immunosuppressive effect is not due to the lack of bioavailability of the sensitizer (DNFB), but rather the alteration of the cytokine milieu and inhibition of the influx of immune cells into the NP treated tissue. NPs also are shown to inhibit the allergic response when applied 1-2 hours before the allergen (sensitizer, DNFB) or 1 hour after the application of the sensitizer, demonstrating that the immunosuppressive effect is present only when NPs are applied within a small window of time. Data examined herein illustrate that the immunosuppressive effect occurs in the elicitation phase and alters some key immune events like neutrophil influx and mast cell degranulation.

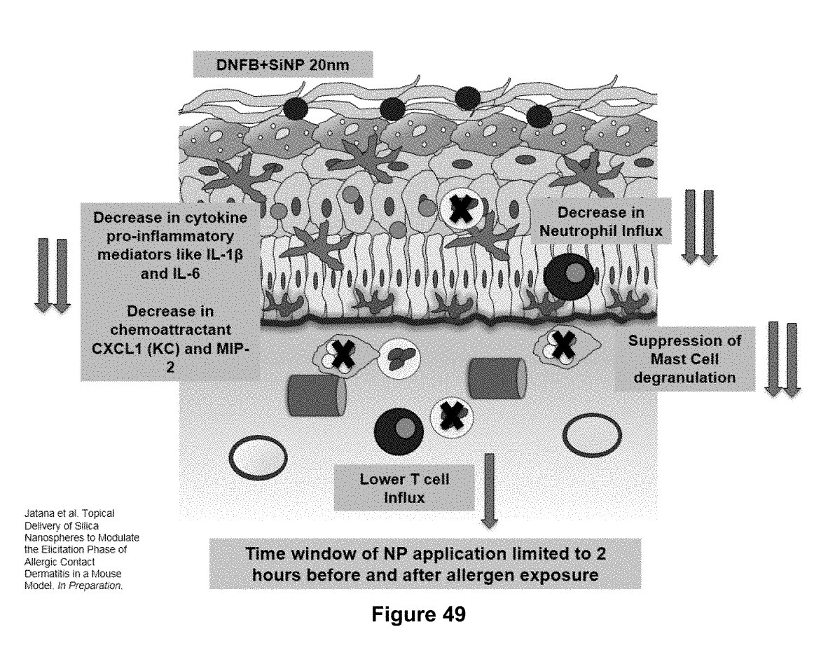

Macrophages, skin keratinocytes, and Langerhans cells mainly produce IL-1β after epicutaneous hapten application (Nambu, A.; Nakae, S. Il-1 and allergy. Allergol Int 2010, 59, 125-135). The presence of this cytokine downregulates the expression of E-cadherin, an adhesion molecule, that allows Langerhans cell migration from the skin (Jakob, T.; Udey, M. C. Regulation of e-cadherin-mediated adhesion in Langerhans cell-like dendritic cells by inflammatory mediators that mobilize langerhans cells in vivo. J Immunol 1998, 160, 4067-4073). IL-6 is another pro-inflammatory cytokine produced by macrophages and T cells, which negatively impacts regulatory T cell (Tregs) differentiation. In the models presented herein, the presence of Silica NPs (20 nm) along with DNFB in the challenge phase decreased the production of pro-inflammatory mediators such as IL-1β and IL-6 (FIG. 49). The levels of both IL-1β and IL-6 were lower at the 12 and 24-hour time point after challenge in the NP co-challenged tissue compared to tissue treated with the allergen alone. The levels of CXCL1 or KC (a neutrophil recruiting chemokine) and MIP-2 secreted by macrophages (a leukocyte recruiting chemokine) were also decreased in NP co-challenged tissue in comparison to allergen alone treated group (FIG. 49). Subsequently, when the number of cellular infiltrates was quantified using flow cytometry and immunohistochemistry, a lower number of neutrophils and T cells was observed in the NP co-challenged tissue. The presence of NPs in the challenge phase was demonstrated to impact the secretion of key cytokine/chemokine mediators that ultimately recruit immune cells to the local tissue to mediate the inflammatory response. NP presence in the challenge phase significantly decreased mast cell degranulation in the NP co-challenged tissue compared to allergen treated group.

Mast cell degranulation can occur via three primary mechanisms. First, the pathogen/allergen can directly interact with Toll-like receptors (TLR) to release cytokine mediators like IL-6, GM-CSF and TNF (Marshall, J. S. Mast-cell responses to pathogens. Nat Rev Immunol 2004, 4, 787-799). Mast cell activation via TLRs does not cause degranulation, which has been positively correlated with ear swelling. Some allergens (example: nickel) can induce the production of reactive oxygen species (ROS), which leads to the generation of low molecular weight hyaluronic acid (HA) (Kaplan, D. H.; Igyarto, B. Z.; Gaspari, A. A. Early immune events in the induction of allergic contact dermatitis. Nat Rev Immunol 2012, 12, 114-124). Second, IgE crosslinking on cell surface Fc receptors leads to degranulation and release of histamines, proteases as well as cytokine mediators (Marshall, J. S. Mast-cell responses to pathogens. Nat Rev Immunol 2004, 4, 787-799). An example of this is allergic asthma, which is a Type I hypersensitivity response. T cells produce Th2-cytokines in response to some allergens that lead to the production of IgE by B cells, which leads to mast cell degranulation (Pernis, A. B.; Rothman, P. B. Jak-stat signaling in asthma. J Clin Invest 2002, 109, 1279-1283). In the CHS model presented herein, DNFB is a Th1 hapten that drives the delayed type IV hypersensitivity response mediated by T cells, demonstrating that degranulation by IgE crosslinking is not the primary mechanism. The third possible mechanism is via the complement-receptor mediated activation, particularly C5a (C5aR), which also causes mast cell degranulation (Marshall, J. S. Mast-cell responses to pathogens. Nat Rev Immunol 2004, 4, 787-799).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating skin inflammation in a subject in need thereof, the method comprising:
administering a therapeutically effective amount of a composition comprising at least one nanoparticle (NP) to a site of skin inflammation of the subject.

2. The method of claim 1, wherein the at least one NP is selected from the group consisting of: silica nanosphere, porous silicon nanoshard, quantum dot, gold nanoparticle, and silver nanoparticle.

3. The method of claim 2, wherein the at least one NP is the quantum dot comprising a neutrally charged coating, or a negatively charged coating, or a glutathione coating.

4. The method of claim 2, wherein the at least one NP is a quantum dot that is lipophilic, organic, or a cadmium-selenide/zinc sulfide (CdSe/ZnS) quantum dot capped with octadecyl amine ligands (ODA).

5. The method of claim 2, wherein the silica nanosphere has a diameter of about 10 nm to about 1200 nm.

6. (canceled)

7. The method of claim 2, wherein the porous silicon nanoshard has a porosity between about 20% and about 80%.

8. The method of claim 2, wherein the porous silicon nanoshard has a diameter of about 1 nm to about 1000 nm.

9. (canceled)

10. The method of claim 1, wherein the composition is administered topically.

11. The method of claim 1, wherein the skin inflammation is associated with at least one selected from the group consisting of: chemical irritation, contact dermatitis, and an autoimmune disorder.

12. The method of claim 1, wherein the skin inflammation is associated with at least one selected from the group consisting of allergic contact dermatitis (ACD), irritant contact dermatitis, atopic dermatitis (AD), photoallergic dermatitis, and contact hypersensitivity (CHS).

13. The method of claim 1, wherein the skin inflammation comprises at least one of: swelling, redness, barrier dysfunction, pruritus (itch), and induration (tissue hardening).

14. A composition for the treatment of skin inflammation, the composition comprising an effective amount of at least one nanoparticle (NP), wherein the at least one NP suppresses an immune response in skin.

15. The composition of claim 14, wherein the at least one NP is selected from the group consisting of: silica nanosphere, porous silicon nanoshard, quantum dot, gold nanoparticle, and silver nanoparticle.

16. The composition of claim 15, wherein the at least one NP is a quantum dot comprising a neutrally charged coating, a negatively charged coating, or a glutathione coating.

17. The composition of claim 15, wherein the at least one NP is a quantum dot that is lipophilic, organic, or a cadmium-selenide/zinc sulfide (CdSe/ZnS) quantum dot capped with octadecyl amine ligands (ODA).

18. The composition of claim 15, wherein the silica nanosphere has a diameter of about 10 nm to about 1200 nm.

19. (canceled)

20. The composition of claim 15, wherein the porous silicon nanoshard has a porosity between about 20% and about 80%.

21. The composition of claim 15, wherein the porous silicon nanoshard has a diameter of about 1 nm to about 1000 nm.

22. (canceled)

23. The composition of claim 15, wherein the at least one NP further comprises a polymer within the NP.
